(19) United States
(12) Patent Application Publication
(10) Pub. No.: US 2018/0284125 A1
(43) Pub. Date: Oct. 4, 2018

(54) PROTEOMIC ANALYSIS WITH NUCLEIC ACID IDENTIFIERS

(71) Applicants: THE BROAD INSTITUTE, INC., Cambridge, MA (US); MASSACHUSETTS INSTITUTE OF TECHNOLOGY, Cambridge, MA (US)

(72) Inventors: David Benjamin Gordon, Somerville, MA (US); Robert Nicol, Cambridge, MA (US); Christopher Voigt, Cambridge, MA (US); Yongjin Park, Cambridge, MA (US); Graham N. Rockwell, Cambridge, MA (US); Ritika Dwivedi, Cambridge, MA (US)

(21) Appl. No.: 15/557,447

(22) PCT Filed: Mar. 11, 2016
(86) PCT No.: PCT/US2016/022211

§ 371 Date: Sep. 11, 2017

Related U.S. Application Data
(60) Provisional application No. 62/131,805, filed on Mar. 11, 2015.

(51) Int. Cl. G01N 33/68; C12Q 1/6874; C12N 15/10

(52) U.S. Cl. CPC G01N 33/6818; C12N 15/1086; C12Q 1/6874

(57) ABSTRACT

Disclosed are methods and compositions for labeling target molecules or associated target molecule tags with origin-specific nucleic acid barcodes. The identity, quantity, and/or activity of target molecules originating from particular discrete volumes, such as droplets, for example water-in-oil emulsions, can be determined by determining the sequence of the origin-specific nucleic acid barcodes (optionally in combination with additional barcodes, such as one or more additional nucleic acid and/or peptide barcode).

### TECHNICAL FIELD

The present disclosure relates to methods and compositions for the determination of protein expression using nucleic acid identification sequences. Specifically this disclosure allows for the multiplex determination of protein expression from complex mixtures or cells and/or acellular systems.

### BACKGROUND

Biology can build intricate materials and chemical structures with atomic precision. Making complex structures using synthetic chemistry is difficult or cost prohibitive due to confounding factors such as low yields, the generation of toxic waste, and the complexity of synthetic schema. Genetic engineering has provided biological routes to the production of some chemicals (for example, butanediol, isobutanol, and farnescene). However, these examples do not reflect the potential complexity achievable with synthetic biology due to the relative simplicity of the structures of these chemicals. A central challenge in synthetic biology is the requirement for coordinated spatial and dynamic regulation of systems including many genes and pathways. Design paradigms to build such systems do not exist. Harnessing the potential of synthetic biology to generate complex molecules requires the development of tools including, for example, multi-part DNA assemblies and methods for characterizing gene expression products from such assemblies in massively multiplex form. This present disclosure meets some of those needs.

### SUMMARY

The disclosure provides methods and compositions for labeling target molecules or associated target molecule tags with origin-specific nucleic acid barcodes. The identity, quantity, and/or activity of target molecules originating from particular discrete volumes, such as droplets, for example water-in-oil emulsions, can be determined by determining the sequence of the origin-specific nucleic acid barcodes (optionally in combination with additional barcodes, such as one or more additional nucleic acid and/or peptide barcode). In specific examples, the methods and compositions of the disclosure can be used, for example, in the generation and characterization of complex synthetic genetic systems, which in turn can be used in applications of synthetic biology. Disclosed is a method of proteomic analysis that uses nucleic acid identifiers to maintaining information about the origin of the target molecules, such as target polypeptides, for example target proteins. In certain embodiments, the disclosed method includes: providing a sample comprising cells, or an acellular system, comprising one or more target polypeptides of interest and/or nucleic acids encoding target polypeptides of interest, optionally linking a peptide identifier elements that allows for multiplex readout of the presence and/or amount in the individual polypeptides of interest; segregating cells, single cells or a portion of the acellular system from the sample into individual discrete volumes; labeling the target polypeptides in the discrete volumes with origin-specific barcodes present in the discrete volumes to create origin-labeled target polypeptides, wherein the origin-labeled target polypeptides from each individual discrete volume comprise the same unique indexing nucleic acid identification or matched sequence; and detecting the nucleotide sequence of the origin-specific barcodes, thereby assigning the set of target polypeptides to a specific discrete volume, while maintaining information about sample origin of the target polypeptides; and/or labeling the target polypeptides in the discrete volume with distinguishable mass tags present in the discrete volumes to create mass tag labeled target polypeptides, wherein the mass tag labeled target polypeptides from each individual discrete volume comprise the distinguishable mass tag and/or mass tags matched to the peptide identifier element; and detecting the mass tags, thereby characterizing the set of target polypeptide in a specific discrete volume, while maintaining information about sample origin of the target polypeptide. Embodiments of the method further include assigning the set of target molecules to target nucleic acids (such as RNA, for examples mRNA, and/or DNA, such as cDNA) in the sample or set of samples while maintaining information about sample origin of the target molecules and target nucleic acids. Such methods include: labeling the target nucleic acids in the discrete volumes with the origin-specific barcodes present in the discrete volumes to create origin-labeled target nucleic acids, wherein the origin-labeled target nucleic acids from each discrete volume include the same, or matched, unique indexing nucleic acid identification sequence as the origin-labeled target molecules; and detecting the nucleotide sequence of the origin-specific barcodes, thereby assigning the set of target molecules to target nucleic acids in the sample or set of samples while maintaining information about sample origin of the target molecules and the target nucleic acids.

In some embodiments of the method, the one or more target molecules are linked to a molecule identifier element, such as a polypeptide identifier element, for example a polypeptide covalently linked to the polypeptide identifier element, optionally by a peptide linker. In specific examples, the molecule identifier elements within a single discrete volume include distinct molecule identifier elements, differentiated from the molecule identifier elements in other discrete volumes. In certain embodiments, the polypeptide identifier element includes an affinity tag and/or a peptide barcode, both of which allow for the separation, isolation, and/or identification of the molecules, for example target polypeptides, nucleic acid barcodes and/or target nucleic acids to which they are attached or otherwise coupled.

Embodiments of the method include labeling the target molecule by attaching origin-specific nucleic acid barcode to the target molecule, via a target molecule specific; binding agent and/or an specific binding agent that binds to an affinity tag linked to the target molecule, such as an antibody that includes or is otherwise coupled to an origin-specific nucleic acid barcode.

In some embodiments, liquid chromatography-mass spectrometry (LC-MS) is used to isolate, and/or determine the relative abundance of target molecules, either directly or via an affinity tag, which in some examples includes different affinity tags that have predictable mass difference.

Also disclosed is a barcode labeling complex that includes a solid or semi-solid substrate (such as a bead, for example a hydrogel bead) and a plurality of barcoding elements reversibly coupled thereto, wherein each of the barcoding elements includes an indexing nucleic acid identification sequence and one or more of a nucleic acid capture sequence that specifically binds to target nucleic acids and a specific binding agent that specifically binds to the target molecules.

The foregoing and other objects and features of the disclosure will become more apparent from the following detailed description, which proceeds with reference to the accompanying figures.

### BRIEF DESCRIPTION OF THE DRAWINGS

FIGS. 1A and 1B is a diagram showing protein an exemplary labeling scheme via encoded protein tags (or “polypeptide identifier elements” encoded with respective target polypeptide molecules). FIG. 1A shows a multi-gene construct and, for one of the genes, sequences 3′ to the coding sequence (CDS) and encoding a proteolytic cleavage site (TEV) and a peptide barcode are highlighted. FIG. 1B shows exemplary fusion proteins that can be encoded by a construct such as that illustrated in FIG. 1A. The fusion proteins include target proteins (P1-P96) with C-terminal encoded polypeptide identifier elements that each include a variable length peptide barcode and an affinity tag (T), such as FLAG, MYC, HA, etc. In the examples shown, the affinity tags are bound by antibodies containing isotope labels or cleavable nucleic acid barcodes, which allows for sample multiplexing on a massive scale.

FIG. 2 is a diagram showing an exemplary scheme for high-throughput proteomics and transcriptomics in emulsion droplets. Individual cells are encapsulated in droplets with individual beads containing cleavable RNA/DNA capture oligos and antibody/protein capture molecules, all with the same nucleic acid barcode, such as origin-specific barcodes. The barcodes attached to a single bead all have the same sequence, or matched sequences. Cells are allowed to express proteins and then can be lysed. In this example, RT buffer is added and cDNA is made from expressed mRNA. Barcodes released from the bead bind to the cDNA and proteins, resulting in cDNA and proteins that are labeled with the same, or matched, origin-specific barcodes.

FIG. 3 is a diagram showing three exemplary variants of the proteomics and DGE method, including (A) a method for co-labeling target proteins and encoding cDNAs for combined proteomics and transcriptomics, (B) a method utilizing antibodies specific for encoded affinity tags, and (C) a method utilizing antibodies specific for encoded affinity tags and variable length peptide barcodes.

FIG. 4 is a diagram showing an exemplary scheme for high-throughput proteomics via sequence determination, in which each distinct target protein (P1-P10) is fused to an encoded, protease-cleavable affinity tag (for example, AU1, T7, etc.). Within a discrete volume (for example, a well or droplet), the affinity tags are cleaved from the target proteins and are labeled with affinity tag-specific nucleic acid barcodes (via, for example, antibodies), which are then labeled with origin-specific nucleic acid barcodes (each optionally including a UMI), which bind to the affinity tag-specific barcodes. The cleaved, labeled affinity tags are purified (for example, by protein G affinity) and pooled from different discrete volumes. The sequence is determined for quantification of the nucleic acid barcodes of the pool, which each include affinity tag-specific portions and origin-specific portions.

FIG. 5 is a diagram showing an exemplary alternate scheme for high-throughput proteomics via nucleic acid sequence determination, in which each distinct target protein (P1-P5) is fused to an encoded polypeptide identifier element including both an affinity tag (FLAG) and a variable length peptide barcode. FIG. 5A illustrates a multi-gene construct, with the 3′ end of one gene highlighted as including a coding sequence (CDS) and encoding a cleavage site (TEV) and a peptide barcode. FIG. 5B shows that, within a discrete volume (for example, a well or droplet), a fusion protein including a target protein (P1-P5), linked to a polypeptide identifier element that includes both an affinity tag (FLAG) and a variable length peptide barcode, is labeled with a origin-specific nucleic acid barcode, for example via a C-terminal cysteine. Polypeptide identifier elements are cleaved from target proteins, enriched (for example, by anti-FLAG antibodies), pooled, and then fractionated (for example, by HPLC). Fraction-specific nucleic acid barcodes are then linked to origin-specific barcodes. The nucleic acid barcodes, including fraction-specific portions and origin-specific portions, can then be sequenced for quantification of protein levels, as shown in FIG. 5C.

FIG. 6 is a diagram showing a further scheme for high-throughput proteomics via sequencing, which combines the use of multiple affinity tags, as explained in connection with FIG. 4, and multiple variable length peptide barcodes, as explained in reference to FIG. 5. A C-terminal cysteine is used for the addition of origin-specific barcodes. Polypeptide identifier elements are cleaved, pooled, and enrichment for each affinity tag is carried out; followed by fractionation (for example, by HPLC). Fraction-specific nucleic acid barcodes are then added to the origin-specific and affinity tag-specific nucleic acid barcodes, and then can be cleaved and sequenced for quantification of protein levels.

FIG. 7 is a diagram showing rapid prototyping in cell-free expression systems. A cell free prototyping system (CFPS) mix (for example, a cell free extract from, for example, high value organism) is introduced into droplets of an emulsion that each include a single bead including barcode indices and a construct from a genetic library. RNA is expressed in the droplets and labeled with the barcodes, and analysis of digital transcript levels can be used for part (for example, promoters, terminators, etc.) quantification.

FIGS. 8A and 8B are schematics and Western blots showing the optimization of TEV digestion protocol enables high efficiency TEV digestion on MBP-TEV-GFP system. FIG. 8A, schematic representation of the MBP-GFP fusion protein bridged with the TEV recognition site. This complex can be detected by Anti-Maltose Binding Protein antibody by western blot. FIG. 8B, Western blot result for two biological replicates after TEV digestion on MBP-TEV-GFP complex. Without TEV digestion, the MBP-TEV-GFP fusion protein remains intact under identical treatment condition. TEV protease cleaves GFP from the fusion protein and only MBP can be detected.

FIG. 9 is a schematic representation of newly designed peptide tags, illustrated in the context of a genetic program. Each peptide sequence contains a TEV cleavage site followed by 8 amino acid peptide barcodes and 1X FLAG tag. This FLAG tag can be used for enriching barcode population with high yield and high specificity.

FIG. 10 is a schematic showing the construction of barcoded transcription units and characterization of chemically synthesized peptides. Transcription units with individually distinguishable peptide barcode are constructed with variation in RBS strength. Peptide that is corresponding to each of the barcode is also chemically synthesized and further characterized via HPLC and MALDI-TOF.

FIG. 11 shows the impact of inducing amber suppressor tRNA system. As induction of the suppressor system is increased, a decrease in nitrogenase activity is observed. Further optimization of inducer concentration to achieve maximal cell viability and amber suppression will be required.

FIG. 12A, expression of peptide barcodes in vivo with four different types of suppressor plasmids. Inducer concentrations match with height of the triangles.

FIG. 12B, gel shift assay result after oligo-anti FLAG antibody conjugation.

FIG. 12C, sensitivity of immune-PCR enables detection of signal with <100 fold less amount of antibody.

FIGS. 13A and 13B are schematic diagrams for two debugging plasmids. FIG. 13A, amber stop codon after the NifF CDS is mutated to Tyr. This positive control plasmid can be utilized for optimizing IP efficiency and TEV cleavage steps. FIG. 13B, arabinose inducible SupD expression allows Ser to be incorporated in four TAG stop codons in front of sfGFP. Only if the SupD is expressed and functional, the sfGFP can be synthesized.

FIG. 14 is a schematic representation of peptide inducible peptide barcoding systems. Inducible expression of Suppressor tRNA expression adds another layer of controllability to the system. Cells are lysed, reduced and cleaved with highly specific TEV protease, purified with FLAG antibody and analyzed by mass spectrometry.

FIGS. 15A and 15B show the optimization and evaluation of inducible translational suppression. FIG. 15A, SupD tRNA brings Serine to the amber stop codon on mRNA (TAG). System with 1X and 4X stop codon in front of the sfGFP is devised to test inducibility based on SupD tRNA system. FIG. 15B, suppression of amber stop codon from supD tRNA resulted ˜20-fold induction of GFP expression in sfGFP with 1X amber stop codon.

FIG. 16 is a schematic representation of the architecture of a transcription unit. Features for the architecture include unidirectional cistrons to increase transferability between clusters, improved transcriptional insulation between transcription units with double terminators, ribozyme insulator to keep downstream gene expression free from upstream genetic context. Small RNA (sRNA) barcode for improved tunability of the system and epitope tagged barcode for rapid prototyping of genetic constructs.

FIG. 17 is a schematic providing an overview for detecting inducible peptide polypeptide identifiers, in accordance with certain example embodiments.

FIG. 18 is a schematic showing a set of plasmid constructs encoding amber suppressor tRNAs (SupF and SupD) (a) a schematics of an example inducible polypeptide identifier system construct comprising one to four amber stop codons relative an N-terminus of sfGFP, and (c) a graph showing levels of inducible expression using the example inducible polypeptide identifier construct

FIG. 19 is a graph showing the results a growth assay demonstrating minor toxicity of the example inducible polypeptide identifier system.

FIG. 20 shows an example monocistronic inducible polypeptide identifier element construct (a & b), In this system, each fluorescent protein is represented with a specific variable peptide sequence having a distinguishable mass and further contain a FLG epitope to allow for enrichment of polypeptide identifier labeled populations only. Graphs (c) and (d) show inducible levels of expression.

FIG. 21 shows a schematic for isolating polypeptide identifiers (a) from an inducible construct comprising MBP and GFP with a cleavable TEV linker in between (c). Optimal TEV protease conditions were determined (b) and TEV concentration dependent increase in protease activity demonstrated (d).

FIG. 22 is a schematic representing the architecture of a proximity ligation based detection system, in accordance with certain example embodiments.

FIG. 23 is a schematic representing another architecture of a proximity ligation based detection system, in accordance with certain example embodiments.

FIG. 24 is a graph showing the results of a qPCR analysis of successful PLA extension.

FIG. 25 is a picture of a gel demonstrating production of high yield PLA probes conjugated to antibodies.

FIG. 26 is a graph showing the results of a qPCR test of PLA quantitation of GFP-HIS.

FIG. 27 is a graph showing the results of a qPCR test of PLA quantitation of GFP-HIS.

FIG. 28 is a schematic showing architecture of a self-reporting origin-specific nucleic acid identifier system, in accordance with certain example embodiments.

FIG. 29 is a schematic showing the architecture of an alternative proximity ligation detection system, in accordance with certain example embodiments.

FIG. 30 is a schematic showing the architecture of an alternative proximity ligation detection system, in accordance with certain example embodiments.

FIG. 31 is a schematic showing the architecture of an alternative proximity ligation detection system, in accordance with certain example embodiments.

### DETAILED DESCRIPTION

### I. Terms

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

As used herein, the singular forms “a,” “an,” and “the,” refer to both the singular as well as plural, unless the context clearly indicates otherwise. For example, the term “an origin-specific barcode” includes single or plural probes and can be considered equivalent to the phrase “an origin-specific barcode.”

As used herein, the term “comprises” means “includes.” Thus, “an origin-specific barcode” means “including an origin-specific barcode” without excluding other elements.

Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the disclosure, the following explanations of terms are provided:

Amplification: To increase the number of copies of a nucleic acid molecule, such as a nucleic acid molecule that includes an indexable nucleic acid identifier, such an origin-specific barcode as described herein. The resulting amplification products are typically called “amplicons.” Amplification of a nucleic acid molecule (such as a DNA or RNA molecule) refers to use of a technique that increases the number of copies of a nucleic acid molecule (including fragments). In some examples, an amplicon is a nucleic acid from a cell, or acellular system, such as mRNA or DNA that has been amplified.

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Antibody: A polypeptide ligand comprising at least a light chain and/or heavy chain immunoglobulin variable region (or fragment thereof) which specifically recognizes and binds an epitope of an antigen, such as a protein, or a fragment thereof. Antibodies can include a heavy and a light chain, each of which has a variable region, termed the variable heavy (VH) region and the variable light (VL) region. The term also includes recombinant forms such as chimeric antibodies (for example, humanized murine antibodies), heteroconjugate antibodies (such as, bispecific antibodies). An antibody or fragment thereof may be multispecific, for example, bispecific. Antibodies include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, an immunoconjugate, or a protein scaffold with antibody-like properties, such as fibronectin or ankyrin repeats. The antibody can have any of the following isotypes: IgG (for example, IgG1, IgG2, IgG3, and IgG4), IgM, IgA (for example, IgA1, IgA2, and IgAsec), IgD, or IgE.

In most mammals, including humans, whole antibodies have at least two heavy (H) chains and two light (L) chains connected by disulfide bonds. Each heavy chain includes a heavy chain variable region (V_{H}) and a heavy chain constant region (C_{H}). However, single chain V_{HH} variants, such as found in camelids, and fragments thereof, are also included. The heavy chain constant region includes three domains, C_{H}1, C_{H}2, and C_{H}3 and a hinge region between C_{H}1 and C_{H}2. Each light chain includes a light chain variable region (V_{L}) and a light chain constant region. The light chain constant region includes the domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

Included are intact immunoglobulins and the variants and portions of them well known in the art, such as Fab fragments, Fab′ fragments, F(ab)′₂ fragments, single chain Fv proteins (“scFv”), and disulfide stabilized Fv proteins (“dsFv”) Fd, Feb, or SMIP. An antibody fragment may be, for example, a diabody, triabody, affibody, nanobody, aptamer, domain antibody, linear antibody, single-chain antibody, or multispecific antibodies formed from antibody fragments. Examples of antibody fragments include: (i) a Fab fragment: a monovalent fragment consisting of V_{L}, V_{H}, C_{L}, and C_{H}1 domains; (ii) a F(ab′)₂ fragment: a bivalent fragment including two Fab fragments linked by a disulfide bridge at the hinge region; (iii) a Fd fragment: a fragment consisting of V_{H} and C_{H}1 domains; (iv) a Fv fragment: a fragment consisting of the V_{L} and V_{H} domains of a single arm of an antibody; (v) a dAb fragment: a fragment including V_{H} and V_{L} domains; (vi) a dAb fragment: a fragment consisting of a V_{H} domain or a V_{HH} domain (such a Nanobody™); (vii) a dAb fragment: a fragment consisting of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, for example, by a synthetic linker that enables them to be made as a single protein chain in which the V_{L} and V_{H} regions pair to form monovalent molecules (known as single chain Fv (scFv)). Antibody fragments may be obtained using conventional techniques known to those of skill in the art, and may, in some instances, be used in the same manner as intact antibodies. Antigen-binding fragments may be produced by recombinant DNA techniques or by enzymatic or chemical cleavage of intact immunoglobulins. An antibody fragment may further include any of the antibody fragments described above with the addition of additional C-terminal amino acids, N-terminal amino acids, or amino acids separating individual fragments.

An antibody may be referred to as chimeric if it includes one or more variable regions or constant regions derived from a first species and one or more variable regions or constant regions derived from a second species. Chimeric antibodies may be constructed, for example, by genetic engineering. A chimeric antibody may include immunoglobulin gene segments belonging to different species (for example, from a mouse and a human).

A human antibody refers to a specific binding agent having variable regions in which both the framework and CDR regions are derived from human immunoglobulin sequences. Furthermore, if the antibody contains a constant region, the constant region also is derived from a human immunoglobulin sequence. A human antibody may include amino acid residues not identified in a human immunoglobulin sequence, such as one or more sequence variations, for example, mutations. A variation or additional amino acid may be introduced, for example, by human manipulation. A human antibody of the present disclosure is not chimeric.

Antibodies may be humanized, meaning that an antibody that includes one or more complementarity determining regions (for example, at least one CDR) substantially derived from a non-human immunoglobulin or antibody is manipulated to include at least one immunoglobulin domain having a variable region that includes a variable framework region substantially derived from a human immunoglobulin or antibody.

Biotin-16-UTP: A biologically active analog of uridine-5′-triphosphate that is readily incorporated into RNA during an in vitro transcription reaction by RNA polymerases such as T7, T3, or SP6 RNA Polymerases. In some examples, biotin-16-UTP is incorporated into an origin-specific barcode (or any other barcode) during reverse transcription from a probe DNA template, for example during in vitro transcription with an RNA Polymerase, such as T7, T3, or SP6 RNA Polymerase.

Capture moieties: Molecules or other substances that when attached to another molecule, such as a nucleic acid barcode disclosed herein, allow for the capture of the targeting probe through interactions of the capture moiety and something that the capture moiety binds to, such as a particular surface and/or molecule, such as a specific binding molecule that is capable of specifically binding to the capture moiety, which in some embodiments is a target molecule, affinity tag and/or nucleic acid or peptide barcode. In specific examples, a capture moiety is biotin and a capture moiety specific binding agent is avidin or streptavidin.

Capture molecule or capture element: A molecule or complex capable of binding a target molecule (or a target molecule tag), for example, for isolating the target molecule (or target molecule tag) from a plurality or pool of target molecules (or target molecule tags). The target molecule (or target molecule tag) bound by the capture molecule may be labeled with a nucleic acid barcode and/or a target molecule tag. In certain embodiments, a capture molecule recognizes an epitope on the target molecule. In various embodiments, a capture molecule recognizes an epitope on the target molecule tag. A capture molecule can be attached to a substrate, such as a column, bead, hydrogel, filter, surface on a slide, matrix of a polymer gel, or an interior wall of a discrete volume. For example, one or more capture molecules may be bound to a column, such that a solution containing a mixture of target molecules (or target molecule tags) can be run through the column to isolate one or more particular target molecules (or target molecule tags) recognized by the capture molecules. Multiple target molecules (or target molecule tags) attached to a substrate may include identical target molecules (or target molecule tags) or may include more than one distinct target molecule (or target molecule tag). Non-limiting examples of types of capture molecules include the following: peptides, polypeptides, proteins, antibodies, antibody fragments, amino acids, nucleic acids, nucleotides, carbohydrates, polysaccharides, lipids, small molecules, organic molecules, inorganic molecules, and complexes thereof. In certain embodiments, a capture molecule is an antibody or antibody fragment, or an antigen or antigen fragment including an epitope.

Chimeric Polypeptide: The term “chimeric polypeptide” refers to a polypeptide that includes a combination of peptide sequences that are found in two or more different proteins or fragments of proteins, for example a target protein, and a polypeptide identifier element. The peptide sequences included in a chimeric polypeptide can be from naturally-occurring proteins or non-naturally-occurring proteins, and can be from the same organism or different organisms. However, the combination of sequences in a chimeric polypeptide is typically a sequence that is not found in nature.

Cleavage peptide: A peptide generated by proteolytic cleavage of a protein or polypeptide with a protein cleavage agent. Such proteolytic peptides include peptides produced by treatment of a protein with one or more endoproteases such as trypsin, chymotrypsin, endoprotease ArgC, endoprotease aspN, endoprotease gluC, and endoprotease lysC, as well as peptides produced by chemical agents such as cyanogen bromide, formic acid, and thiotrifluoroacetic acid.

Contacting: Placement in direct physical association, including both in solid or liquid form, for example contacting a sample with a nucleic acid barcode.

Conditions sufficient to detect: Any environment that permits the detection of the desired activity, for example, that permits detection and/or quantification of a nucleic acid, such as a nucleic acid barcode, a transcription product, and/or amplification product thereof.

Control: A reference standard. A control can be a known value or range of values indicative of basal levels or amounts or present in a tissue or a cell or populations thereof (such as a normal non-cancerous cell). A control can also be a cellular or tissue control, for example a tissue from a non-diseased state and/or exposed to different environmental conditions. A difference between a test sample and a control can be an increase or conversely a decrease. The difference can be a qualitative difference or a quantitative difference, for example a statistically significant difference.

Covalent modification reagent: A covalent modification reagent is a reactive molecule that can react with a functional group on another molecule, for example, one or more functional groups (such as —OH, —NH₂, —SH, —CO—, —COOH groups) found on amino acids, peptides, polypeptides and proteins. Covalent modification reagents can be used to prepare polypeptides that are isotopic analogs of one another or sets thereof. In one embodiment, sets of polypeptides are separately treated with two versions of a covalent modification reagent: one version that is isotopically-labeled and one version that is not. For example, one of peptides is reacted with a first covalent modification reagent and another set is treated with a second covalent modification reagent that is an isotopic analog of the first reagent. In other words, after reaction of the two sets with different reagents that have the same structure but different masses, the two sets of modified peptides will have the same structure but different masses and are thus isotopic analogs of each other. An example of this type of labeling scheme is the Isotope-coded Affinity Tag (ICAT) scheme, wherein two versions of reagents that react with specific functional groups on peptides are used to separately treat standard and sample peptides (either separated or part of the chimeric polypeptide or sample proteins, respectively). Examples of ICAT reagents include those described by Gygi et al. (Gygi et al., “Quantitative Analysis of Complex Protein Mixtures Using Isotope-coded Affinity Tags,” Nat. Biotechnol., 17: 994-999, 1999). In the method of Gygi et al., biotinylated iodoacetamide derivatives in a heavy form (deuterated) and in a light form are used to label cysteines of two protein extracts before treatment with a protein cleavage agent. The primary amine group is also a target for introducing an isotopic label, and may be used where peptides of interest do not contain cysteine. For example, the deuterated and non-deuterated forms of N-acetoxysuccinimide or acetate can be used to differentially label the N-terminus and the s-amino groups of lysines (see, for example, Ji et al., “Strategy for Qualitative and Quantitative Analysis in Proteomics Based on Signature Peptides, J. Chromatogr. B Biomed. Sci. Appl., 745: 187-210, 2000). Cleavable ICAT reagents are commercially available from Applied Biosystems (Foster City, Calif.).

Covalently linked: Refers to a covalent linkage between atoms by the formation of a covalent bond characterized by the sharing of pairs of electrons between atoms. In one example, a covalent link is a bond between an oxygen and a phosphorous, such as phosphodiester bonds in the backbone of a nucleic acid strand. In another example, a covalent link is one between nucleic acid barcode and a solid or semisolid substrate, such a bead, for example a hydrogel bead.

Chromatography: The process of separating a mixture, for example based on the properties of the molecules in the mixture, such as size, length, charge, hydrophobicity and the like. Typically, chromatography involves passing a mixture through a stationary phase, which separates molecules of interest from other molecules in the mixture and allows it to be isolated. Examples of methods of chromatographic separation include capillary-action chromatography such as paper chromatography, thin layer chromatography (TLC), column chromatography, fast protein liquid chromatography (FPLC), nano-reversed phase liquid chromatography, ion exchange chromatography, gel chromatography such as gel filtration chromatography, size exclusion chromatography, affinity chromatography, high performance liquid chromatography (HPLC), and reverse phase high performance liquid chromatography (RP-HPLC) amongst others.

Detect: To determine if an agent (such as a signal or particular nucleic acid, such a nucleic acid barcode, protein barcode, affinity tag, or protein) is present or absent. In some examples, this can further include quantification in a sample, or a fraction of a sample, such as a particular cell or cells or acellular system.

Detectable label: A compound or composition that is conjugated directly or indirectly to another molecule to facilitate detection of that molecule. Specific, non-limiting examples of labels include fluorescent tags, enzymatic linkages, and radioactive isotopes. In some examples, a label is attached to an antibody or nucleic acid to facilitate detection of the molecule antibody or nucleic acid specifically binds.

DNA sequencing: The process of determining the nucleotide order of a given DNA molecule. Generally, the sequencing can be performed using automated Sanger sequencing (AB13730xl genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®). In some embodiments, the identity of a nucleic acid is determined by DNA or RNA sequencing. Generally, the sequencing can be performed using automated Sanger sequencing (AB13730xl genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing.

In some embodiments, DNA sequencing is performed using a chain termination method developed by Frederick Sanger, and thus termed “Sanger based sequencing” or “SBS.” This technique uses sequence-specific termination of a DNA synthesis reaction using modified nucleotide substrates. Extension is initiated at a specific site on the template DNA by using a short oligonucleotide primer complementary to the template at that region. The oligonucleotide primer is extended using DNA polymerase in the presence of the four deoxynucleotide bases (DNA building blocks), along with a low concentration of a chain terminating nucleotide (most commonly a di-deoxynucleotide). Limited incorporation of the chain terminating nucleotide by the DNA polymerase results in a series of related DNA fragments that are terminated only at positions where that particular nucleotide is present. The fragments are then size-separated by electrophoresis a polyacrylamide gel, or in a narrow glass tube (capillary) filled with a viscous polymer. An alternative to using a labeled primer is to use labeled terminators instead; this method is commonly called “dye terminator sequencing.”

“Pyrosequencing” is an array based method, which has been commercialized by 454 Life Sciences. In some embodiments of the array-based methods, single-stranded DNA is annealed to beads and amplified via EmPCR®. These DNA-bound beads are then placed into wells on a fiber-optic chip along with enzymes that produce light in the presence of ATP. When free nucleotides are washed over this chip, light is produced as the PCR amplification occurs and ATP is generated when nucleotides join with their complementary base pairs. Addition of one (or more) nucleotide(s) results in a reaction that generates a light signal that is recorded, such as by the charge coupled device (CCD) camera, within the instrument. The signal strength is proportional to the number of nucleotides, for example, homopolymer stretches, incorporated in a single nucleotide flow.

Discrete volume or discrete space: A container, receptacle, or other arbitrary defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a target molecule and a indexable nucleic acid identifier (for example nucleic acid barcode). By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in our through the discrete volume, while other material, such as target molecules, maybe maintained in the discrete volume or space. Typically, a discrete volume with include a fluid medium, (for example, an aqueous solution, an oil, a buffer, and/or a media capable of supporting cell growth) suitable for labeling of the target molecule with the indexable nucleic acid identifier under conditions that permit labeling, such. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the discreet volume is an aqueous droplet in a water-in-oil emulsion.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when there is a sufficient degree of complementarity to avoid non-specific binding of the oligonucleotide or analog to non-target sequences under conditions where specific binding is desired. Such binding is referred to as specific hybridization.

Isolated: An “isolated” biological component (such a nucleic acid) has been substantially separated or purified away from other biological components in the cell of the organism in which the component naturally occurs, for example, extra-chromatin DNA and RNA, proteins and organelles. The term also embraces nucleic acids and proteins prepared by recombinant expression in a host cell as well as chemically synthesized nucleic acids. It is understood that the term “isolated” does not imply that the biological component is free of trace contamination, and can include nucleic acid molecules that are at least 50% isolated, such as at least 75%, 80%, 90%, 95%, 98%, 99%, or even 100% isolated.

Isotopic analog: “Isotopic analog” refers to a molecule that differs from another molecule in the relative isotopic abundance of an atom it contains. For example, peptide sequences containing identical sequences of amino acids, but differing in the isotopic abundance of an atom, are isotopic analogs of each other. Similarly, covalent modification reagents that have identical structures but differing isotope content are isotopic analogs, which can be separately reacted with corresponding peptides (identical sequence, different source) to provide covalently modified peptides that are isotopic analogs of one another such as covalently modified sample and standard peptides. The term “isotopic analog” is a relative term that does necessarily not imply that the isotopic analog necessarily contains an isotope that is present in less or greater abundance in nature. For example, a mass tag containing a natural abundance of ¹²C and ¹³C is an isotopic analog of a corresponding mass tag having non-natural abundances of these isotopes, and vice versa.

Mass spectrometry: A method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation.

Nucleic acid (molecule or sequence): A deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major building blocks for polymeric nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major major building blocks for polymeric nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

In some examples, nucleotides include those nucleotides containing modified bases, modified sugar moieties, and modified phosphate backbones, for example as described in U.S. Pat. No. 5,866,336 to Nazarenko et al. Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carb oxypropyl) uracil, 2,6-diaminopurine and biotinylated analogs, amongst others. Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Nucleic acid barcode, barcode, unique molecular identifier, or UMI: A short sequence of nucleotides (for example, DNA or RNA) that is used as an identifier for an associated molecule, such as a target molecule and/or target nucleic acid. A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes and/or UMIs can be attached, or “tagged,” to a target molecule and/or target nucleic acid. This attachment can be direct (for example, covalent or noncovalent binding of the barcode to the target molecule) or indirect (for example, via an additional molecule, for example, a specific binding agent, such as an antibody (or other protein) or a barcode receiving adaptor (or other nucleic acid molecule)). Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

Nucleic acid capture sequence: A nucleic acid sequence that specifically binds another nucleic acid, such as target nucleic acids and/or a barcode, such as a origin-specific barcode, or target molecule identification barcode and the like. A nucleic acid capture sequence (for example, a DNA or RNA molecule) may recognize a target nucleic acid molecule (for example, a DNA or RNA molecule) by hybridization or base-pairing interactions. Such capture sequence can be a single-stranded or have an overhang portion including nucleic acid sequences that can hybridize to sequences of target nucleic acid molecules. In some instances, a nucleic acid capture sequence can be attached to a nucleic acid barcode, for example by ligation, or by synthesizing both the nucleic acid specific binding agent and barcode as a single, contiguous nucleic acid. The length of sequences required for hybridization can vary depending, for example, on nucleotide content and conditions used but, in general, can be at least 4, 8, 12, 16, 20, 25, 30, 40, 50, 75, or 100 nucleotides in length.

Peptide barcode: A sequence of amino acids that can have a length of at least, for example, 1, 2, 3, 4, 5, 6, 7, 8, 9, 10, 11, 12, 13, 14, 15, 16, 17, 18, 19, 20, 21, 22, 23, 24, 25, 30, 40, 50, 75, or 100 amino acids. A specific peptide barcode can be distinguished from other peptide barcodes by having a different length, sequence, or other physical property (for example, hydrophobicity). In some examples, a peptide barcode is part of, or attached to a polypeptide identifier element.

Peptide linker: Short amino acid sequences that are typically flexible permitting the attachment of two different polypeptides (such as proteins, protein domains, and affinity tags), without disruption of the structure, aggregation (multimerization) or activity of the polypeptide components. Typically, a linear linking peptide consists of between two and 25 amino acids, although longer linkers are contemplated. Examples of Usually, the linear linking peptide is between two and 15 amino acids in length.

Polypeptide identifier element: A target molecule tag that is a polypeptide, and is attached to a target molecule (for example, a polypeptide target molecule), such that the polypeptide identifier element can be distinguished from other polypeptide identifier elements (for example, by a distinct length, amino acid sequence, or epitope within the) polypeptide identifier elements, for example, within the same discrete volume, or fraction, etc. In each instance that a “target molecule tag” is mentioned herein, the “target molecule tag” can be a “polypeptide identifier element,” unless indicated otherwise. Polypeptide identifier elements may optionally be encoded within the same nucleic acid molecule as a target polypeptide, in which case they can be referred to as “encoded polypeptide identifier elements” or “encoded peptide tags.” Polypeptide identifiers may be made of multiple component parts. In certain example embodiments, the polypeptide identifier may comprise one or more epitopes or affinity tags recognized by peptide binding molecules such as antibodies. In certain other example embodiments, the polypeptide identifier may comprise one or more peptide barcodes. The peptide barcode comprise a variable peptide sequence having at least one physically separable property.

A polypeptide identifier element can include one or more of the following: (i) an affinity tag (for example, hemagglutinin (HA) tags, Myc tags, FLAG tags, V5 tags, chitin binding protein (CBP) tags, maltose-binding protein (MBP) tags, GST tags, poly-His tags, and fluorescent proteins (for example, green fluorescent protein (GFP), yellow fluorescent protein (YFP), cyan fluorescent protein (CFP), dsRed, mCherry, Kaede, Kindling, and derivatives thereof)), (ii) a peptide barcode (see below), and/or (iii) a cleavage site. If an affinity tag is encoded within the same nucleic acid molecule as a target polypeptide, it can be referred to as an “encoded affinity tag.” Similarly, when a peptide barcode is encoded within the same nucleic acid molecule as a target polypeptide, it can be referred to as an “encoded peptide barcode.” A polypeptide identifier element can be comprised of sequences that have features of both affinity tags and peptide barcodes, as described herein. In certain embodiments, a polypeptide identifier element includes, in order, a cleavage site, a peptide barcode, and either an affinity tag or a fluorescent protein. In alternative embodiments, the positions of the peptide barcode and affinity tag are reversed. In particular embodiments, a polypeptide identifier element is fused to, for example, the C-terminus of a target polypeptide molecule to form a fusion protein.

A polypeptide identifier element including a particular affinity tag may be distinguished from other such polypeptide identifier elements within or from the same discrete volume or fraction, etc., by the length or other property of a peptide barcode that is also within the polypeptide identifier element, after release of the polypeptide identifier element from the target molecule (for example, by HPLC). Alternatively, different polypeptide identifier elements (for example, within a single discrete volume) can be distinguished from one another on the basis of their including both different affinity tags and different peptide barcodes.

Predictable mass difference: a predictable mass difference is a difference in the molecular mass of two molecules or ions (such as two peptides, peptide ions) that can be calculated from the molecular formulas and isotopic contents of the two molecules or ions. Although predictable mass differences exist between molecules or ions of differing molecular formulas, they also can exist between two molecules or ions that have the same molecular formula but include different isotopes of their constituent atoms. A predictable mass difference is present between two molecules or ions of the same formula when a known number of atoms of one or more type in one molecule or ion are replaced by lighter or heavier isotopes of those atoms in the other molecule or ion. For example, replacement of a ¹²C atom in a molecule with a ¹³C atom (or vice versa) provides a predictable mass difference of about 1 atomic mass unit (amu), replacement of a ¹⁴N atom with a ¹⁵N atom (or vice versa) provides a predictable mass difference of about 1 amu, and replacement of a ¹H atom with a ²H (or vice versa) provides a predictable mass difference of about 1 amu. Such differences between the masses of particular atoms in two different molecules or ions are summed over all of the atoms in the two molecules or ions to provide a predictable mass difference between the two molecules or ions. Thus, for example, if two molecules have the formula C₆H₆, where one molecule includes 6 ¹³C atoms and the other includes 6 ¹²C atoms, the predictable mass difference between the two molecules is about 6 amu (1 amu difference/carbon atom).

Primers: Short nucleic acid molecules, such as a DNA oligonucleotide, for example sequences of at least 15 nucleotides, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule, wherein the sequence of the primer is specific for the target nucleic acid molecule, for example so that the primer will hybridize to the target nucleic acid molecule under very high stringency hybridization conditions. The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50 or more consecutive nucleotides.

In particular examples, a primer is at least 15 nucleotides in length, such as at least 15 contiguous nucleotides complementary to a target nucleic acid molecule. Particular lengths of primers that can be used to practice the methods of the present disclosure, include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 15-60 nucleotides, 15-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, © 1991, Whitehead Institute for Biomedical Research, Cambridge, Mass.).

Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences. In one example, a primer includes a label.

Probe: An isolated nucleic acid capable of hybridizing to a specific nucleic acid (such as a nucleic acid barcode or target nucleic acid). A detectable label or reporter molecule can be attached to a probe. Typical labels include radioactive isotopes, enzyme substrates, co-factors, ligands, chemiluminescent or fluorescent agents, haptens, and enzymes. In some example, a probe is used to isolate and/or detect a specific nucleic acid.

Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

Probes are generally about 15 nucleotides in length to about 160 nucleotides in length, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160 contiguous nucleotides complementary to the specific nucleic acid molecule, such as 50-140 nucleotides, 75-150 nucleotides, 60-70 nucleotides, 30-130 nucleotides, 20-60 nucleotides, 20-50 nucleotides, 20-40 nucleotides, or 20-30 nucleotides.

Protein cleavage agent: An agent that cleaves a polypeptide or protein into smaller fragments. Protein cleavage agents include biological agents (such as proteolytic enzymes) and chemical protein cleavage agents (such as cyanogen bromide). Typically, but not always, protein cleavage agents cleave peptides and proteins at specific peptide bonds between pairs of particular amino acids. Where specific bonds are cleaved by a protein cleavage agent, the bonds that are cleaved are referred to as “protein cleavage agent sites.” Examples of proteolytic enzymes include endoproteases such as trypsin, chymotrypsin, endoprotease ArgC, endoprotease aspN, endoprotease gluC, and endoprotease lysC, and TEV endoprotease. Examples of chemical protein cleavage agents include cyanogen bromide, formic acid, and thiotrifluoroacetic acid. The specific bonds cleaved by an endoprotease or a chemical protein cleavage agents may be more specifically referred to as “endoprotease cleavage sites” and “chemical protein cleavage agent sites,” respectively.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166±1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15±20*100=75).

Specific Binding Agent: An agent that binds substantially or preferentially only to a defined target such as a polypeptide protein, enzyme, polysaccharide, oligonucleotide, DNA, RNA, recombinant vector or a small molecule. In an example, a “capture moiety specific binding agent” is capable of binding to a capture moiety that is linked to a nucleic acid, such as a nucleic acid barcode.

A nucleic acid-specific binding agent binds substantially only to the defined nucleic acid, such as RNA, or to a specific region within the nucleic acid. In some embodiments a specific binding agent is a nucleic acid barcode, that specifically binds to a target nucleic acid of interest.

A protein-specific binding agent binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Support: A solid or semisolid substrate to which something can be attached, such as a nucleic acid barcode, for example an origin-specific barcode. The attachment can be a removable attachment. Non-limiting examples of a support useful in the methods of the disclosure include a hydrogel, cell, bead, column, filter, slide surface, or interior wall of a discrete volume, such as a well in a microtiter plate, or vessel. In certain embodiments, the support is a hydrogel (such as a hydrogel bead) to which one or more origin-specific barcodes is coupled. A origin-specific barcodes reversibly coupled to a support can be detached from the support, for example enzymatic cleavage of a cleavage site on the origin-specific barcode. A support may be present in a discrete volume as set forth herein. In certain embodiments, the support is a hydrogel bead present in an emulsion droplet.

Target Molecule: A molecule, or in some examples a molecular complex, about which information is desired, for example origin, expression, type and the like. In some embodiments, a target molecule is labeled according to the methods disclosed herein. Examples of target molecules include, but are not limited to, peptides, polypeptides, proteins, antibodies, antibody fragments, amino acids, nucleic acids (such as RNA and DNA), nucleotides, carbohydrates, polysaccharides, lipids, small molecules, organic molecules, inorganic molecules, and complexes thereof. In some examples plurality of target molecules can be present in a sample, such as a sample that has been separated into a discrete volume or space (for example, multiple copies of the same target molecule or more than one distinct target molecule). In certain embodiments, a target nucleic acid molecule (for example, an RNA molecule) encodes a polypeptide target molecule (for example, a protein) present in the discrete volume. In particular embodiments, the polypeptide target molecule and the nucleic acid target molecule are expressed by a cell or cell-free expression system present in a discrete volume or space.

A target molecule can be bound by a specific binding agent associated with a nucleic acid barcode, such that the target molecule is labeled with the nucleic acid barcode, for examples a origin-specific barcode and/or a target molecule specific barcode. A plurality of target molecules, for example multiple copies of the same target molecule or multiple copies of more than one distinct target molecule, can be present in a discrete volume and labeled. In certain embodiments, a target nucleic acid molecule (such as a DNA or an RNA molecule) encodes a target molecule, such as a target protein present in the same discreet volume, and the target nucleic acid molecule and the polypeptide target molecule are labeled with the same barcode or matching barcodes or barcodes pre-identified as corresponding to one another, such as origin-specific nucleic acid barcodes. In particular embodiments, a target molecule and a target nucleic acid molecule are expressed by a cell or cell-free expression system present in a discreet volume.

Target nucleic acid molecule: Any nucleic acid present or thought to be present in a sample about which information would like to be obtained, such as its presence or absence and/or the molecules it interacts with, such as nucleic acids and proteins, either directly or indirectly. In some embodiments, a target nucleic acid of interest is a RNA, such as an mRNA, for example an mRNA encoding a target molecule. In some embodiments, a target nucleic acid of interest is a DNA.

Target molecule tag: A molecule (for example, a polypeptide, nucleic acid, or other) attached to a target molecule as described herein, and can be used in various approaches to identify the target molecule.

Under conditions that permit binding: A phrase used to describe any environment that permits the desired activity, for example conditions under which two or more molecules, such as nucleic acid molecules and/or protein molecules, can bind.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### II. Description of Several Embodiments

### A Introduction

One of the technically difficult areas of modern biological science and in particular proteomic analysis is how to meaningfully analyze multiple samples or even single cells in quick and cost efficient manner. Typically, proteomic analysis was done by mass spec which can be hampered by the cost of the mass spectrometers as well as the multiplex limitations due to the relatively small number of labeling schemes. The present disclosure solves those problems, and other, by providing multiplex proteomic analysis by sequencing. By combining proteomic analysis with next generation sequencing technologies, the inventors have greatly reduced the cost associated with the analysis of multiple samples as well as increased the efficiency of detection. Thus, this disclosure provides methods and compositions for high-throughput labeling of target molecules, or target molecule tags (for example, polypeptide identifier elements, such as affinity tags and/or peptide barcodes), with specific nucleic acid barcodes (for example, origin-specific barcodes). The presence, quantity, and/or activity of a target molecule, or multiple target molecules, in a particular discrete volume can be determined, for example after pooling (for example polling of a plurality of discrete volume with different nucleic acid barcodes) and determining the sequence (either directly or indirectly) of the origin-specific nucleic acid barcodes. In addition to the origin-specific nucleic acid barcodes, target molecules and/or target molecule tags can be labeled with additional nucleic acid barcodes (optionally in the form of a nucleic acid barcode concatemer) in a specific manner based on any of a number of different properties of the target molecules or the target molecule tags. The ability to add multiple barcodes (both nucleic acid and peptide) facilitates further levels of characterization, greatly increasing the ability and power of multiplexing. The present disclosure thus enables highly complex combinatorial analyses by associating information about different target molecules (and/or associated target molecule tags), their sources (for example, discrete volumes in which they are made or labeled), physical characteristics (for example, length, affinity, and/or hydrophobicity), and/or different treatment conditions with distinct nucleic acid barcodes, which can be de-convoluted from pooled samples using high-throughput sequence analysis, such as high-throughput sequencing technologies. Moreover, in some examples, the methods of the disclosure involve simultaneous co-labeling of target polypeptides and DNA or RNA molecules of interest (for example, DNA and/or RNA encoding the co-labeled target polypeptides, or DNA and/or RNA of a reporter cell line) with the same or matching origin-specific nucleic acid barcodes, thus enabling, for example, coupling of a genotype, to a phenotype of interest, for example the expression of specific target molecules and their expression levels. The methods of the disclosure can thus be used in the characterization of gene expression systems in the form of, for example, multi-part DNA assemblies, in massively multiplex form, and provide a cornerstone for the progress of synthetic biology.

### B. Methods

Disclosed herein is a method of multiplex proteomic analysis using nucleic acid identifiers to tag and identify target molecules, such as peptides, for example proteins in a sample or set of samples. The methods include assigning a set of target molecules (such as polypeptides, for example proteins) to specific discrete volumes, while maintaining information about the origin of the target molecules. The disclosed methods include the labeling of target molecules present in the discrete volume with distinct nucleic acid barcodes (for example an origin-specific barcode) associated so the origin of the molecules within, or labeled within the individual discrete volumes, can be determined at a later time, for example during proteomic analysis by sequencing.

Optionally, a peptide identifier element is linked to the target polypeptides to allow multiplex characterization of the presence and/or amount of the individual polypeptides of interest. Because the specific target molecules from each discrete volume are labeled with specific nucleic acid barcodes, the individual discrete volumes can be pooled and analyzed together, greatly increasing throughput while minimizing sample processing variations and significantly reducing the cost associated with such analysis.

In some embodiments, the method includes labeling the target polypeptides in with distinguishable mass tags present in the discrete volumes to create mass tag labeled target polypeptides, wherein the mass tag labeled target polypeptides comprises a mass tag matched to the target polypeptide and/or mass tags matched to the peptide identifier element; and detecting the mass tags, thereby detecting the target polypeptides.

The disclosed method includes providing a sample of interest that includes cells of interest (for example cells that have differences in the expression of proteins), or an acellular system (such as a cell-free extract or a cell-free transcription and/or cell-free translation mixture). The cells (such as single cells) or a portion of the acellular system are segregated into individual discrete volumes that include an origin-specific barcode that includes a unique nucleic acid identification sequence that maintains or carries information about the origin of the cell, or acellular system, in the sample. Target molecules in the discrete volumes are labeled with the origin-specific barcodes present in the discrete volumes to create origin-labeled target molecules, wherein the origin-labeled target molecules from each individual discrete volume include the same, or matched, unique indexing nucleic acid identification sequence. The nucleotide sequence of the origin-specific barcodes is detected to assign the set of target molecules to a specific discrete volume.

A target molecule and/or target nucleic acid can be labeled with a nucleic acid barcode that is already present upon introduction into, or production of, the target molecule, in the discrete volume. In certain embodiments, the methods further include assigning the set of target molecules to target nucleic acids (such as RNA, for example mRNA or DNA, for example cDNA) in the sample or set of samples while maintaining information about sample origin of the target molecules and target nucleic acids. In this embodiment, the target nucleic acids in the discrete volumes are labeled with the origin-specific barcodes present in the discrete volumes to create origin-labeled target nucleic acids, wherein the origin-labeled target nucleic acids from each discrete volume include the same, or matched, unique indexing nucleic acid identification sequence as the origin-labeled target molecules. The nucleotide sequence of the origin-specific barcodes are detected, thereby assigning the set of target molecules to target nucleic acids in the sample set of samples while maintaining information about sample origin of the target molecules and the target nucleic acids. In some examples of the method, the target nucleic acids encode the target molecules, such as target polypeptides, for example target proteins. The sequence of the origin-specific barcode, amongst other sequences (such as other nucleic acid barcodes and/or coding sequencing, for example target nucleic acid sequences), can be detected by any method known in the art, such as by amplification, sequencing, hybridization and any combination thereof. In some embodiments, the discrete volumes are pooled to create a pooled sample, for example so that the analysis can be carried out in a multiplex fashion.

In some embodiments, the one or more target molecules are linked to a molecule identifier element. In certain embodiment, molecule identifier elements within a single discrete volume comprise distinct molecule identifier elements, differentiated from the molecule identifier elements in other discrete volumes. In certain embodiments, the target molecules are target polypeptides, such as target proteins, and the molecule identifier element includes a polypeptide identifier element. In some examples, the target polypeptide is covalently linked to the polypeptide identifier element, for example covalently linked by a peptide linker, which optionally includes a peptide cleavage site, for example a site of chemical cleavage, and/or enzymatic cleavage. Peptide cleavage sites, and method and reagents for peptide cleavage, are known to those of ordinary skill in the art.

In some embodiments, the polypeptide identifier elements include affinity tags, such as hemagglutinin (HA) tags, Myc tags, FLAG tags, V5 tags, chitin binding protein (CBP) tags, maltose-binding protein (MBP) tags, GST tags, poly-His tags, and fluorescent proteins (for example, green fluorescent protein (GFP), yellow fluorescent protein (YFP), cyan fluorescent protein (CFP), dsRed, mCherry, Kaede, Kindling, and derivatives thereof, FLAG tags, Myc tags, AU1 tags, T7 tags, OLLAS tags, Glu-Glu tags, VSV tags, or a combination thereof. Other Affinity tags are well known in the art. Such labels can be detected and/or isolated using methods known in the art (for example, by using specific binding agents, such as antibodies, that recognize a particular affinity tag). Such specific binding agents (for example, antibodies) can further contain, for example, detectable labels, such as isotope labels and/or nucleic acid barcodes such as those described herein.

In specific embodiments, different target molecules are linked to different affinity tags, for example so that the different target molecules can be labeled, isolated, or otherwise analyzed based on the presence of the affinity tags. In other specific examples, different target molecules include identical affinity tags. In specific embodiments, some different target molecules include the same affinity tag and some different molecules include different affinity tags. Such differential labeling of target molecules with such affinity tags when combined with peptide barcodes increases the number of unique tagging elements even with a limited number of different affinity tags. Thus in some embodiments, the different target molecules in the sample include sets of different target molecules and the different sets of target molecules in the sample include different affinity tags. In some examples, the target molecules are linked to between about 2 and about 200 different affinity tags, such as 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 (or maybe more) different affinity tags, such as between about 2 and about 20, about 5 and about 50, about 35 and about 150, about 75 and about 200, and about 50 and about 150 different affinity tags.

In some embodiments, a polypeptide identifier element further includes a peptide barcode, such as different peptide barcodes, which can have different physical characteristics (amino acid sequence, sequence length, charge, size, molecular weight, hydrophobicity, reverse phase separation, affinity or other separable property), allowing separation of the different peptide barcodes. In certain embodiments, a plurality of encoded polypeptide identifier elements are distinguished from one another using chromatography methods. In specific examples, high performance liquid chromatography (HPLC), is used to separate the encoded polypeptide identifier elements, for example by their peptide barcode length and/or their hydrophobicity. Each resultant HPLC fraction can be associated with a distinct HPLC fraction-specific barcode, which can be added to the fraction and, for example, ligated to an origin-specific barcode and/or an affinity tag-specific barcode, optionally in the form of a nucleic acid barcode concatemer. In certain embodiments, a plurality of encoded polypeptide identifier elements can be separated from one another by affinity to a plurality of specific binding agents (for example, antibodies), which can be washed into and out of a mixture containing the plurality of encoded polypeptide identifier elements (for example, one specific binding agent at a time), thereby pulling out fractions containing the encoded polypeptide identifier elements that bind to specific binding agent.

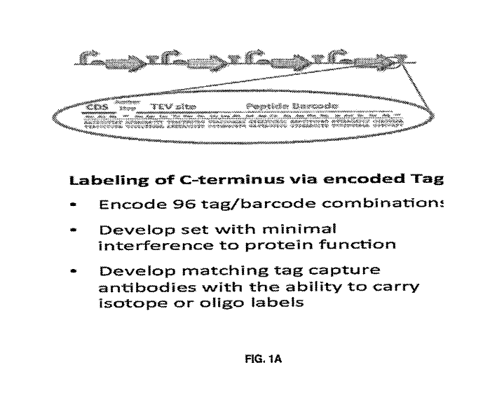

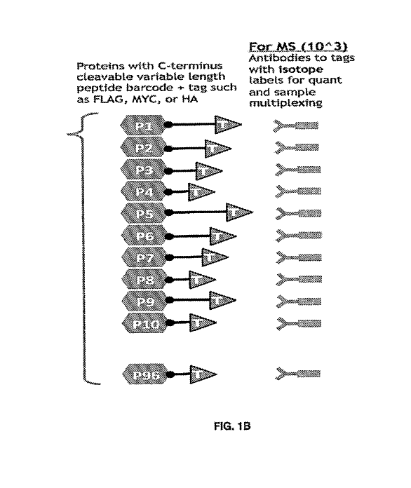

Encoded polypeptide identifier elements can be added to a target molecule or specific-binding agent using recombinant DNA technology known in the art. In certain embodiments, an encoded polypeptide identifier element is expressed as a fusion protein with a target molecule or specific-binding agent of interest, for example, by engineering an expression construct in which the nucleic acid sequence encoding the polypeptide identifier element is incorporated at the N- or C-terminal end of the coding sequence (CDS) of the target molecule or specific-binding agent. For example, the expression construct may include a nucleic acid sequence encoding the polypeptide identifier element positioned upstream or downstream of the target molecule CDS. The expression construct may also include a promoter, terminator, ribosome binding site, and/or other genetic elements known in the art to be useful for controlling the expression of a protein, for example, the target molecule-encoded polypeptide identifier element fusion protein. Nucleic acid sequences encoding multiple target molecule-encoded polypeptide identifier element fusion proteins can be included in a single expression construct, for example, by including multiple genes, one per target molecule-encoded polypeptide identifier element, for example, as shown in FIG. 1. In certain embodiments, the target polypeptide, optionally the peptide linker, and the polypeptide identifier element are encoded by a single nucleic acid sequence, such as a single target nucleic acid sequence, operatively connected to a promoter. In specific embodiments, the polypeptide identifier element is located C-terminal to the target polypeptide. A cleavage site can be positioned, for example, between an encoded polypeptide identifier element and the target molecule, or within an encoded polypeptide identifier element, such that the encoded polypeptide identifier element can be removed from the target molecule or specific-binding agent by cleavage, for example, by a protease. In specific examples, peptide cleavage site is positioned between the target polypeptide and the polypeptide identifier element. In some embodiments, target polypeptide and the polypeptide identifier element, and optionally the peptide linker, are contained in a single polypeptide. In certain embodiments, the encoded polypeptide identifier element further includes a peptide moiety that can be bound by a further specific-binding agent.

In some embodiments, a polypeptide identifier element includes two or more different peptide barcodes, such as about 2 and about 200 different peptide barcodes. In some examples, the target molecules are linked to between 2 and about 200 different peptide barcodes, such as 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, or 200 (or maybe more) different affinity tags, such as between about 2 and about 20, about 5 and about 50, about 35 and about 150, about 75 and about 200, and about 50 and about 150 different affinity tags. In specific embodiments, a polypeptide identifier element includes an affinity tag and a peptide barcode. In some embodiments, such polypeptide identifier elements can be combined with other peptide labels (for example, affinity tags or other encoded polypeptide identifier elements) to increase the possible number of combinations of distinct labels that can be associated with a target molecule. An encoded polypeptide identifier element can, for example, incorporate one or more peptide labels (for example, an affinity tag or fluorescent protein as described herein). An affinity tagged encoded polypeptide identifier element can be bound, for example, by an antibody specific to the affinity tag. For example, the antibody can have an affinity tag-specific isotope label, which can be identified by mass spectrometry, or an antibody conjugated to an affinity tag-specific oligonucleotide barcode, which can be identified by cleaving off the barcode and determining the sequence. The affinity tag-specific barcode can be ligated to a compartment-specific barcode, for example, before or after cleavage from the antibody. In certain embodiments, the affinity tagged encoded polypeptide identifier elements are further separated by their HPLC signatures (for example, separated according to linker length and/or hydrophobicity using HPLC) and the affinity tag-specific barcode is further attached to an HPLC fraction-specific barcode. In some embodiments of the method, the pooled sample, or an enriched fraction thereof, is fractionated based on one or more properties of the peptide barcodes and isolating the fractions of interest, for example barcode length or hydrophobicity. In some embodiments, the fractionation includes the use of chromatography. In specific embodiments the chromatography comprises HPLC separation. The affinity tag-specific barcode can be ligated to both a compartment-specific barcode and an HPLC fraction-specific barcode. This three-barcode concatemer can then be isolated, pooled with other barcode concatemers, and sequenced to determine the presence of a particular target molecule (identifiable by its unique combination of encoded polypeptide identifier element HPLC signature and affinity tag) in a particular compartment. A polypeptide identifier element incorporating a peptide label can thus be distinguished by the length of its linker region and/or by its peptide label. For example, an encoded polypeptide identifier element containing a linker region and a FLAG tag can be used to tag a target molecule (for example, by producing a fusion protein of the target molecule and the encoded polypeptide identifier element), with a cleavage site positioned on the polypeptide identifier element such that cleavage results in release of the linker region and FLAG tag from the target molecule. The encoded polypeptide identifier element can then be isolated from the mixture, for example, by immunoprecipitation using, for example, an anti-FLAG antibody. If a plurality of such encoded polypeptide identifier elements are pooled prior to the immunoprecipitation, distinct polypeptide identifier elements can be distinguished and/or isolated by the lengths of their linker regions.

In some embodiments isolating the labeled target molecules includes performing liquid chromatography-mass spectrometry (LC-MS) on the pool, or a partially purified fraction thereof, and isolating one or more peaks corresponding to one or more labeled target molecules to be isolated. In some embodiments, each of the sample-specific barcodes is designed to shift the peak associated with a target molecule in a mass spectrometry profile by a predictable mass different corresponding to different isotopes of a particular atom or atoms, or a different peptide tag. In some embodiments, the origin-specific barcode is cleaved from the isolated labeled target molecules.

In some embodiments, one or more of the polypeptide identifier element, the affinity tag, or the peptide barcode comprises a C-terminal cysteine. In some embodiments, the polypeptide identifier element is cleaved from the target molecule. In some embodiments, the affinity tag is labeled with an affinity tag-specific nucleic acid barcode. In some examples, labeling the affinity tag with the affinity tag-specific nucleic acid barcode includes contacting the affinity tag with an affinity tag specific binding agent (such as an antibody), that specifically binds the affinity tag, wherein affinity tag specific binding agent includes the affinity tag-specific nucleic acid barcode. In some embodiments, the affinity tag specific binding agent includes an antibody. In some embodiments of the method, an origin-specific barcode is attached to the affinity tag-specific nucleic acid barcode.

An encoded polypeptide identifier element may include a protein G moiety, for example, tethered to one end of an affinity tag or a linker region. Thus, the encoded polypeptide identifier element can be isolated or enriched using, for example, an antibody column, for example, prior to cleavage of an attached nucleic acid identifier or after a set of binding moieties recognizing the affinity tag are bound to the encoded polypeptide identifier element. In some embodiments, the method includes enriching for the polypeptide identifier elements, for example after cleavage from the target molecule. In specific embodiments, enriching for the polypeptide identifier elements include contacting the polypeptide identifier element with immobilized protein G, such as a protein G column or the solid or semisolid support, such as beads.

In certain example embodiments, target polypeptides are labeled with a target polypeptide specific barcode. In some embodiments labeling a target polypeptide with the target polypeptide specific barcode includes contacting the target polypeptide with a target polypeptide specific binding agent, that specifically binds the target polypeptide, wherein the target polypeptide specific binding agent comprises the target polypeptide specific nucleic acid barcode. In some embodiments, the target polypeptide specific binding agent comprises an antibody. In some embodiments, the method further includes attaching an origin-specific barcode to the target polypeptide specific nucleic acid barcode.

In some embodiments, the peptide barcode is labeled with a peptide barcode-specific nucleic acid barcode. In some embodiments, labeling the peptide barcode comprises with a peptide barcode-specific nucleic acid barcode includes the sample with a peptide barcode specific binding agent that specifically binds the peptide barcode, wherein the peptide barcode specific binding agent includes a peptide barcode-specific nucleic acid barcode. In some embodiments, the origin-specific barcode is attached to the peptide barcode-specific nucleic acid barcode, for example as a concatemer. In some examples, the sequence of the peptide barcode-specific nucleic acid barcode is correlated to the length of the peptide barcode, this is specific peptide-specific nucleic acid barcodes can be assigned to specific lengths and/or compositions of the peptide barcode.

In some embodiment, the labeled target molecules are isolated, for example from the pooled and/or fractionated sample, or even prior to pooling. In some examples, isolating a population of target molecules is based on one or more properties of one or more of the target polypeptides. In some embodiment, the target molecules, optionally in association and the target nucleic acids are produced by the individual cells in the discrete volumes. In some examples the cells are wild-type cells. In some embodiments each of the polypeptides comprises a cysteine residue. In some embodiments, origin-specific nucleic acid barcodes are attached to the cysteine residues.

In some embodiments, the method is used to label a set of target molecules that are nucleic acids from a sample, such as the amplicons from a cell, set or cells, or an acellular system. In such a method, the nucleic acids from a single discrete volume are labeled with a specific origin-specific barcode, while nucleic acids from another, or a plurality of other discrete volumes are labeled with different origin-specific barcodes, allowing for multiplex analysis of the nucleic acids, for example to study gene expression difference in the individual discrete volumes, for example when exposed to different conditions, or being of different cell types.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, a origin-specific barcode may be a nucleic acid including a barcode and a hybridization site for a specific primer. In particular embodiments, a set of origin-specific barcode includes a unique primer specific barcode made, for example, using a randomized oligo type NNNNNNNNNNN.

Unique molecular identifiers are a subtype of nucleic acid barcode that can be used, for example, to normalize samples for variable amplification efficiency. For example, in various embodiments, featuring a solid or semisolid support (for example a hydrogel bead), to which nucleic acid barcodes (for example a plurality of barcode sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcode (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprise a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

In some embodiments of the disclosed methods, determining the identity of a nucleic acid, such as a nucleic acid barcode, includes detection by nucleic acid hybridization. Nucleic acid hybridization involves providing a probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (for example, low temperature and/or high salt) hybrid duplexes (for example, DNA:DNA, RNA:RNA, or RNA:DNA) will form even where the annealed sequences are not perfectly complementary. Thus, specificity of hybridization is reduced at lower stringency. Conversely, at higher stringency (for example, higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. In some examples, RNA is detected using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992).

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (such as fluorescein-labeled UTP and/or CTP) incorporates a label into the transcribed nucleic acids.

Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241.

Means of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a specific-binding agent that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

In some embodiments, labeling of the target molecule includes directly attaching the origin-specific barcode to the target molecule. In some embodiments, labeling of the target molecule includes attaching the origin-specific barcode to the target molecule indirectly. Indirect attachment includes binding a target molecule specific binding agent to the target molecule, where the target molecule specific binding agent is indirectly or directly attached to the origin-specific barcode. In certain embodiments, a specific binding agent is an antibody, such as a whole antibody or an antibody fragment, such as an antigen-binding fragment. A specific binding agent can alternatively be a protein or polypeptide that is not an antibody. A specific binding agent may thus be, for example, a kinase, a phosphatase, a proteasomal protein, a protein chaperone, a receptor (for example, an innate immune receptor or signaling peptide receptor), a synbody, an artificial antibody, a protein having a thioredoxin fold (for example, a disulfide isomerase, DsbA, glutaredoxin, glutathione S-transferase, calsequestrin, glutathione peroxidase, or glutathione peroxiredoxin), a protein having a fold derived from a thioredoxin fold, a repeat protein, a protein known to participate in a protein complex, a protein known in the art as a protein capable of participating in a protein-protein interaction, or any variant thereof (for example, a variant that modifies the structure or binding properties thereof). A specific binding agent can be any protein or polypeptide having a protein binding domain known in the art, including any natural or synthetic protein that includes a protein binding domain. A specific binding agent can also be any protein or polypeptide having a polynucleotide binding domain known in the art, including any natural or synthetic protein that includes a polynucleotide binding domain. In some instances, a specific binding agent is a recombinant specific binding agent.

A specific binding agent (for example, an antibody) can, for example, be attached to a nucleic acid barcode (for example, an origin-specific barcode). For example, the specific binding agent may include a cysteine residue that can be attached to a nucleic acid barcode. In other instances, a specific-binding agent can be a nucleic acid attached to a barcode. A specific binding agent attached to a nucleic acid barcode can recognize a target molecule of interest. A nucleic acid barcode may identify a specific binding agent as recognizing a particular target molecule of interest. A nucleic acid barcode may be cleavable from a specific binding agent, for example, after the specific binding agent has bound to a target molecule.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

In some embodiments, the target molecule comprises a target polypeptide and the specific binding agent that specifically binds to the target molecule in the sample comprises a polypeptide specific binding agent that specifically binds to a target polypeptide. In some embodiments, the polypeptide specific binding agent comprises an antibody, or fragment thereof and/or a protein-binding domain or fragment thereof, or a nucleic acid sequence that specifically binds to the target polypeptide, for example if the target polypeptide includes a nucleic acid binding domain. In some embodiments the target molecule specific binding agent specifically binds both the target molecule and the origin-specific barcode. In some examples, the target molecule is incubated with the target molecule specific binding agent that bind both the target molecules and the origin-specific barcode and the target molecule specific binding agents are not bound to the target molecule and/or origin-specific barcode are removed prior to segregation into discrete volumes. In some examples, the target molecule specific binding agent comprises a target molecule specific binding agent barcode, encoding the identity of the target molecule specific binding agent. In some examples, the target molecule specific binding agent barcode can bind to the origin-specific barcode via base-pairing interactions. In specific examples, the origin-specific barcode is a primer for the synthesis of the complementary strand of the target molecule specific binding agent barcode. In some examples, the sequence of the target molecule specific binding agent barcode, amongst other sequences, is be detected. The sequence of the target molecule specific binding agent barcode can be detected by any method known in the art, such as by amplification, sequencing, hybridization and any combination thereof. In addition to origin-specific nucleic acid barcodes, target molecules can be labeled with additional nucleic acid barcodes (optionally in the form of a nucleic acid barcode concatemer) in a specific manner based on any of a number of different properties of the target molecules and/or conditions to which they are exposed, facilitating further levels of characterization. In still other embodiments, the target molecule is a polypeptide and it is directly labeled with a nucleic acid barcode, such as a target molecule specific binding agent and/or origin-specific barcode via encoded cysteine residues, for example, a C-terminal cysteine residue.

In particular examples, the sequence of the target molecule specific binding agent barcode is amplified, for example using PCR. A nucleic acid encoding an antigen or specific binding agent can be sub-cloned into an expression vector for protein production, for example, an expression vector for production of binding moieties in, for example, E. coli. Produced binding moieties may be purified, for example, by affinity chromatography. In embodiments in which a specific binding agent includes one or more fragments substantially similar to an antibody or antibody fragment, the fragments may be incorporated into known antibody frameworks for expression. For instance, if a specific binding agent is an scFv, the heavy and light chain sequences of the scFv may be cloned into a vector for expression of those chains within an IgG molecule.

In some embodiments, a target molecule comprises a target nucleic acid, such as a DNA or RNA and the specific binding agent that specifically binds to the target molecules in the sample comprises a nucleic acid sequence or nucleic acid binding domain that specifically binds, and/or hybridizes to the target nucleic acid. Target nucleic acids include RNA, such as mRNA, and DNA, such as cDNA. In some embodiments of the disclosed methods, cDNAs synthesized from the target nucleic acids, wherein the cDNA comprises the nucleic acid sequence of the target nucleic acid, or a fragment thereof and the sequence of the origin-specific barcode. In some example, the origin-specific barcodes are primers for the cDNA synthesis. In certain embodiments, the target nucleic acid, or complement thereof, encode a polypeptide of interest. In some embodiments, the target molecule comprises a target DNA and the specific binding agent that specifically binds to the target molecules in the sample comprises a nucleic acid sequence or DNA binding domain that specifically binds, and/or hybridizes to the target DNA.

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

A nucleic acid barcode may be cleavable from a specific binding agent, for example, after the specific binding agent has bound to a target molecule. In some embodiments, the origin-specific barcode further comprises one or more cleavage sites. In some example at least one cleavage site is oriented such that cleavage at that site releases the origin-specific barcode from a substrate, such as a bead, for example a hydrogel bead, to which it is coupled. In some examples at least one cleavage site is oriented such that the cleavage at the site releases the origin-specific barcode from the target molecule specific binding agent. In some examples, a cleavage site is a enzymatic cleavage site, such a endonuclease site present in a specific nucleic acid sequence. In other embodiments, a cleavage site is a peptide cleavage site, such that a particular enzyme can cleave the amino acid sequence. In still other embodiments, a cleavage site is site of chemical cleavage.

In some embodiments, each of the origin-specific barcodes comprises one or more indexes, one or more sequences that enable gene specific capture and/or amplification, and/or one or more sequences that enable sequencing library construction.

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

In some embodiments, the more than one target molecule specific binding agent is attached to nucleic acid barcodes, such as an origin-specific barcode amongst others, having the same sequence. In certain embodiments, the more than one target molecule specific binding agent is attached to nucleic acid barcodes having distinct sequences. In some instances, a plurality of target molecule specific binding agent can be added to a discrete volume. Alternatively, a plurality of target molecule specific binding agent can be added separately. Each different target molecule specific binding agent can optionally be, for example, associated with an experimental condition.

One of the superior properties of the disclosed methods is the samples, such as multiple discrete volumes, can be analyzed together in a single reaction, for example a pooled reaction. Thus, in some examples, the discrete volumes are pooled to create a pooled sample. The target molecules and/or target nucleic acids from a plurality discrete volumes, labeled according to the disclosed methods, can be combined to form a pool. For example, labeled target molecules and/or target nucleic acids in a plurality of emulsion droplets can be combined by breaking the emulsion. Thus, in some embodiments, the emulsion is broken. The pools can be comprised of labeled target molecules and/or target nucleic acids coming from a large number of discrete volumes (for example, at least 2, 3, 4, 5, 6, 7, 8, 9, 10, 20, 30, 40, 50, 100, 500, 1,000, 2,500, 5,000, 10,000, 50,000, 100,000, 500,000, 1,000,000, 2,000,000, or more; in various examples, for example, those utilizing discreet volumes in plates, the numbers can be, for example, at least 6, 24, 96, 192, 384, 1,536, 3,456, or 9,600), thus facilitating processing of very large numbers of samples at the same time (for example, by highly multiplexed affinity measurement), leading to great efficiencies.

Labeled target molecules and/or target nucleic acids can be isolated or separated from a pool. Exemplary isolation techniques include, without limitation, affinity capture, immunoprecipitation, chromatography (for example, size exclusion chromatography, hydrophobic interaction chromatography, reverse-phase chromatography, ion exchange chromatography, affinity chromatography, metal binding chromatography, immunoaffinity chromatography, high performance liquid chromatography (HPLC), and liquid chromatography-mass spectrometry (LC-MS)), electrophoresis, hybridization to a capture oligonucleotide, phenol-chloroform extraction, minicolumn purification, or ethanol or isopropanol precipitation. Chromatography methods are described in detail, for example, in Hedhammar et al. (“Chromatographic methods for protein purification,” Royal Institute of Technology, Stockholm, Sweden), which is incorporated herein by reference. Such techniques can utilize a capture molecule that recognizes the labeled target molecule or a barcode or specific-binding agent associated with the target molecule. For example, a target antibody can be isolated by affinity capture using protein G. Labeled target molecules can be further labeled with capture labels, such as biotin. A plurality of target molecules (for example, a plurality of identical target molecules, or a population of target molecules including multiple distinct target molecules) can be isolated simultaneously or separately.

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments the specific binding agent is has been immobilized for example on a solid support, thereby isolating the origin-specific barcode.

By “solid or semisolid support or carrier” is intended any support capable of binding an origin-specific barcode. Well-known supports or carriers include hydrogels, glass, polystyrene, polypropylene, polyethylene, dextran, nylon, amylases, natural and modified celluloses, polyacrylamides, agarose, gabbros and magnetite. The nature of the carrier can be either soluble to some extent or insoluble for the purposes of the present disclosure. The support material may have virtually any possible structural configuration so long as the coupled molecule is capable of binding to targeting probe. Thus, the support configuration may be spherical, as in a bead, or cylindrical, as in the inside surface of a test tube, or the external surface of a rod. Alternatively, the surface may be flat such as a sheet or test strip. Thus, some embodiments of the method include selectively isolating the origin-labeled molecules and origin-labeled nucleic acids, for example from a pooled sample. In some embodiment, the cells are lysed.

Target molecules, include any molecule present in a discrete volume about which information is desired, such as expression, activity, and the like. In certain embodiments, a target molecule that can be labeled and characterized according to the disclosed methods include polypeptides (such as but not limited to proteins, antibodies, antigens, immunogens, protein complexes, and peptides), which are in some cases modified, such as post-translationally modified, for example glycosylated, acetylated, amidated, formylated, gamma-carboxyglutamic acid hydroxylated, methylated, phosphorylated, sulfated, or modified with pyrrolidone carboxylic acid. Target molecules can be natural, recombinant, or synthetic. As disclosed herein a given discrete volume can include one or more distinct target molecules, meaning that several targets can be present in the discrete volume. In some instances, a discrete volume can include a target polypeptide molecule and a target nucleic acid molecule encoding the target polypeptide molecule, such that the nucleic acid sequence encoding the target polypeptide can be readily determined. Thus, in certain instances, a polypeptide target molecule and a nucleic acid target molecule are produced by the same cell. In particular instances, the polypeptide target molecule and the target nucleic acid are labeled with the same origin-specific barcodes or matching barcodes. In some examples, the target molecule is not encoded by a target nucleic acid. Target molecules can be expressed in cells or in extracts, such as cell free extracts. In some embodiments, the target molecule comprises a polypeptide, nucleic acid, polysaccharide, and/or small molecule. In specific embodiments, the polypeptide comprises an antibody, an antigen, or a fragment thereof. In specific examples, the target molecules represent a library of randomly mutated polypeptides. In specific embodiments, the target molecule is expressed on the surface of a cell, such as a cell surface protein, or a fragment thereof, such as a cell surface domain of a protein.

In certain example embodiments, the target molecules, optionally in association with target nucleic acids, are produced by individual cells in the individual discrete volumes. In certain example embodiments, the target molecules are polypeptides and the target nucleic acids encode the target molecules in their respective discrete volumes. In certain other example embodiments, the cells are B-cells and the target molecules are antibodies and the target nucleic acids encode the antibodies. In some embodiments, a target molecule is present on the surface of a cell. Thus, in some embodiments, a target molecule, such as a protein or polypeptide, is one that is normally found on the surface of a cell. In other embodiments, a target molecule, such as a protein or polypeptide, is not normally found on the surface of a cell, but is expressed on the surface of a cell, such as by recombinant means. In certain instances, a target molecule is normally found within a cell, such as in the cytoplasm or in an organelle. In these examples, it may be necessary to lyse a cell in which the target molecule is produced in order to label it. Protein or nucleic acid target molecules can be naturally produced by a cell or can be recombinantly produced based on, for example, the presence of a synthetic construct in a cell.

In some embodiments, a target molecule is a protein or peptide found in a protein or peptide database (for example, SWISS-PROT, TrEMBL, SBASE, PFAM, or others known in the art), or a fragment or variant thereof. A target molecule may be a protein or peptide that may be derived (for example, by transcription and/or translation) from a nucleic acid sequence known in the art, such as a nucleic acid sequence found in a nucleic acid database (for example, GenBank, TIGR, or others known in the art), or a fragment or variant thereof.

Target molecules such as polypeptides can optionally be produced by one or more synthetic multi-gene genetic constructs, which can be present in discreet volumes as described herein in a cell or with a cell-free extract. In specific embodiments, the one or more synthetic genetic constructs includes a set of synthetic genetic constructs, which optionally are comprised of combinatorially generated parts. In some embodiments, the one or more synthetic genetic constructs includes the peptide coding sequences for a biosynthetic pathway. In some embodiments, one or more synthetic genetic constructs includes the peptide coding sequences for a gene cluster. In specific embodiments, the cells are transformed or transfected with one or more synthetic genetic constructs. Such target molecules can be characterized according to the methods of the disclosure in the context of genetic construct prototyping, as described herein, which optionally can be a component of a synthetic biology scheme.

A target molecule may be a protein or polypeptide endogenous to an organism, such as a protein or polypeptide selectively expressed or displayed by one or more cells of an organism. For example, the protein or polypeptide may be a cell surface marker expressed by the one or more cells. The organism may be, for example, a eukaryote (for example, a mammal, such as a human), virus, bacterium, or fungus. In some embodiments, a plurality of distinct target molecules is selected from a single organism. In some embodiments, a plurality of distinct target molecules of the present disclosure is selected from a single organism. In alternate embodiments, a plurality of distinct target molecules of the present disclosure is selected from among a plurality of distinct organisms.

In various embodiments, a target molecule is displayed or expressed on the surface of a cell. Thus, in the case of proteins, the target molecule can be, for example, an antibody, cell surface receptor, signaling protein, transport protein, cell adhesion protein, enzyme, or a fragment thereof. Cell surface target molecules include known transmembrane proteins, i.e., a protein known to have one or more transmembrane domains, a protein previously identified as associating with the cellular membrane, or a protein predicted to have one or more transmembrane domains by one or more methods of domain prediction known in the art. The cell surface target molecule may alternatively be a fragment of such a protein. A cell surface target molecule can be an integral membrane protein or a peripheral membrane protein, and/or can be a protein or polypeptide having a sequence present in nature, substantially similar to a sequence present in nature, engineered or otherwise modified from a sequence present in nature, or artificially generated, for example, by techniques of molecular biology (for example, fusion proteins).

As disclosed herein, a polypeptide target molecule can contain or be attached to a target molecule tag, such as a polypeptide identifier element A polypeptide target molecule attached to an encoded polypeptide identifier element can be recognized by a specific-binding agent specific to the encoded polypeptide identifier element (for example, an antibody capable of recognizing an affinity tag located in the encoded polypeptide identifier element; see below). A polypeptide target molecule can be captured according to the methods of the present disclosure, for example, by use of a molecule (for example, an antibody) that binds to an epitope present in an encoded polypeptide identifier element attached to the polypeptide target molecule. A polypeptide target molecule can be fused to an encoded polypeptide identifier element, for example, by introducing an expression construct into a cell. The expression construct can include, for example, a plasmid, vector, artificial chromosome, and/or a transgene, and may contain the coding sequence of the target polypeptide and a sequence encoding the encoded polypeptide identifier element, for example, positioned upstream or downstream from the target polypeptide coding sequence, as well as elements needed to express the target polypeptide/encoded polypeptide identifier element fusion protein in the cell (for example, a promoter, terminator, ribosome binding site, and/or other elements well-known in the art). The expression construct may include, for example, a target polypeptide CDS, an amber stop, a TEV site, and an encoded polypeptide identifier element or peptide barcode (see, for example, FIG. 1), and can be a component of a multi-gene construct.

In certain example embodiments, target polypeptides expressed by a cell, a population of cells, or an acellular system in an individual discrete volume as disclosed herein are labeled with an origin specific nucleic acid identifier (“origin specific barcode”). Labeling may be through direct or indirect conjugation of the origin specific barcode to the target polypeptide. Direct conjugation of the origin specific barcode may be achieved by conjugation of the origin specific barcode to an amino acid side chain of the target polypeptide, for example a cysteine side chain. In certain example embodiments, the target polypeptide may be encoded to include a C-terminal cysteine to which the origin specific oligonucleotide may be conjugated. In certain other example embodiments, the target polypeptide may comprise one or more non-naturally (“un-natural”) occurring amino acids. Systems for producing polypeptides comprising non-naturally occurring amino acids are known in the art. See, e.g. Wang et al. Nature Chemistry. 2014, 6:393-403. Labeling of the origin specific barcode may then be achieve by conjugation of the origin specific barcode to an appropriate side chain of the non-naturally occurring amino acid. Alternatively, the incorporation of non-naturally occurring amino acids may then enable the conjugation of the origin specific barcode to the target polypeptide through a standard click chemistry reaction. For example the non-naturally occurring amino acids may contain ketones, azides, alkynes, alkenes, and tetrazine side chains genetically encoded in the polypeptide identifier element to enable site specific conjugation of a corresponding origin-specific nucleic acid identifier using for example a Cu(1)-catalyzed and strain-promoted Huisgen 1,3-dipolar cycloaddition (“Click reaction”). Labeling of target polypeptides with origin specific nucleic acids may also be achieved by indirect labeling. For example, the origin specific oligonucleotide may be attached to a first member of a binding pair that recognizes a corresponding binding partner attached to the target polypeptide, such as streptavidin and biotin. Alternatively, the origin specific barcode may incorporate one or more biotinylated nucleic acids which can then bind to streptavidin labeled target polypeptides. In certain example embodiments, the origin specific barcode is first conjugated to an antibody. The antibody may be specific to the target polypeptide of interest, or may recognize a common epitope on all of the expressed target polypeptides. For example, all target polypeptides may be encoded to include a common affinity tag or other epitope on a N- or C-terminus of the expressed polypeptide that is recognized by a common antibody or other generic protein binding molecule, such as Protein G, to which the various unique origin specific barcodes are attached. After expression and labeling of the target polypeptides with origin specific barcodes, all of the samples in the individual discrete volumes may be pooled and separated by an appropriate separation means, such as HPLC. Because the origin specific barcode retains information on the origin of each target polypeptide, a single pooled sample may be analyzed avoiding the need to do a separate analytical run on every single individual discrete volume. This allows for a highly multiplexed analysis. For example, hundreds of multiwell plates, or millions of droplets can be analyzed from a single HPLC run. After separation into polypeptide specific fractions, the origin specific barcodes can be removed from the target polypeptides and the sequence of the origin specific barcodes detected. All target polypeptides expressed in the same individual discrete volume will have been labeled with the same origin specific barcode and in this way target polypeptides expressed in the same individual discrete volume may then be identified.

In certain other example embodiments, target polypeptides include polypeptide identifier elements. For, example the one or more cells comprise one or more recombinant or synthetic constructs that encode the target polypeptide with a polypeptide identifier element such that the expressed target polypeptide includes the polypeptide identifier element.

In certain example embodiments, the polypeptide identifier element comprises a variable peptide sequence portion (“peptide barcode”) that has at least one physically separable property. Thus, each target polypeptide is labeled with a unique polypeptide sequence that is separable based on physical properties such as amino acid sequence, sequence length, charge, size, molecular weight, or hydrophobicity. Once expressed the polypeptide identifier element may then be labeled with an origin-specific barcode using any of the direct or indirect conjugation methods disclosed herein, including those discussed above in paragraph 163. The labeled polypeptide identifier element may then be separated from the target polypeptide and pooled into a single sample. Identification of each target polypeptide in the pooled sample may then be achieved by separating each peptide barcode based on the at least one physically separable property. Once identified, the origin specific barcodes for each peptide barcode specific fraction may be removed and pooled and the sequence of the origin specific barcodes in the pooled sample detected. Co-expressed target polypeptides may then be identified based on common origin specific barcodes as before.

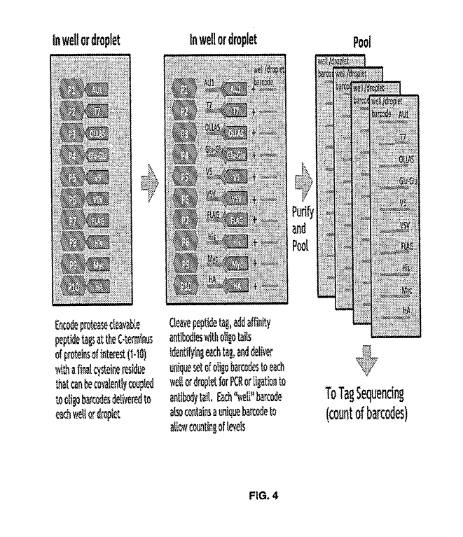

In certain other example embodiments, the polypeptide identifier element may comprise an affinity tag, with each target polypeptide assigned a specific corresponding affinity tag. After expression of the target polypeptide labeled with the polypeptide identifier, a set of protein binding molecules capable of binding to one of the affinity tags may be introduced into the individual discrete volumes. Each protein binding molecule may be previously labeled with an affinity tag specific nucleic acid identifier. The affinity tag specific nucleic acid identifier comprises a unique nucleic acid sequence that is specifically assigned to a corresponding affinity tag. An origin specific nucleic acid identifier specific for each individual discrete volume may also be introduced and appended, for example using a ligation reaction, to the affinity tag specific nucleic acid identifier connected to the protein binding molecule. These labeled polypeptide identifier complexes may then be removed from the target polypeptides and pooled. In certain example embodiments, the pooled target polypeptide elements are enriched by removing any unbound protein binding molecules. In certain example embodiments, the polypeptide identifier elements may further encode a common enrichment tag or epitope allowing the labeled polypeptide identifier element complexes to be separated from unbound protein binding molecules via the enrichment tag or epitope. The combined affinity tag specific and origin specific nucleic acid identifier may then be removed from the protein binding molecules bound to the polypeptide identifier element. The sequence of each combined nucleic acid identifier is then detected whereby the affinity tag specific nucleic acid identifier identifies a corresponding target polypeptide and the origin specific nucleic acid identifier identifies the individual discrete volume in which each target polypeptide was expressed. Target polypeptides expressed in the same individual discrete volume may the be grouped according to common origin specific nucleic acid identifier. See FIG. 4.

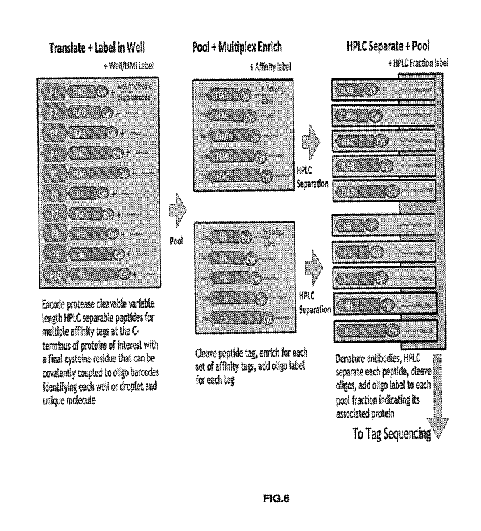

In certain example embodiments, the polypeptide identifier element comprises a set of constructs encoding target polypeptides with an affinity tag portion and a variable peptide sequence portion. This allows for multiple levels of multiplexing with the first level of multiplexing achieved using a set of affinity tags. Therefore a first subset of target polypeptides will have a polypeptide identifier element comprising a common first affinity tag, a second subset of target polypeptides will have a polypeptide identifier element with a second affinity tag and so forth for all affinity tags used. As before, after expression of the target polypeptides labeled with corresponding polypeptide identifiers is achieved, an origin specific nucleic acid identifier may be conjugated to the variable peptide sequence portion of the polypeptide identifier elements using any of the conjugation methods described herein, including those discussed in paragraph 163. The labeled polypeptide identifier element complexes may then be pooled and enriched into affinity tag specific fractions, for example, using affinity columns with antibodies to the corresponding affinity tags. Thus, a first fraction will have all labeled polypeptide identifier element complexes having a first affinity tag, a second fraction will have all labeled polypeptide identifier element complexes having a second affinity tag and so on for all affinity tags used. For each affinity tag specific fraction, an affinity tag specific nucleic acid identifier may be introduced and appended, for example by ligation reaction, to the existing origin specific nucleic acid identifiers. Each affinity tag specific identifier may comprise an unique nucleic acid sequence that is assigned to each affinity tag type. Each affinity tag specific fraction may then be resolved into polypeptide identifier element specific fractions based on a physical separable property of the labeled polypeptide identifier complex. For example, all polypeptide identifier elements having the same affinity tag will have different physical properties based at least in part on the variable peptide sequence portion. Accordingly, each common affinity tag should be paired with a unique variable peptide sequence portion. Each labeled polypeptide identifier element may then be separated into polypeptide identifier element specific fractions as discussed previously. For each polypeptide identifier element specific fraction, a fraction specific nucleic acid identifier may be introduced and appended to the existing origin specific and affinity tag specific nucleic acid identifiers. The combined nucleic acid identifier may then be removed from the polypeptide identifier element and pooled. The sequence of all combined nucleic acid identifiers is then determined, wherein the sequence of the combined affinity tag specific and fraction specific nucleic acid identifiers identify a corresponding target polypeptide and the origin specific nucleic acid identifier identifies the individual discrete volume in which a particular target polypeptide sequence was expressed. See FIG. 6.

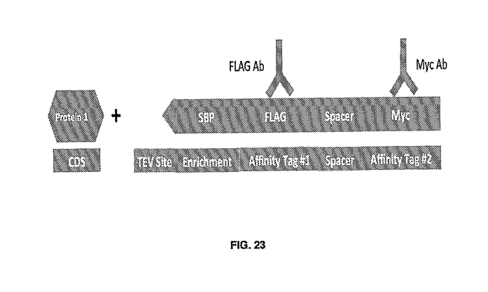

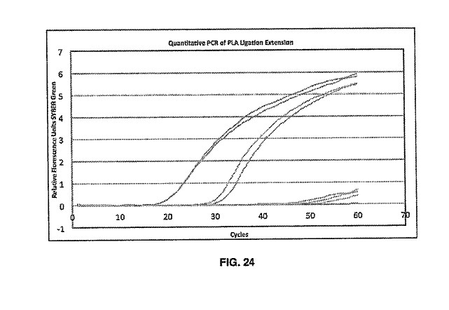

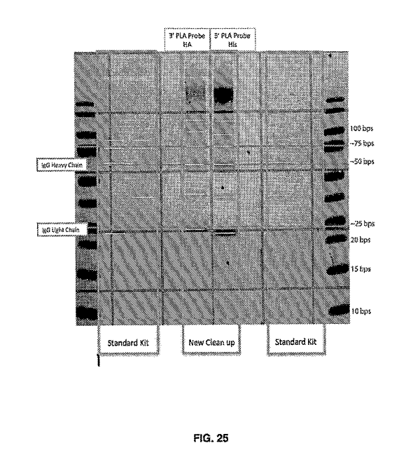

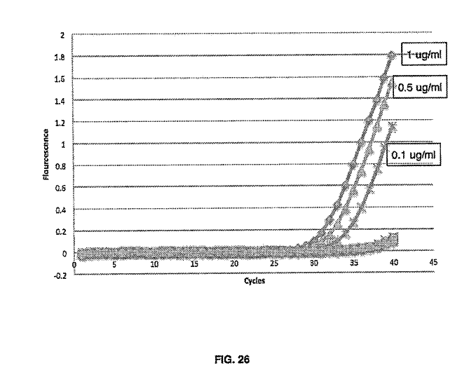

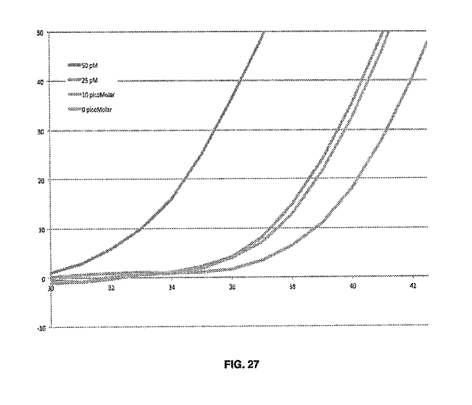

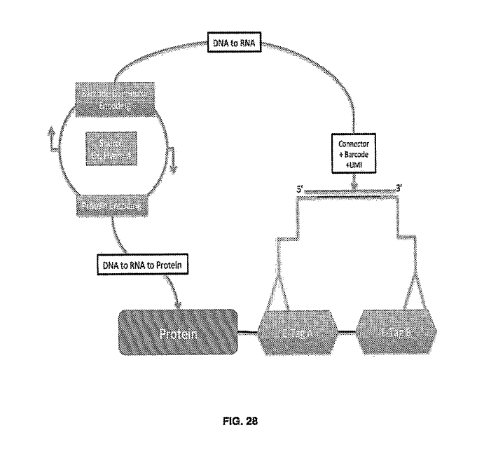

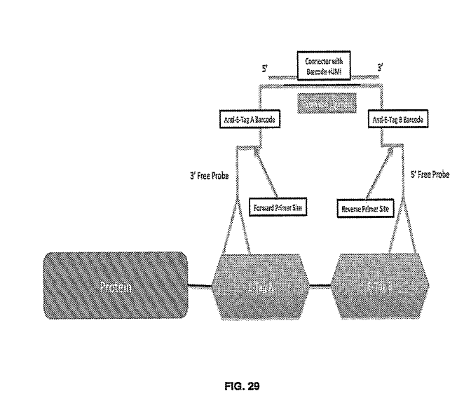

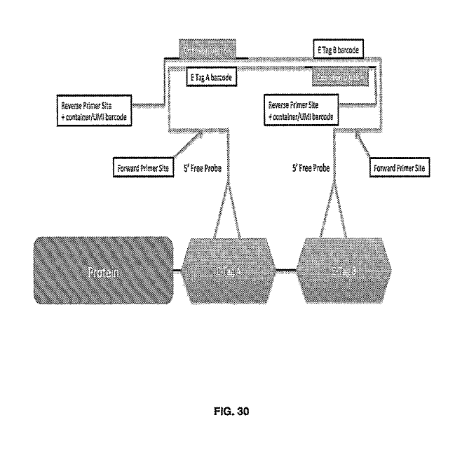

In certain example embodiments, the polypeptide identifier element comprises a combination of affinity tags. For example, a set of 14 affinity tags may be paired in various combinations to produce up to 49 unique polypeptide identifier elements. As before, constructs encoding target polypeptides are each assigned a unique polypeptide identifier element. After expression of the target polypeptide labeled with the polypeptide identifier is achieved, a set of protein binding molecules, such as antibodies may be introduced. Each protein binding molecule is labeled with affinity tag specific nucleic acid identifier (“proximity probe”). The proximity probes may have a free 5′ or a free 3′ end. In certain embodiments both probes will have 5′ free ends, both probes have 3′ free ends, or one probe has a free 5′ end and the other probe has a free 3′ end. The set of protein binding molecules is introduced into each individual discrete volume under conditions sufficient to allow binding of the protein binding molecules to each protein binding molecules' corresponding affinity tag. Accordingly, for each polypeptide identifier element the corresponding protein binding molecules will bind proximate to one another on the polypeptide identifier element. A set of connector nucleic acids may then be introduced into each individual discrete volume. Each connector nucleic acid comprises a sequence that can hybridize with at least a portion of an affinity tag specific identifier or a specific pair of affinity tag specific identifiers on the protein binding molecules. The connector nucleic acid further comprises a origin specific nucleic acid identifier sequence in between the affinity tag identifier binding regions, which may be located 5′ and 3 of the origin specific nucleic acid identifier. In certain example embodiments, a set of connector nucleic acids is introduced into each individual discrete volume with each connector comprising the same origin specific nucleic acid identifier portion and a combination of the affinity tag binding portions capable of binding one of the possible affinity tag pairs. The polypeptide identifier element may be removed from the target polypeptide before introduction of the protein binding molecules or prior to introduction of the connector nucleic acid. In certain example embodiments, the polypeptide identifier further encodes a protease recognition site to facilitate removal of the polypeptide identifier from the target polypeptide. After the connector nucleic acid binds to the corresponding affinity tag identifiers on the protein binding molecules, the connector nucleic acid sequence is used to facilitate a ligation extension reaction. The resulting ligation extension product comprises the affinity tag nucleic acid identifier information and origin specific identifier nucleic acid information. The extension product may then be isolated, amplified as needed, and the sequence of the extension product detected wherein the affinity tag identifier sequences identify the corresponding polypeptide identifier, which identifies a corresponding target polypeptide, and the origin specific nucleic acid identifier identifies the individual discrete volume in which that nucleic acid identifier was expressed. See FIGS. 22, 23, 28, and 29.

In certain example embodiments, the affinity tag identifiers on the protein binding molecules comprise a first binding region to which a connector nucleic acid may bind through base pairing interactions, an extension barcode section identifying the protein binding molecule, and a second binding region that can bind to a neighboring affinity tag identifier through base pair interactions. The affinity tag identifier may have a 5′ free end. The affinity tag identify may further comprise a forward primer site. The corresponding connector molecule may comprising a binding region that recognizes and binds to the corresponding first binding region on the affinity tag identifier, and an origin specific nucleic acid identifier. Upon binding of the protein binding molecules to their respective affinity tag portions of the polypeptide identifier element, the affinity tag identifiers bind to one another through the corresponding second binding regions, for example through hybridization. The connector nucleic acids are then introduced into the individual discrete volumes. The connector nucleic acids bind to the first binding region on one affinity tag identifier and proximate to the free end of the neighboring affinity tag identifier such that the intervening sequence between the end of an affinity tag identifier and the connector nucleic acid is bridged by the extension barcode section of the affinity tag identifier to which the connector nucleic acid is bound. See FIG. 30. The extension portion than facilitate an extension ligation that incorporates the affinity tag specific barcodes and the origin specific barcodes of the connector nucleic acids into an extension ligation product. The extension ligation product may then be amplified and detected.

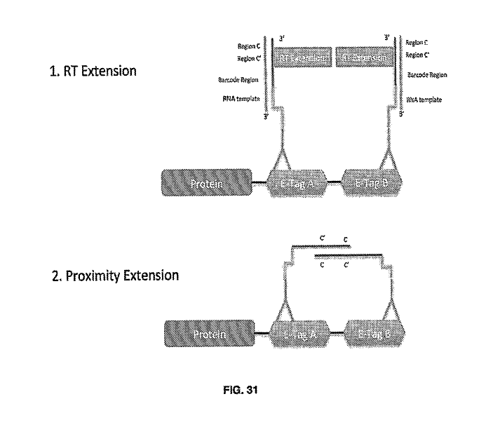

In another example embodiment, the affinity tag identifiers comprise a first binding region that hybridizes to a corresponding binding region on the connector nucleic acids. The connector nucleic acid further encode an origin specific nucleic acid sequence and a complementarity portion. The connector nucleic acids bind to the affinity tag via the first binding region, for example through hybridization. Reagents sufficient to conduct a reverse transcriptase reaction are then introduce wherein the connector nucleic acid serves as a template to extend a 5′ free end of the affinity tag identifier. See FIG. 31. The RT reaction incorporates the origin specific nucleic acid sequence and the complementarity region of the connector nucleic acid template into the extended portion of the affinity tag identifier. The proximate affinity tag identifiers may then hybridize to one another via the complementarity regions. The free ends of each affinity tag identifier may then be extended further as needed. The resulting extension product therefore incorporates the origin specific nucleic acid sequence and may be amplified and detected to identify the sequence of the origin specific nucleic acids and the affinity tag identifiers.

In certain example embodiments, the constructs encoding the target polypeptide may further encode the connector nucleic acids. When the constructs are created a self-identifying nucleic acid sequence transcript is designed into the connector molecule. The connector molecule may be under the control of an inducible or constitutively active promoter. The self-identifying sequence may be designed or semi-randomly encoded to uniquely identify each construct. A library of such constructs may be prepared so that each individual discrete volume receives a construct encoding a unique self-identifying sequence. In this way the identity of the individual discrete volume and origin of target molecules expressed therein may be tracked.

In certain example embodiments, the polypeptide identifier may further comprise one or more spacers between the affinity tags to reduce steric hindrance between two protein binding molecules binding to each affinity tag. The spacer may be 10 to 20 amino acids long. In certain example embodiments, the spacer is glycine rich. In certain example embodiments, the polypeptide identifier may further comprises a common enrichment epitope, allowing labeled polypeptide identifier elements to be separated from unbound protein binding molecules.

In certain example embodiments, the connector nucleic acid comprise affinity tag nucleic acid identifier binding regions. For example, the connector nucleic acid may have a first affinity tag nucleic acid identifier binding region capable of hybridizing to at least a portion of a first affinity tag nucleic acid identifier on a 5′ end of the connector molecule, and a second affinity tag nucleic acid identifier binding region capable of hybridizing to at least a portion of a second affinity tag nucleic acid identifier on the 3′ end. An origin specific nucleic acid identifier is located between the two affinity tag nucleic acid binding regions. Accordingly, the connector nucleic acid is able to bridge the gap and connect two proximate affinity tag nucleic acid identifiers on two corresponding protein binding molecules bound to their respective affinity tags in the polypeptide identifier elements. See FIG. 23. The connector nucleic acid is used to facilitate an extension ligation reaction resulting in a ligation product that comprises the affinity tag identifiers from each protein binding molecule and the origin specific nucleic acid sequence from the connector oligonucleotide. The sequence may be amplified as needed and the sequence detected, whereby the affinity tag nucleic acid identifiers identify the polypeptide identifier element and thus the corresponding target polypeptide, and the origin specific nucleic acid identifier identifies the individual discrete volume in which the target polypeptide was expressed.

In certain example embodiments, the connector oligonucleotide comprise a affinity tag binding region that can hybridize to at least a portion of an affinity tag nucleic acid identifier on a protein binding molecule, a origin specific nucleic acid identifier a universal complementarity region. The connector oligonucleotide binds to a corresponding affinity tag nucleic acid identifier and is then used as a template for a reverse transcription reaction that extends the affinity tag identifier to further incorporate the origin specific nucleic acid identifier and complementarity regions encoded in the connector nucleic acid. See FIG. 24. The complementarity regions now included in the affinity nucleic acid identifiers allow to proximate affinity nucleic acid identifiers to hybridize together via the complementarity regions. In certain example embodiments, it may be necessary to fill any gaps with a second extension reaction because of the additional nucleic acids introduced by the reverse transcription step and/or to ensure the affinity tag nucleic acid identifiers are incorporated in the extension product. The extension product may be amplified as needed and the sequence of the extension product detected, where the affinity tag nucleic acid identifiers identify the polypeptide identifier element and thus the corresponding target polypeptide, and the origin specific nucleic acid identifier identifies the individual discrete volume in which the target polypeptide was expressed.

A target molecule may also be a nucleic acid, such as a DNA or RNA molecule. For example, an RNA molecule transcribed from a gene of interest (or a corresponding cDNA molecule) can be labeled according to the methods of the disclosure and subsequently isolated and sequenced with its barcode label. A plurality of such labeled nucleic acids can be simultaneously labeled, for example, with each nucleic acid receiving a distinct barcode and/or one or more unique molecular identifiers and barcode receiving adapters. In certain embodiments, the nucleic acid is produced by a cell, cell lysate, or cell-free extract. In particular embodiments, the nucleic acid is produced by a microbe, such as a prokaryotic cell. In some embodiments, a nucleic acid target molecule encodes a polypeptide target molecule in the same discreet volume.

In some embodiments, a target molecule may be associated with diseased cells or a disease state. For instance, a target molecule may be associated with cancer cells, for example, a protein, polypeptide, or nucleic acid selectively expressed or not expressed by cancer cells, or may specifically bind to such a protein or polypeptide (for example, an antibody or fragment thereof, for example, as described herein). In certain instances, the target molecule is a tumor marker, for example, a substance produced by a tumor or produced by a non-cancer cell (for example, a stromal cell) in response to the presence of a tumor. Many tumor markers are not exclusively expressed by cancer cells, but may be expressed at altered (i.e., elevated or decreased) levels in cancerous cells or expressed at altered (i.e., elevated or decreased) levels in non-cancer cells in response to the presence of a tumor. In some embodiments, the target molecule may be a protein, polypeptide, or nucleic acid expressed in connection with any disease or condition known in the art.

Target molecules (for example, proteins and nucleic acid molecules) can optionally be expressed in cells or in extracts, such as cell free extracts, which optionally are present in discrete volumes as described herein. In various examples, a target molecule (for example, a polypeptide or a carbohydrate) is present on the surface of a cell (for example, an antibody on a B-cell or a cell surface receptor). Thus, in some embodiments, a target molecule, such as a protein or polypeptide, is one that is normally found on the surface of a cell. In other embodiments, a target molecule, such as a protein or polypeptide, is not normally found on the surface of a cell. In certain instances, such a target molecule is normally found within a cell, such as in the cytoplasm or in an organelle. In these examples, it may be necessary to lyse a cell in which the target molecule is produced in order to label it or an associated tag. Protein or nucleic acid target molecules can be naturally produced by a cell or can be recombinantly produced based on, for example, the presence of a synthetic construct in a cell.

The discrete volumes or spaces, as disclosed herein mean any sort of area or volume which can be defined as one where the barcoded molecules, such as labeled target molecules or labeled nucleic acids are not free to escape or move between. Discrete volumes include droplets, such as the droplets from a water-in-oil emulsion, or as deposited on a surface, such as a microfluidic droplet, for example deposited on a slide. Other types of discrete volumes include with out limitation a tube, well, plate, pipette, pipette tip, and bottle. Other types of discrete volumes include “virtual” containers, such as defined by areas exposed to light, diffusion limits, or electro-magnetic means. Such discrete volumes can also exist by diffusion defined volumes, or spaces that are only accessible to certain molecules or reactions because diffusion constraints effectively defining a space, for example, chemically defined volumes or spaces where only certain target molecules can exist because of their chemical or molecular properties such as size, or electro-magnetically defined volumes or spaces where the electro-magnetic properties of the target molecules or their supports such as charge or magnetic properties can be used to define certain regions in a space. Such discrete may also be optically defined volumes or spaces that may be defined by illuminating it with visible, ultraviolet, infrared, or other wavelengths of light such that only target molecules within the defined space may be labeled. Such discrete volumes can be composed of, for example, plastic, metal, composite materials, and/or glass. Such discrete volumes can be adapted for placement into a centrifuge (for example, a microcentrifuge, an ultracentrifuge, a benchtop centrifuge, a refrigerated centrifuge, or a clinical centrifuge). A discreet volume can exist on its own, as a separate entity, or be part of an array of such discreet volumes, for example, in the form of a strip, a microwell plate, or a microtiter plate. A discrete volume can have a capacity of, for example, at least about 1 femtoliter (fl) to about 1000 ml, such as about 1 fl, 10 fl, 100 fl, 250 fl, 500 fl, 750 fl, 1 picoliter (pl), 10 pl, 100 pl, 250 pl, 500 pl, 750 pl, 1 nl, 10 nl, 100 nl, 250 nl, 500 nl, 750 nl, 1 μl, 5 μl, 10 μl, 20 μl, 25 μl, 50 μl, 100 μl, 200 μl, 250 μl, 500 μl, 750 μl, 1.25 ml, 1.5 ml, 2 ml, 2.5 ml, 5 ml, 10 ml, 15 ml, 20 ml, 25 ml, 50 ml, 100 ml, 150 ml, 200 ml, 250 ml, 300 ml, 350 ml, 400 ml, 450 ml, 500 ml, 550 ml, 600 ml, 650 ml, 700 ml, 750 ml, 800 ml, 900 ml, or 1000 ml.

In certain example embodiments, a discrete volume is a droplet, such as a droplet in an emulsion and/or a microfluidic droplet. Emulsification can be used in the methods of the disclosure to separate or segregate a sample or set of samples into a series of discrete volumes, for example a discrete volume having a singe cell or a discrete portion of an acellular sample, such as a cell-free extract or a cell-free transcription and/or cell-free translation mixture. Typically, as used in conjunction with the methods and compositions disclosed herein, an emulsion will include a plurality of droplets, each droplet including one or more target molecules and/or target nucleic acids and an origin-specific barcode, such that each droplet includes a unique barcode that distinguishes it from the other droplets. Emulsification can be used in the methods of the disclosure to discrete volumealize one or more target molecules in emulsion droplets with one or more nucleic acid barcodes, such as origin specific barcodes. An emulsion, as disclosed herein, will typically include a plurality of droplets, each droplet including one or more target molecules, target nucleic acids and one or more nucleic acid barcodes, such as origin specific barcodes. Droplets in an emulsion can be sorted and/or isolated according to methods well known in the art. For example, double emulsion droplets containing a fluorescence signal can be analyzed and/or sorted using conventional fluorescence-activated cell sorting (FACS) machines at rates of >10⁴ droplets s⁻¹, and have been used to improve the activity of enzymes produced by single cells or by in vitro translation of single genes (Aharoni et al., Chem Biol 12(12):1281-1289, 2005; Mastrobattista et al., Chem Biol 2(12):1291-1300, 2005). However, the emulsions are highly polydisperse, limiting quantitative analysis, and it is difficult to add new reagents to pre-formed droplets (Griffiths et al., Trends Biotechnol 24(9):395-402, 2006). These limitations can, however, be overcome by using protocols based on droplet-based microfluidic systems (see for example Teh et al., Lab on a chip 8(2):198-220, 2008; Theberge et al., Angew Chem Int Ed Engl 49(34):5846-5868, 2010; and Guo et al., Lab on a chip 12(12):2146, 2012) in which highly monodisperse droplets of picoliter volume can be made (Anna et al., Appl Phys Lett 82(3):364-366, 2003), fused (Song et al., Angew Chem Int Edit 42(7):767-772, 2003; Chabert et al., Electrophoresis 26(19):3706-3715, 2005), split (Song et al., Angew Chem Int Edit 42(7):767-772, 2003; Link et al., Phys Rev Lett 92(5):054503, 2004), incubated (Song et al., Angew Chem Int Edit 42(7):767-772, 2003; Frenz et al., Lab on a chip 9(10):1344-1348, 2009), and sorted triggered on fluorescence (Baret, et al., Lab on a chip 9(13):1850-1858, 2009), at kHz frequencies, such as those described in Mazutis et al. (Nat. Protoc. 8(5): 870-891, 2013), incorporated by reference herein. As disclosed herein, an emulsion can include various compounds, enzymes, or reagents in addition to the target molecules, target nucleic acids and origin-specific barcodes. These additives may be included in the emulsion solution prior to emulsification. Alternatively, the additives may be added to individual droplets after emulsification.

Emulsion may be achieved by a variety of methods known in the art (see, for example, US 2006/0078888 A1, of which paragraphs [0139]-[0143] are incorporated by reference herein). In some embodiments, the emulsion is stable to a denaturing temperature, for example, to 95° C. or higher. An exemplary emulsion is a water-in-oil emulsion. In some embodiments, the continuous phase of the emulsion includes a fluorinated oil. An emulsion can contain a surfactant or emulsifier (for example, a detergent, anionic surfactant, cationic surfactant, or amphoteric surfactant) to stabilize the emulsion. Other oil/surfactant mixtures, for example, silicone oils, may also be utilized in particular embodiments. An emulsion can be contained in a well or a plurality of wells, such as a plate, for easy of handling. In some examples, one or more target molecules, target nucleic acid and nucleic acid barcodes are discrete volumealized. An emulsion can be a monodisperse emulsion or a polydisperse emulsion. Each droplet in the emulsion may contain, or contain on average, 0-1,000 or more target molecules. For instances, a given emulsion droplet may contain 0, 10, 20, 30, 40, 50, 100, 200, 300, 400, 500 or more target molecules. In particular embodiments, a given droplet may contain 0, 1, 2, or 3 cells capable of expressing or secreting target molecules, for example, a clonal population of target molecules. On average, the droplets of an emulsion of the present in disclosure may contain 0-3 cells capable of expressing or secreting target molecules, such as 0, 1, 2, or 3 cells capable of expressing or secreting target molecules, as rounded to the nearest whole number. In some embodiments, the number of cells capable of expressing or secreting target molecules in each emulsion droplet, on average, will be 1, between 0 and 1, or between 1 and 2. In other embodiments, the droplet may contain an acellular system, such as a cell-free extract.

Compartmentalization of target molecules, target nucleic acids and nucleic acid barcodes into wells can be achieved, in some embodiments, due to physical limitations relating to the mass or dimensions of the target molecules and nucleic acid barcodes, the dimensions of the well, or a combination thereof. A well may be a fiber-optic faceplate where the central core is etched with an acid, such as an acid to which the core-cladding is resistant. A well may be a molded well. The wells may be covered to prevent communication between the wells, such that the beads present in a particular well remain within the well or are inhibited from moving into a different well. The cover may be a solid sheet or physical barrier, such as a neoprene gasket, or a liquid barrier, such as fluorinated oil. Methods applicable to the present disclosure are known in the art (for example, Shukla et al., J. Drug Targeting 13: 7-18, 2005; Koster et al., Lab on a Chip 8: 1110-1115, 2008).

In certain embodiments, the single cells or a portion of the acellular system from the sample are encapsulated together with a bead, such as a hydrogel bead that includes the origin-specific barcodes reversibly coupled thereto. A set of hydrogel beads, such as PEG-DA beads, of uniform size is created, for example, using a PDMS chip. In some embodiments, the uniformly sized PEG-DA hydrogel bead are co-polymerized with a generic capture oligonucleotide, which can be used to build a nucleic acid identification sequence unique to each bead. Using automation techniques and split-pool labeling (see for example International Patent Publication No. WO2014/047561, which is specifically incorporated by reference) a unique nucleic acid barcode can be added to each bead. Using microfluidics, the individual beads can be placed into single drop and then single cells added, such that each drop in the emulsion contains a single cell and single hydrogel containing a unique origin-specific bar code. As shown in the FIG. 1, this system can be used to label all of the amplicons derived from a cell with a unique barcode. If the emulsion is then broken, the result is a pooled sample of amplicons barcoded according to droplet. This all of the amplicons can be traced back to the singe cell from which they originated. In some embodiments, a bead includes an exemplary bead and origin-specific barcode for labeling a target nucleic acid. In specific embodiments, the origin-specific barcodes are delivered to the discrete volumes by delivering a single bead to each discrete volume wherein each bead carries multiple copies of a single origin-specific barcode sequence.

In some embodiments, the methods further include amplifying one or more of the origin-specific barcodes, the target molecule specific binding agent barcodes, test-agent specific barcodes, the target nucleic acid barcodes, and the target molecule barcodes.

In some embodiments, the methods further include detecting one or more of the target molecule specific binding agent barcodes, test-agent specific barcodes, the target nucleic acid barcodes, and the target molecule barcodes, for example detecting the sequence of the origin-specific barcodes, the target molecule specific binding agent barcodes, test-agent specific barcodes, the target nucleic acid barcodes, and/or the target molecule barcodes with hybridization, sequencing, or a combination thereof.

In some embodiments, the methods further include quantifying or more of the origin-specific barcodes, the target molecule specific binding agent barcodes, test-agent specific barcodes, the target nucleic acid barcodes, and the target molecule barcodes. In some embodiment labeled target molecules to a reaction condition and attaching a condition-specific barcode to each of the sample-specific barcodes labeling the isolated labeled target molecules, thereby forming a conjugate barcode.

In some embodiments, the method further includes attaching further condition-specific barcodes to the origin-specific barcodes, wherein each distinct condition-specific barcode is associated with a distinct condition, such as exposure to a particular temperature, pH, small molecule, nucleic acid, peptide, carbohydrate, lipid, solute concentration, solvent, or filter.

In some embodiments, one or more of the discrete volumes include a reporter cell expressing one or more mRNAs; where the quantity of one or more of the mRNAs expressed by the reporter cell within a discrete volume varies in response to the presence, amount, or activity of a target molecule in the discrete volume and wherein the mRNA or corresponding cDNA in a discrete volume is labeled with the same origin-specific nucleic acid barcode as the target molecule in the discrete volume.

In some embodiments, linking the polypeptides of interest to the peptide identifier elements, includes inserting the nucleic acid sequence encoding peptide identifier elements into the genome of a cell, wherein the nucleic acid sequence encoding the polypeptides of interest to the nucleic acid sequence encoding peptide identifier elements are operatively linked. In certain embodiments, inserting the nucleic acid sequence encoding peptide identifier elements includes using a genome editing system, such as a CRISPR, system, a TALEN system, a ZFN system, a meganuclease and the like.

As disclosed herein, mutations in cells, such as the insertion of peptide identifier elements, can be made by way of the CRISPR-Cas system or a Cas9-expressing eukaryotic cell or a Cas-9 expressing eukaryote. The Cas9-expressing eukaryotic cell or eukaryote, can have guide RNA delivered or administered thereto, whereby the RNA targets a loci and induces a desired mutation for use in or as to the invention. With respect to general information on CRISPR-Cas Systems, components thereof, and delivery of such components, including methods, materials, delivery vehicles, vectors, particles, and making and using thereof, including as to amounts and formulations, as well as Cas9-expressing eukaryotic cells, Cas-9 expressing eukaryotes, such as a mouse, reference is made to: U.S. Pat. Nos. 8,697,359, 8,771,945, 8,795,965, 8,865,406, 8,871,445, 8,889,356, 8,889,418, 8,895,308, 8,932,814, 8,945,839, 8,906,616; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents/Patent Applications: EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), WO2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), andv Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013); RNA-guided editing of bacterial genomes using CRISPR-Cas systems. Jiang W., Bikard D., Cox D., Zhang F, Marraffini L A. Nat Biotechnol March; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500(7463):472-6. doi: 10.1038/Nature12466. Epub 2013 Aug. 23; Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5. (2013); DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013); Genome engineering using the CRISPR-Cas9 system. Ran, F A., Hsu, P D., Wright, J., Agarwala, V., Scott, D A., Zhang, F. Nature Protocols November; 8(11): 2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27. (2014). 156(5):935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) Apr. 20. doi: 10.1038/nbt. 2889; CRISPR-Cas9 Knockin Mice for Genome Editing and Cancer Modeling, Platt et al., Cell 159(2): 440-455 (2014) DOI: 10.1016/j.ce11.2014.09.014; Development and Applications of CRISP R-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 January 3; 343(6166): 80-84. doi:10.1126/science.1246981; Rational design of highly active sgRNAs for CRISPR-Cas9-mediated gene inactivation, Doench et al., Nature Biotechnology 32(12):1262-7 (2014) published online 3 Sep. 2014; doi:10.1038/nbt.3026, and In vivo interrogation of gene function in the mammalian brain using CRISPR-Cas9, Swiech et al, Nature Biotechnology 33, 102-106 (2015) published online 19 Oct. 2014; doi:10.1038/nbt.3055, each of which is incorporated herein by reference.

As disclosed herein, mutations in cells, such as the insertion of peptide identifier elements, can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

As disclosed herein, mutations in cells, such as the insertion of peptide identifier elements, can be made by way of the zinc-finger nucleases (ZFNs) system. The ZFN system uses artificial restriction enzymes generated by fusing a zinc finger DNA-binding domain to a DNA-cleavage domain that can be engineered to target desired DNA sequences. xemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. As disclosed herein, mutations in cells, such as the insertion of peptide identifier elements, can be made by way of meganucleases, which are endodeoxyribonucleases characterized by a large recognition site (double-stranded DNA sequences of 12 to 40 base pairs). Exemplary method for using megonucleases can be found in U.S. Pat. Nos. 8,163,514; 8,133,697; 8,021,867; 8,119,361; 8,119,381; 8,124,369; and 8,129,134, which are specifically incorporated by reference.

### C. Example Embodiments

In one example embodiment, a method for multiplex analysis of polypeptides in samples comprising providing a sample comprising one or more cells or an acellular system. All or a portion of the sample is distributed into individual discrete volumes as described above. The samples are then cultured in conditions sufficient to allow for expression of polypeptides and nucleic acids, such as but not limited to mRNA. The target polypeptides and nucleic acids may be native or may be introduced by one or more recombinant or synthetic constructs. The expressed polypeptides and/or nucleic acids are then labeled by introducing an origin-specific nucleic acid identifier (“origin specific barcode”) into each individual discrete volume. The origin specific nucleic acid identifier introduced into each individual discrete volume is unique to that individual discrete volume. That is the origin specific nucleic acid identifier comprises a unique nucleotide sequence that corresponds to each individual discrete volume. The origin specific nucleic acid identifier may be introduced by any of the methods disclosed above. Labeling

### D. Exemplary Applications

1. Proteomics

The methods of the disclosure can be used in proteomics applications in order to, for example, assess expression levels and/or functions of various proteins expressed in cells or in acellular systems. The proteins can optionally be encoded by synthetic constructs (for example, multi-gene constructs), or can be proteins naturally expressed in wild-type cells.

In various examples, the proteins are components of a metabolic pathway, and the proteomic analysis can be carried out, for example, to assemble and optimize novel pathways and/or to identify rate limiting steps. With respect to the latter, based on the results of the analysis, expression of a rate limiting component of a pathway can be altered in order to optimize the pathway. These approaches can be used, for example, in the context of optimizing metabolically engineered microorganisms in synthetic biology.

In other applications, the proteomics methods of the disclosure can be used in the identification and verification of biomarkers for disease, such as cancer, and optionally can be used to assess proteome changes in cells exposed to different conditions. The different proteins analyzed using the methods of the disclosure can be different components of a pathway and/or sequence variants of a base sequence, in which case the methods can be used to identify variants having particular expression levels, stabilities, or other functional features. The variations assessed can range from individual amino acid substitutions to domain or subunit substitutions or swaps, and can include any number of combinations thereof. The variations can be random or can be generated by, for example, mixing and matching of sequences derived from, for example, different species.

The proteomics methods of the disclosure involve the use of polypeptide identifier elements, which can optionally be encoded within the same nucleic acid molecules as the target proteins that they are tagging (for example, at the amino or carboxyl terminal thereof). In general, target proteins are labeled with polypeptide identifier elements (for example, encoded polypeptide identifier elements), which can optionally include an affinity tag and/or a peptide barcode. Target proteins (or, more typically, associated polypeptide identifier elements) within a discrete volume (for example, a droplet or a well) are labeled with origin-specific nucleic acid barcodes to facilitate later identification of the discrete volume from which associated target proteins having desired features derive. After origin-specific nucleic acid barcoding of, for example, polypeptide identifier elements associated with target proteins (which optionally is accompanied by origin-specific nucleic acid barcoding of corresponding nucleic acid molecules), pooled polypeptide identifier elements (optionally cleaved from their respective target proteins) can be enriched for and/or fractionated based on various properties (for example, size, hydrophobicity, and/or binding specificity), and each specific separation step can be associated with the addition of a separation step-specific nucleic acid barcode. Optionally, the different nucleic acid barcodes added to, for example, a particular target polypeptide identifier element are linked together, leading to the formation of a unique nucleic acid barcode concatemer. Sequencing of the concatemer can be done to identify, characterize, and/or quantify the associated target protein, and will specifically provide information relating to source (i.e., discrete volume) and physical properties (depending on modes of separation used).

Details of various possible features of the proteomics methods of the disclosure are described below in connection with FIG. 1-3, while specific examples of the methods are set forth in FIG. 4-7. FIG. 1 is a diagram showing protein labeling via encoded polypeptide identifier elements. FIG. 1A shows a multi-gene construct and, for one of the genes, sequences 3′ to the coding sequence (CDS) and encoding a proteolytic cleavage site (TEV) and a peptide barcode are highlighted. Tag/peptide barcode combinations can be designed so as to cause minimal interference with target protein function, for example with the use of a peptide linker. FIG. 1B shows exemplary fusion proteins that can be encoded by a construct such as that illustrated in FIG. 1A. The fusion proteins include target proteins (arbitrarily assigned identifiers P1-P96) with C-terminal polypeptide identifier elements that each include, in this example, two information-carrying regions: a variable length peptide barcode and an affinity tag (T) (for example, FLAG, MYC, HA, etc.). The possible number of different polypeptide identifier elements (96, in this example) is based on the number of different combinations that can be made between the different peptide barcodes and affinity tags. An encoded protein, including a target protein and an encoded polypeptide identifier element, can also include a cleavage site between the target protein and the polypeptide identifier element so that the polypeptide identifier element can be cleaved from the target protein after origin-specific nucleic acid barcode labeling, as explained further below. As shown in FIG. 1B, the affinity tags can be bound by antibodies containing isotope labels, such as labels have a predictable mass difference, or cleavable nucleic acid barcodes, for sample multiplexing or massive multiplexing, respectively.

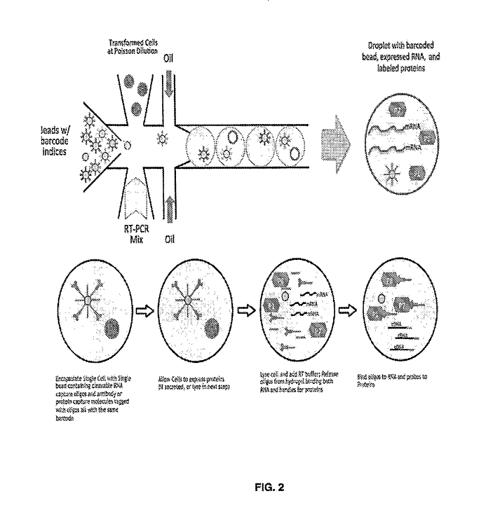

In certain examples of the proteomics methods of the disclosure, it is desirable to label both proteins produced by a cell (or in a cell free mixture), and also expressed nucleic acid molecules (for example, nucleic acid molecules encoding target proteins). FIG. 2 is a diagram showing a scheme for high-throughput proteomics combined with digital gene expression (DGE) in emulsion droplets that can be used to achieve this purpose. Individual cells are encapsulated in droplets with individual beads containing capture molecules, which include (i) cleavable antibody/protein capture molecules, and (ii) RNA/cDNA capture oligos, with each capture molecule or oligo being tagged with the same origin-specific nucleic acid barcode. The nucleic acid barcodes attached to an individual bead each include the same origin-specific sequence. Each encapsulated cell expresses its target proteins, which are exposed to antibody/protein capture molecules directly, if the target proteins are secreted, or are released from the cells by lysis. The antibody/protein capture molecules attach to their respective targets, thus associating the origin-specific nucleic acid barcodes with target proteins of interest. The RNA/cDNA capture oligos attach to RNA, or cDNA produced from RNA, which optionally encodes the target proteins. This process results in the labeling of both the target proteins and nucleic acids that, for example, encode the target proteins, with the same or matching origin-specific nucleic acid barcodes. An origin-specific barcode may contain, for example, a region of complementarity an mRNA encoding a target protein (for example, a poly-T region), and can then operate as an RT-PCR primer. As such, reverse transcription of the mRNA utilize the origin-specific barcode as a primer, thereby adding the origin-specific barcode to the resultant cDNA molecule. The labeled proteins can then be further processed (for example, by use of affinity and/or size separation steps). Determination of the origin-specific nucleic acid barcodes, and any other nucleic acid sequence can be carried out and then proteomic information can be associated with corresponding transcriptome information due to the common or matched origin-specific barcodes used to tag proteins and expressed nucleic acid molecules of the same discrete volume.

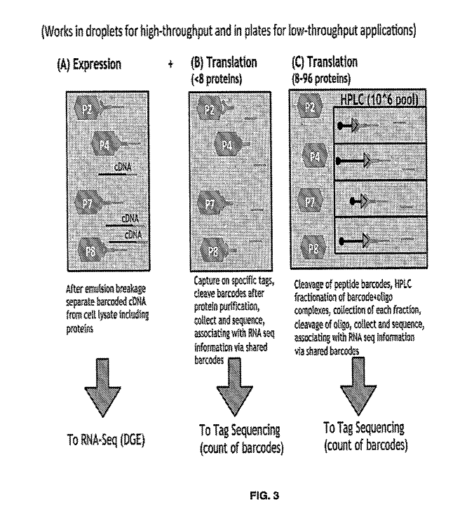

FIG. 3A further illustrates the labeling of proteins and expressed nucleic acid molecules within a discrete volume with common origin-specific nucleic acid barcodes, thus permitting correlation between target protein and mRNA expression. As shown in FIG. 3A, after emulsion breakage, barcode tagged cDNA is separated from a cell lysate for processing by RNA-Seq (DGE), while FIG. 3B shows nucleic acid barcode tagged proteins being captured based on the presence of specific affinity tags, after which nucleic acid barcodes are cleaved from the proteins and collected and the sequence determined. Information obtained from the protein-associated nucleic acid barcodes is then associated with RNA-Seq information via shared barcodes. FIG. 3B indicates the applicability of this method for 8 or fewer proteins, based on the use of 8 or fewer specific affinity tags. To achieve greater multiplexing, variable length peptide barcodes can be used, as shown in FIG. 3C, optionally in combination with multiple affinity tags. In this example, encoded polypeptide identifier elements, including variable length peptide barcodes, are cleaved from target proteins and fractionated (for example, by HPLC), after which nucleic acid barcodes are cleaved, collected, and sequenced for association with RNA-Seq information via shared barcodes.

The features of the proteomics methods of the disclosure are described further, below, in the context of specific examples of carrying out these methods (see Examples 1-3).

2 Rapid Prototyping in Cell-Free Mixes

A central facet of synthetic biology is the engineering of genetic constructs, for example, from libraries of genetic elements collectively referred to herein as “parts.” The disclosure provides methods for rapid prototyping and screening of such genetic constructs. Methods and kits for generating sequence-verified pools of such genetic constructs, as well as such pools, are described, for example, in U.S. Provisional Application No. 62/003,331, which is incorporated herein by reference in its entirety. In these methods, genetic designs are assembled in a high-throughput manner from modular parts, which is followed by their tagging with unique barcodes and sequence verification using, for example, next-generation sequencing technologies. Desired sequence-verified permutations can be subsequently retrieved by, for example, tag-directed PCR retrieval techniques.

The rapid prototyping methods of the disclosure can be used in connection with, for example, high-value organisms and organelles that may be difficult to transform and/or assay, such as non-model organisms (for example, Saccharopolyspora spinosa, Myxococcus, plant chloroplasts, or other organisms described herein). In particular, the present disclosure features cell-free prototyping synthesis (CFPS) systems in which a cell-free mix derived from, for example, a high-value organism or organelle, is encapsulated in a discrete volume (for example, an emulsion droplet) with one or more genetic constructs to be tested. The discrete volumes are then incubated under conditions permitting expression of one or more genes in the genetic construct(s), and the mRNA and/or gene products (for example, proteins, non-coding RNAs, peptides, or complexes thereof) can be tagged with barcodes according to the methods of the disclosure. The expressed parts can be assayed as desired, for example, prior to or after barcoding. A given gene product can be tagged with the same barcode as the mRNA molecule encoding it, thus allowing for genotype-phenotype coupling as described herein. Thus, barcodes can be sequenced and/or combined with RNA-Seq information to take a “snap shot” of the central dogma of the system. One embodiment of this aspect of the disclosure is described in detail in Example 4.

3. Cell Surface Marker Analysis

The disclosure provides methods of massively multiplexing the identification and/or quantification of cell surface markers (for example, cell surface proteins) by nucleic acid barcoding. The target molecule can be a cell surface marker associated with a barcode receiving adapter. For example, a cell surface marker can be attached to an oligonucleotide barcode receiving adapter that includes an overhang capable of accepting a portion of a barcode. The barcode can, for example, include a corresponding overhang capable of hybridizing to the overhang on the barcode receiving adapter. Thus, a plurality of cell surface markers can be tagged with barcodes. The barcode receiving adapter can also function as a further identifier. For example, a cell in a discrete volume can express cell surface markers which are then associated with barcode receiving adapters. One or more barcodes can then be attached to each barcode receiving adapter. For example, all barcode receiving adapters in a discrete volume and/or associated with cell surface markers expressed by a particular cell can receive identical barcodes. In certain embodiments, each barcode receiving adapter is distinct to the individual cell surface marker. In alternate embodiments, the cell surface markers expressed by a particular cell all receive identical barcode receiving adapters.

In one embodiment, a plurality of cells (for example, B-cells) each expressing a distinct cell surface marker (for example, distinct antibodies) is mixed with a plurality of target epitopes fused with, for example, encoded polypeptide identifier elements. The target epitopes can be allowed to bind to the cell surface markers, and then excess target epitopes removed by washing. Each cell, along with its expressed cell surface markers and any bound target epitopes, can then be encapsulated in a discrete volume (for example, an emulsion droplet), and then lysed, thus releasing the cell surface markers. The encoded polypeptide identifier elements can then be cleaved from the bound target epitopes, pooled (for example, by breaking the emulsion), and then separated from the mixture using, for example, antibodies specific to affinity tags present in the encoded polypeptide identifier elements, as described herein. The encoded polypeptide identifier elements can be further sorted by, for example, linker length and/or hydrophobicity, using HPLC, as described herein. Nucleic acid barcodes associated with the encoded polypeptide identifier elements can then be isolated and sequenced to determine the presence of particular epitopes. In certain embodiments, each discrete volume receives a origin-specific barcode, which can, for example, be ligated to the nucleic acid barcodes associated with the encoded polypeptide identifier elements, such that sequencing the resultant concatemer reveals which epitopes were present in which discrete volumes. In one embodiment, the origin-specific barcodes can also ligate to nucleic acids encoding the cell surface markers (for example, mRNAs expressed by the cell and/or cDNAs generated from such mRNAs). As such, the cell surface marker-encoding nucleic acids can be sequenced with the barcodes associated with the encoded polypeptide identifier elements, each of which is tagged with a origin-specific barcode, thus allowing combinations of particular cell surface markers binding particular epitopes to be determined by the sequencing reads.

In addition, a cell surface marker and a nucleic acid encoding the cell surface marker (for example, an mRNA) can be associated with identical barcode receiving adapters and/or barcodes. In certain embodiments, a cell that does not express a particular cell surface marker can be identified if one or more barcodes are detected in association with the cell without also detecting the mRNA corresponding to the cell surface marker.

In another example of a cell surface marker-related application, epitopes of interest (for example, epitopes from HIV, ebola virus, etc.) are labeled with nucleic acid barcodes that are specific for each epitope. Optionally, a unique molecular identifier is associated with, for example, each epitope-specific nucleic barcode. Thus, each epitope-specific nucleic acid barcode for HIV epitope #1, for example, would be associated with a different unique molecular identifier, for quantification. The labeled epitopes are mixed in bulk solution with B cells expressing antibodies on their surfaces, where they bind. Excess proteins, including unbound epitopes, are then washed from the B cells, which are encapsulated in a discrete volume, such as an emulsion droplet. Origin-specific nucleic acid barcodes are then used to label the epitope-specific nucleic barcodes, as well as any RNA of interest, for example, RNA encoding heavy and light chains of the antibody produced by the B cell. By sequencing the origin-specific nucleic acid barcodes, as well as the epitope-specific nucleic acid barcodes, antibodies that bind to each particular epitope of interest can be identified. Furthermore, sequencing RNAs associated with origin-specific nucleic acid barcodes enables the identification of antibody nucleic acid sequences that can be used to make and/or further characterize antibodies of interest.

4. Labeling Wild-Type Cells

The proteomes of wild-type cells (for example, cells not containing an expression construct or transgene useful for expressing a target protein fused to an encoded polypeptide identifier element) can be analyzed using nucleic acid identifiers that can be conjugated to polypeptides expressed by the wild-type cells. For example, nucleic acid identifiers can be attached to cysteine residues in polypeptides expressed by the wild-type cells. These nucleic acid identifiers can be, for example, barcodes designed to shift peaks of fractionated proteomes that include particular proteins as detected by, for example, mass spectrometry (MS) in predictable ways.

In more detail, one or more cells within a particular discrete volume can have its proteome labeled with origin-specific barcodes. Proteomes from different discrete volumes can then be pooled (after optional enrichment), and fractionated by, for example, HPLC. Peaks corresponding to proteins of interest, tagged with origin-specific barcodes, can be detected by, for example, MS, and isolated. The isolated fractions can be individually analyzed with respect to proteins of interest, based on sequencing of origin-specific barcodes. Alternatively, fraction-specific barcodes can be added to the origin-specific barcodes within the fraction, resulting in the formation of a barcode concatemer that provides information regarding discrete volume source and protein identity, based on fractionation. In the event that additional processing is desired (for example, exposure to different conditions and/or further fractionation methods), barcodes specific to the particular type of additional processing step can be added. After all desired processing and fractionating is complete, barcodes (or barcode concatemers) can be cleaved off of the proteins for sequencing.

In certain embodiments, a pool of samples (for example, one or more cells in a discrete volume) can include about 1,000,000 samples (for example, at least about 2, 5, 10, 50, 100, 250, 500, 1,000, 5,000, 10,000, 50,000, 100,000, 500,000, or 1,000,000 samples).

5. Affinity Analysis

The methods of the disclosure can be used to determine the binding affinity between a target molecule and another molecule (for example, a specific-binding agent). For example, an equilibrium constant (Kd) and an off-rate (k_{off}) for the binding interaction between a target molecule and another molecule (for example, a specific-binding agent) can be measured using methods known in the art. For example, if a specific-binding agent is attached to a solid support (for example, a column, chip, surface, or bead), Kd can be measured by titrating in various amounts of the target that is conjugated to a barcode. After incubating, the solid support can be washed, and the barcode can be cleaved and sequenced to determine the quantity of target that is bound to the binding moiety. This assay could be performed in reverse with the target molecule bound to the solid support and the specific-binding agent conjugated to the barcode. An alternate method involves a “sandwich” format for bulk affinity purification or analysis, in which the target molecule is complexed with a specific-binding agent (for example, a specific-binding agent labeled with a barcode, such as a origin-specific barcode) and also complexed with another specific-binding agent that is bound to a solid support. In this method, the target molecule may not be directly attached to a origin-specific barcode, but is rather labeled by binding to the specific-binding agent labeled with the origin-specific barcode.

These assays can also be performed in a competitive format by adding a molecule that will compete with the target molecule for binding to the specific-binding agent. The competitor can be added simultaneously with the target molecule, or after the addition and subsequent complexation of the target molecule to the specific-binding agent. The competitor can optionally be conjugated to a barcode. For example, the competitor can be a target molecule, for example, a target molecule labeled with a origin-specific barcode. In some instances, k_{off} can be determined by adding a non-barcoded competitor molecule capable of competing with the target molecule for binding to the specific-binding agent. The target molecules that remain bound to the support can be isolated, and barcodes associated with the target molecules can be sequenced to determine the quantity of target molecules that remained bound to the support.

An exemplary method for measuring binding affinity includes binding tagged target molecule-specific-binding agent complexes to a support (for example, via a biotin moiety attached to one component of the complex). The support can then be exposed to one or more wash conditions. The barcodes associated with the target molecules removed in each wash can be sequenced separately to determine the abundance of that unbound fraction. The barcodes can also be cleaved off the complexes still bound to the support after the washes to determine the abundance of the bound fraction. In certain embodiments, the bound fraction is permitted to remain bound to the support for about one to two days. The relative abundance of unbound fractions and the bound fraction can be compared to determine the dissociation rate. Multiple rounds of washes can be performed in this manner to generate a Kd curve. In an alternative embodiment, a specific-binding agent can be used to isolate a fraction of a plurality of target molecules (for example, by immunoprecipitation using an antibody as a specific-binding agent). This bound fraction, or a portion thereof, can be subsequently washed from the specific-binding agent. Barcodes associated with the target molecules can be isolated from the bound and unbound fractions. The relative abundance of the target molecule in the bound and unbound fractions can then be calculated to determine an affinity measurement. Multiple rounds of washes can be performed, with each subsequent wash removing an additional portion of the bound fraction, and the associated nucleic acid barcodes sequenced to generate an affinity curve. In one embodiment, the target molecules are bound to the specific-binding agent for about one to two days prior to washing.

6. Genotype-Phenotype Coupling

The disclosure further provides methods for co-detecting expressed target molecules (for example, a polypeptide, protein, protein complex, or any other gene product) and nucleic acids (for example, RNA or DNA) encoding the target molecules. In certain embodiments, a plurality of discrete volumes each contains one or more cells that are allowed to express a target polypeptide molecule. After expression and optional cell lysis, the expressed polypeptide is labeled with a origin-specific nucleic acid barcode by maintaining the discrete volume under conditions permitting the barcode labeling reaction to proceed. Nucleic acid molecules (for example, RNA or cDNA reverse transcribed therefrom) encoding the target polypeptide molecule are also labeled with a origin-specific nucleic acid barcode.

The origin-specific nucleic acid barcodes used to label a target polypeptide molecule and a corresponding nucleic acid molecule within a particular discrete volume are typically matched nucleic acid barcodes. Matched nucleic acid barcodes can be, for example, at least 80% identical (for example, 80%, 85%, 90%, 95%, 99%, or 100% identical). In certain embodiments, the matched nucleic acid barcodes are 100% identical. In other embodiments, the matched barcodes may have different sequences but can be identified as members of a matched pair based on their sequences (for example, by specifying in advance that two particular barcodes are introduced into the same container, such that molecules tagged with the two particular barcodes must have originated from the same container). Origin-specific nucleic acid barcodes can optionally be introduced into a discrete volume bound to a common support, such as a bead as described elsewhere herein. In such instances, origin-specific nucleic acid barcodes intended for binding to the target polypeptide molecule can be comprised within a tagging element that includes an specific binding agent (for example, an antibody) specific for the target polypeptide molecule, while origin-specific nucleic acid barcodes intended for binding to the corresponding nucleic acid molecule can be comprised within a tagging element that includes an specific binding agent (for example, a nucleic acid molecule) specific for the corresponding nucleic acid molecule.

After labeling with origin-specific nucleic acid barcodes, target polypeptide molecules and/or corresponding nucleic acids can be combined to form a pool. The discrete volume of origin for a given target polypeptide molecule or nucleic acid in the pool can be determined by sequencing its associated origin-specific nucleic acid barcode. Particular portions of the pool can optionally be isolated. For example, in the case of the target polypeptide molecule being an antibody or a fragment thereof, chromatography on a column including immobilized antigen to which the antibody binds can be carried out, optionally under particular or varying conditions of stringency to permit the isolation of, for example, antibodies having particularly high binding affinity. In another example, in the case of a target polypeptide molecule being an antigen comprising an epitope, the chromatography can be carried out using a column including immobilized antibody (or an antigen-binding fragment thereof). In other examples, an activity or property other than binding affinity can be assessed. In any case, particular target polypeptide molecules with desirable features can be isolated and the identities of the target polypeptide molecules can be determined by sequencing of the origin-specific nucleic acid barcodes. The genotype corresponding to the phenotype of selected target polypeptide molecules can be determined by use of sequencing to identify matched origin-specific nucleic acid barcodes in pooled nucleic acid samples, and then optionally sequencing the nucleic acids attached to these barcodes.

7. Reporter Cells

The methods of the disclosure can be used to analyze properties of target molecules, for example, target polypeptide molecules. For example, target molecules may induce responses in cells. In some instances, a target molecule can be a soluble signal (for example, a secreted protein, peptide, small molecule, or other specific-binding agent) capable of interacting with a cell (for example, by binding to a cell surface receptor), thereby inducing a downstream effect in the cell. Exemplary downstream effects include, without limitation, changes (for example, increases or decreases) in gene expression (for example, changes in mRNA expression level), changes in intracellular signaling pathways, and/or activation or inhibition of cellular activities (for example, cell proliferation, cell growth, cell death, change in cell morphology, and/or change in cell motility).

Thus, a discrete volume of the methods of the disclosure can, in some embodiments, include one or more reporter cells in which such downstream effects can be induced by a target molecule. For example, expression of one or more mRNAs by a reporter cell can be altered (for example, increased or decreased) by direct or indirect interaction with the target molecule. Such an mRNA may, for example, encode a target molecule, or alternatively may not encode a target molecule. An mRNA can, in some instances, encode an encoded polypeptide identifier element. In certain embodiments, the mRNAs are labeled with origin-specific barcodes (for example, the same origin-specific barcode used to label the target molecule). For example, the mRNAs or corresponding cDNAs can be ligated to origin-specific barcodes. The labeled mRNAs or cDNAs can subsequently be obtained (for example, by lysing the reporter cell), and sequenced to determine how the expression of the mRNA in the reporter cell was altered by the target molecule (for example, by determining the quantity of each distinct mRNA in a particular discrete volume). In certain embodiments, the reporter cell is lysed, and the mRNAs labeled with origin-specific barcodes from multiple discrete volumes are pooled (for example, with the labeled target molecules or labeled tags), amplified, and sequenced. Labeled mRNAs may be, for example, converted to labeled cDNAs prior to pooling, amplifying, and/or sequencing. In particular embodiments, the entire transcriptome of the reporter cell is labeled with origin-specific barcodes and sequenced according to RNA-seq methods known in the art.

8. Combinatorial Chemistry

The disclosure provides methods for coupling a target molecule to a plurality of nucleic acid barcodes, for example, in the form of a concatemer of barcodes. Each barcode can be, for example, associated with a particular condition, such that the set of conditions to which the target molecule is exposed can be determined by sequencing the barcodes. In some embodiments, each barcode is added to a growing barcode concatemer as the target molecule is exposed to the condition with which the barcode is associated, such that sequencing the barcode concatemer reveals the order in which the target molecule was exposed to each condition. The barcodes can be associated with an specific binding agent, which can recognize the target molecule and thus facilitate coupling of the barcode and target molecule. In certain embodiments, the target molecule is exposed to a plurality of compounds (for example, small molecules, nucleic acids, peptides, polysaccharides, or combinations thereof), and each of the compounds is associated with a distinct nucleic acid barcode, which can be coupled to the target molecule in turn (for example, by adding each barcode to a growing barcode concatemer). For example, a target molecule can be exposed to a set of reaction conditions, each featuring a particular compound, in which each exposure results in addition of a distinct barcode to the target molecule. Thus, the compounds to which the target molecule was exposed, and the order of their exposure, can be determined afterward by sequencing the barcodes associated with the target molecule.

### D. Compositions and Kits

The present disclosure also concerns compositions and kits that can be used in carrying out the methods of the disclosure. In one example, a disclosed composition includes a barcode labeling complex. The barcode labeling complex includes a solid or semi-solid substrate (such as a bead, for example a hydrogel bead), and a plurality of barcoding elements reversibly coupled thereto, wherein each of the barcoding elements comprises an indexing nucleic acid identification sequence (such as RNA, DNA or a combination thereof) and one or more of a nucleic acid capture sequence that specifically binds to target nucleic acids and a specific binding agent that specifically binds to the target molecules. In some examples, the origin-specific barcode further includes one or more cleavage sites. In some examples, at least one cleavage site is oriented such that cleavage at that site releases the origin-specific barcode from a substrate to which it is coupled. In some examples, at least one cleavage site is oriented such that the cleavage at the site releases the origin-specific barcode from the target molecule specific binding agent. In some examples, the origin-specific barcode further comprises one or more capture moieties, covalently or non-covalently linked. In certain example, the one or more capture moieties comprises biotin, such as biotin-16-UTP. In some examples, the origin-specific barcode further comprises one or more of a sequencing adaptor or a universal priming sites. In some examples, the target molecule specific binding agent comprises an antibody or a fragment thereof, a polypeptide or peptide comprising an epitope recognized by the target molecule, or a nucleic acid. In some examples, each of the origin-specific barcodes comprises one or more indexes, one or more sequences that enable gene specific capture and/or amplification, and/or one or more sequences that enable sequencing library construction. In some examples each of the barcodes comprises four indexes, which can be combinatorially assembled. In some examples, the sequences that enable sequencing library construction comprises an Illumina P7 sequence and/or an Illumina sequencing primer. In some examples, the barcodes comprises a primer for DNA synthesis, such as a primer suitable for DNA synthesis on a DNA template or an RNA template. Also disclosed are kits, that can include any and all of the compositions disclosed herein.

The invention is further defined with reference to the following numbered clauses:

1. A method multiplex analysis of polypeptides in a sample, comprising

providing a sample comprising cells, or an acellular system, comprising one or more target polypeptides of interest and/or nucleic acids encoding target polypeptides of interest, optionally linking a peptide identifier element to the target polypeptides to allow multiplex characterization of the presence and/or amount in the individual polypeptides of interest;

segregating cells, single cells or a portion of the acellular system from the sample into individual discrete volumes; and

labeling the target polypeptides in the discrete volumes with origin-specific barcodes present in the discrete volumes to create origin-labeled target polypeptides, wherein the origin-labeled target polypeptides from each individual discrete volume comprise the same unique indexing nucleic acid identification or matched sequence; and

detecting the nucleotide sequence of the origin-specific barcodes, thereby assigning the set of target polypeptides to a specific discrete volume, while maintaining information about sample origin of the target polypeptides; and/or

labeling the target polypeptides with distinguishable mass tags to create mass tag labeled target polypeptides, wherein the mass tag labeled target polypeptides comprises mass tag matched to the target polypeptide and/or mass tags matched to the peptide identifier element; and

detecting the mass tags, thereby detecting the target polypeptides.

2. The method of clause 1, further comprising;

assigning the set of target polypeptides to target nucleic acids in the sample or set of samples while maintaining information about sample origin of the target polypeptides and target nucleic acids;

labeling the target nucleic acids in the discrete volumes with the origin-specific barcodes present in the discrete volumes to create origin-labeled target nucleic acids, wherein the origin-labeled target nucleic acids from each discrete volume comprise the same, or matched, unique indexing nucleic acid identification sequence as the origin-labeled target polypeptides; and

detecting the nucleotide sequence of the origin-specific barcodes, thereby assigning the set of target polypeptides to target nucleic acids in the sample or set of samples while maintaining information about sample origin of the target polypeptides and the target nucleic acids.

3. The method of clause 2, wherein detecting the nucleotide sequence of the origin-specific barcodes comprises nucleic acid sequencing, amplification, hybridization, or any combination thereof.
4. The method of any one of clauses 1-3, wherein detecting the mass tags comprises mass spectral analysis, chromatography or a combination thereof.
5. The method of any one of clauses 1-4, wherein the nucleic acids encoding the target polypeptides are operatively connected to a nucleic acid sequence encoding the peptide identifier element.
6. The method of any one of clauses 1-5, wherein the target polypeptides are covalently linked to the polypeptide identifier element, optionally by a peptide linker.
7. The method of any one of clauses 1-6, wherein the target polypeptides and the polypeptide identifier element, and optionally the peptide linker, comprise a single polypeptide.
8. The method of any one of clauses 1-7, wherein individually the target polypeptides, optionally the peptide linker, and the polypeptide identifier element are encoded by a single target nucleic acid sequence operatively connected to a promoter.
9. The method of any one of clause 9, wherein the single target nucleic acid sequence encodes an amber codon positioned between the target polypeptide coding sequence and the polypeptide identifier element.
10. The method of any one of clauses 1-4, wherein the target polypeptides are non-covalently linked to the polypeptide identifier element,
11. The method of any one of clauses 1-10, wherein the cells of the sample comprise an amber suppressor tRNA expression system, such that the target polypeptide does not express the polypeptide identifier element in the absence of expression of the amber suppressor tRNA and wherein expression of the tRNA causes the target peptide to be expressed with the molecular identifier element.
12. The method of any one of clauses 1-11, wherein the polypeptide identifier element is located C-terminal to the target polypeptide, N-terminal to the target peptide or internal to the target peptide.
13. The method of any one of clauses 1-12, wherein the polypeptide identifier element comprises one ore more affinity tags.
14. The method of any one of clause 13, wherein the affinity tag comprises a FLAG tag, HA tag, His tag, Myc tag, AU1 tag, T7 tag, OLLAS tag, Glu-Glu tag, V5 tag, VSV tag, a fluorescent protein, or a combination thereof.
15. The method of any one of clauses 13-14, wherein the different target polypeptides are linked to identical affinity tags.
16. The method of any one of clauses 13-14, wherein the different target polypeptides are linked to different affinity tags.
17. The methods of any one of clause 13-16, wherein the polypeptide identifier elements comprise between about 2 and about 200 different affinity tags.
18. The method of any one of clauses 1-17, wherein the polypeptide identifier element further comprises one or more peptide barcodes.
19. The method of clause 18, wherein the polypeptide identifier element comprises between about 2 and about 200 different peptide barcodes.
20. The method of any one of clauses 1-19, wherein the polypeptide identifier element comprises one or more affinity tags and one or more peptide barcodes.
21. The method of any one of clauses 18-20, wherein the different peptide barcodes have different physical characteristic, allowing separation of the different peptide barcodes optionally in conjunction with the affinity tags.
22. The method of clause 21, wherein the different physical characteristic comprises one or more of amino acid sequence, sequence length, charge, size, molecular weight, hydrophobicity, or other separable property.
23. The method of any one of clauses 1-22, wherein one or more of the polypeptide identifier element, the affinity tag, or the peptide barcode comprises an attachment site for the origin specific bar code.
24. The method of clause 23, where the origin specific barcode is covalently attached to the attachment site.
25. The method of any one of clauses 23-24, wherein the attachment site is at the C-terminus of the polypeptide identifier element, the affinity tag, or the peptide barcode.
26. The method of any one of clauses 23-25, wherein the attachment site comprise an amino acid side chain.
27. The method of clause 26, wherein the amino acid side chain comprises a cysteine sidechain.
28. The method of any one of clauses 1-25, wherein the polypeptide identifier comprises a peptide cleavage site positioned between the target polypeptide and the polypeptide identifier element.
29. The method of clause 26, further comprising cleaving the polypeptide identifier element from the target polypeptide.
30. The method of clause 10, wherein non-covalent linkage comprises a specific binding agent.
31. The method of any one of clauses 1-30, wherein labeling the target polypeptides in the discrete volumes with origin-specific barcodes, comprises contacting the target polypeptides present in the discrete volumes with a specific biding agent that specifically binds to the peptide identifier element, wherein the specific biding agent that specifically binds to the peptide identifier element is linked to the origin-specific barcode.
32. The method of any one of clauses 1-31, wherein labeling the target polypeptides in the discrete volumes with distinguishable mass tags present in the discrete volumes to create mass tag labeled target polypeptides, comprises contacting the target polypeptides present in the discrete volumes with a specific biding agent that specifically binds to the peptide identifier element, wherein the specific biding agent that specifically binds to the peptide identifier element is linked to distinguishable mass tags.
33. The method of any one of clauses 13 to 32, further comprising, labeling the affinity tag with an affinity tag-specific nucleic acid barcode.
34. The method of clause 33, wherein labeling the affinity tag with the affinity tag-specific nucleic acid barcode comprises, contacting the affinity tag with an affinity tag specific binding agent, that specifically binds the affinity tag, wherein the affinity tag specific binding agent comprises the affinity tag-specific nucleic acid barcode.
35. The method of clause 34, wherein the affinity tag specific binding agent comprises an antibody.
36. The method of clause 35, further comprising attaching an origin-specific barcode to the affinity tag-specific nucleic acid barcode.
37. The method of any one of clauses 1-36, further comprising labeling the target polypeptides with a target polypeptide specific barcode.
38. The method of clause 37, wherein labeling the target polypeptide with the target polypeptide specific barcode comprises, contacting the target polypeptide with a target polypeptide specific binding agent, that specifically binds the target polypeptide, wherein the target polypeptide specific binding agent comprises the target polypeptide specific nucleic acid barcode.
39. The method of clause 38, wherein the target polypeptide specific binding agent comprises an antibody.
40. The method of clause 39, further comprising attaching an origin-specific barcode to the target polypeptide specific nucleic acid barcode.
41. The method of any one of of 1-40, further comprising pooling the sample.
42. The method of any one of clauses 1-41, further comprising enriching for the polypeptide identifier elements.
43. The method of clause 42, wherein the enriching comprises contacting the polypeptide identifier element with immobilized protein G.
44. The method of clause 43, wherein the enriching comprises contacting the polypeptide with a specific binding agent that specifically binds the polypeptide identifier element and enriching using the specific binding agent that specifically binds the polypeptide identifier element.
45. The method of any one of clauses 1-44, further comprising enriching for one or more target polypeptides of interest.
46. The method of clause 45, wherein the enriching comprises contacting the polypeptides of interest with a specific binding agent that specifically binds the polypeptides of interest and enriching using the specific binding agent that specifically binds the polypeptide identifier element.
47. The method of any one of clauses 41-46, further comprising fractionating the pooled sample, or an enriched fraction thereof, based on one or more properties of the peptide barcodes and isolating the fractions of interest.
48. The method of clause 47, wherein the properties of the peptide barcodes comprise the length and/or hydrophobicity.
49. The method of any one of clauses 47-48, wherein the fractionation comprises chromatography.
50. The method of clause 49, wherein the chromatography comprises HPLC separation.
51. The method of any one of clauses 47-50, wherein the fractionation comprises performing liquid chromatography-mass spectrometry (LC-MS) on the pool, or a partially purified fraction thereof, and isolating one or more peaks corresponding to one or more labeled target polypeptides to be isolated.
52. The method of clause 51, wherein each of the sample-specific barcodes is designed to shift the peak associated with a target polypeptide in a mass spectrometry profile by a predictable mass different corresponding to different isotopes of a particular atom or atoms, or a different peptide tag.
53. The method of clauses any one of 18 to 52, further comprising labeling the peptide barcode with a peptide barcode-specific nucleic acid barcode.
54. The method of clause 53, wherein labeling the peptide barcode comprises contacting the sample with a peptide barcode specific binding agent that specifically binds the peptide barcode, wherein the peptide barcode specific binding agent comprises a peptide barcode-specific nucleic acid barcode.
55. The method of any one of clauses 53-54, further comprising attaching the origin-specific barcode to the peptide barcode-specific nucleic acid barcode.
56. The method of any one of clauses 53-55, wherein the sequence of the peptide barcode-specific nucleic acid barcode is correlated to the length of the peptide barcode
57. The method of any one of clause 1-56, further comprising isolating labeled target polypeptides.
58. The method of clause 57, wherein isolating a population of target polypeptides, is based on one or more properties of one or more of the target polypeptides.
59. The method of any one of clauses 1-58, wherein the barcodes comprise RNA, DNA, or a combination thereof.
60. The method of any one of clauses 1-59, wherein the origin-specific barcodes and/or distinguishable mass tags are reversibly coupled to a solid or semisolid substrate.
61. The method of any one of clauses 1-60, further comprising encapsulating the single cells or a portion of the acellular system from the sample with a bead comprising the origin-specific barcodes reversibly coupled thereto.
62. The method of any one of clauses 1-61, wherein the origin-specific barcodes further comprise a nucleic acid capture sequence that specifically binds to the target nucleic acids and/or a specific binding agent that specifically binds to the target polypeptides.
63. The method of clause 62, wherein the origin-specific barcodes comprise two or more populations of origin-specific barcodes, wherein a first population comprises the nucleic acid capture sequence and a second population comprises the specific binding agent that specifically binds to the target polypeptides.
64. The method of any one of clauses 1-63, wherein the origin-specific barcode further comprises a capture moiety, covalently or non-covalently linked.
65. The method of any one of clauses 1-64, wherein the origin-specific barcode further comprise a sequencing adaptor.
66. The method of any one of clauses 1-65, wherein the origin-specific barcode further comprises universal priming sites.
67. The method of any one of clauses 1-66 further comprising, selectively isolating the origin-labeled polypeptides and origin-labeled nucleic acids.
68. The method of clause 67, wherein isolating the origin-labeled polypeptides and origin-labeled nucleic acids comprises capturing the origin-specific barcode via the capture moiety.
69. The method of clause 68, wherein the one or more capture moieties is captured with a capture moiety specific binding agent that specifically binds to the one or more capture moieties.
70. The method of clause 69, wherein the one or more capture moieties is captured on a solid support.
71. The method of any one of clauses 67-68, wherein the capture moiety specific binding agent is attached to the solid support.
72. The method of any one of clauses 64-71, wherein the one or more capture moieties comprises biotin.
73. The method of any one of clauses 69-72, wherein the capture moiety specific binding agent comprises streptavidin.
74. The method of any one of clauses 1-73, wherein the origin-specific barcode comprises biotin-16-UTP.
75. The method of any one of clauses 1-74, wherein the origin-specific barcode further comprises a primer-specific region.
76. The method of any one of clauses 1-75, wherein each of the origin-specific barcodes further comprises a unique molecular identifier.
77. The method of any one of clauses 1-76, wherein each of the origin-specific barcodes comprises one or more indexes, one or more sequences that enable gene specific capture and/or amplification, and/or one or more sequences that enable sequencing library construction.
78. The method of any one of clauses 1-77, wherein the discrete volume comprises an aqueous droplet in an emulsion.
79. The method of clause 78, wherein the emulsion comprises one or more surfactants, thereby stabilizing the emulsion.
80. The method of clause 79, wherein the one or more surfactants comprises one or more fluorinated surfactants.
81. The method of any one of clauses 79-80, wherein the emulsion comprises a continuous phase and the continuous phase of the emulsion comprises a fluorinated oil.
82. The method of any one of clauses 78-81, further comprising, breaking the emulsion, thereby pooling the contents of the discrete volumes.
83. The method of any one of clauses 1-82, wherein the origin-specific barcode further comprises one or more cleavage sites.
84. The method of clause 83, wherein at least one cleavage site is oriented such that cleavage at that site releases the origin-specific barcode from a substrate to which it is coupled.
85. The method of clause 84, wherein the substrate comprises the bead.
86. The method of any one of clauses 83-85, wherein at least one cleavage site is oriented such that the cleavage at the site releases the origin-specific barcode from the target polypeptide specific binding agent.
87. The method of any one of clauses 1-86, further comprising lysing the cells.
88. The method of any one of clauses 1-87, wherein the target polypeptides, optionally in association and the target nucleic acids are produced by the individual cells in the discrete volumes.
89. The method of any one of clauses 1-88, wherein the cell is a wild-type cell.
90. The method of any one of clauses 1-89, wherein each of the polypeptides comprises a cysteine residue.
91. The method of clause 90, further comprising attaching of the origin-specific nucleic acid barcodes to the cysteine residues.
92. The method of any one of clauses 1-91, further comprises cleaving the origin-specific barcode from the labeled target polypeptides.
93. The method of any one of clauses 1-92, further comprising, exposing isolated labeled target polypeptides to a reaction condition and attaching a condition-specific barcode to each of the sample-specific barcodes labeling the isolated labeled target polypeptides, thereby forming a conjugate barcode.
94. The method of clause 93, further comprising attaching further condition-specific barcodes to the origin-specific barcodes, wherein each distinct condition-specific barcode is associated with a distinct condition, such as exposure to a particular temperature, pH, small molecule, nucleic acid, peptide, carbohydrate, lipid, solute concentration, solvent, or filter.
95. The method of any one of clauses 1 to 94, wherein the target polypeptides represent a library of randomly mutated polypeptides or non-randomly mutated polypeptides.
96. The method of any one of clauses 1-95, wherein the sample comprises one or more cells.
97. The method of any one of clauses 1-95, wherein samples comprises the acellular system of target polypeptides and target nucleic acids.
98. The method of clause 97, acellular system of target polypeptides and target nucleic acids comprises a cell-free extract or a cell-free transcription and/or cell-free translation mixture.
99. The method of any one of clauses 1 to 98, wherein the sample comprises one or more synthetic genetic constructs comprising one or more polypeptide coding sequence operably connected to a promoter.
100. The method of clause 99, wherein the one or more synthetic genetic constructs comprises a set of synthetic genetic constructs, which optionally are comprised of combinatorially generated parts.
101. The method of any one of clauses 97-100, wherein the one or more synthetic genetic constructs comprises, the peptide coding sequences for a biosynthetic pathway.
102. The method of any one of clauses 97-101 wherein the one or more synthetic genetic constructs comprises, the peptide coding sequences for a gene cluster.
103. The method of any one of clauses 91-102, wherein the cells are transformed or transfected with one or more synthetic genetic constructs.
104. The method of any one of clauses 1-103, further comprising synthesizing cDNAs from the target nucleic acids, wherein the cDNA comprises the nucleic acid sequence of the target nucleic acid, or a fragment thereof and the sequence of the origin-specific barcode.
105. The method of clause 104, wherein the origin-specific barcodes are primers for the cDNA synthesis.
106. The method of any one of clauses 1-105, wherein the target nucleic acid comprises mRNA or cDNA.
107. The method of any one of clauses 1-106, further comprising determining the sequence of the target nucleic acids or a portion thereof.
108. The method of any one of clauses 1-107, wherein the target nucleic acid, or complement thereof, encodes a target polypeptide.
109. The method of any one of clauses 1-108, wherein linking the polypeptides of interest to the peptide identifier elements, comprises inserting the nucleic acid sequence encoding peptide identifier elements into the genome of a cell, wherein the nucleic acid sequence encoding the polypeptides of interest to the nucleic acid sequence encoding peptide identifier elements are operatively linked.
110. The method of clause 109, wherein inserting the nucleic acid sequence encoding peptide identifier elements comprises using a genome editing system
111. The method of clause 110, wherein the genome editing system comprises a CRISPR system.
112. The method of any one of clauses 1 to 111, wherein one or more of the discrete volumes further comprises:

a reporter cell expressing one or more mRNAs; where the quantity of one or more of the mRNAs expressed by the reporter cell within a discrete volume varies in response to the presence, amount, or activity of a target polypeptide in the discrete volume and wherein the mRNA or corresponding cDNA in a discrete volume is labeled with the same origin-specific nucleic acid barcode as the target polypeptide in the discrete volume.

The following examples are intended to illustrate, but not limit, the invention.

### EXAMPLES

### Example 1—Proteomics Method Utilizing Encoded Polypeptide Identifier Elements Having Distinct Affinity Tags

In one example of the proteomics methods of the disclosure, which is illustrated in FIG. 4, each member of a set of target proteins (P1-10) that is expressed within a discrete volume (for example, a well or a droplet) is fused to a protease-cleavable polypeptide identifier element including a distinct affinity tag (AU1, T7, etc.). After protein expression and cleavage of polypeptide identifier elements, antibodies specific for each of the distinct affinity tags, and which include affinity molecule-specific nucleic acid barcodes, are added. Origin-specific nucleic acid barcodes are then added by PCR or ligation to the affinity tag-specific nucleic acid barcodes. The resulting nucleic acid barcode concatemers each include information about the affinity tag (and, by extension, the identity of the target protein) and the discrete volume of origin. After the polypeptide identifier elements are labeled in this manner, the contents of multiple discrete volumes can be pooled and purified (for example, by binding to protein G), and then nucleic acid barcode concatemers from the pool can be sequenced to determine the quantity of each target protein from each discrete volume.

This method can be varied in a number of different ways by the incorporation of different steps and features as described herein. For example, the method can include the co-labeling of RNA or cDNA corresponding to target proteins with origin-specific nucleic acid barcodes to permit DGE analysis. In addition, C-terminal cysteines can be used for the linking of origin-specific nucleic acid barcodes to polypeptide identifier elements, or protein G binding sites can be encoded within polypeptide identifier elements to facilitate enrichment/purification after nucleic acid barcode addition, as explained above. Also as explained above, the nucleic acid barcodes can include unique molecule identifiers (UMI's), which can be used to normalize samples for variable amplification efficiency (see above).

### Example 2—Proteomics Method Utilizing Encoded Polypeptide Identifier Elements Including an Affinity Tag and Variable Length Peptide Barcodes

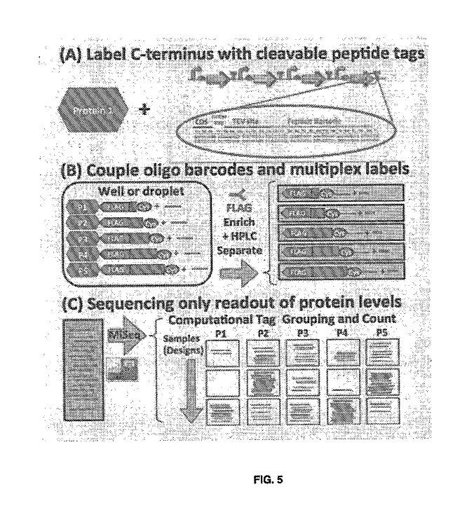

In another example of a proteomics method of the disclosure, which is illustrated in FIG. 5, each member of a set of target proteins (P1-P5) that is expressed within a discrete volume (for example, a well or a droplet) is fused to a protease-cleavable polypeptide identifier element including an affinity tag (for example, a FLAG tag) and a distinct peptide barcode. As shown in FIG. 5, the distinct peptide barcodes of different peptides tags within a discrete volume can vary by length. In addition, each polypeptide identifier element can include a C-terminal cysteine, to which an origin-specific nucleic acid barcode can be attached. After protein expression, origin-specific nucleic acid barcodes are added. Pooled polypeptide identifier elements are enriched using, for example, affinity tag-specific antibodies (for example, anti-FLAG antibodies). Cleavage of the polypeptide identifier elements can occur before or after affinity tag-based enrichment. Distinct polypeptide identifier elements can then be separated from one another based on peptide barcode length using, for example, high-performance liquid chromatography (HPLC). Each HPLC fraction can receive a distinct fraction-specific nucleic acid barcode, which can be ligated to the origin-specific nucleic acid barcodes. The resultant concatemers can then be pooled and sequenced, thus providing information as to which target proteins, at what levels, were expressed in each discrete volume.

This method can also be varied in a number of different ways by incorporation of different steps and features as described herein. For example, the method can include the co-labeling of cDNA corresponding to target proteins with origin-specific nucleic acid barcodes to permit DGE analysis. In addition, rather than C-terminal cysteines, affinity tag-specific antibodies can be used for the linking of origin-specific barcodes to polypeptide identifier elements (as explained in Example 1) and/or protein G binding sites can be encoded within polypeptide identifier elements to facilitate enrichment/purification after nucleic acid barcode addition, as explained above. Moreover, the placement of an affinity tag and peptide barcode in the polypeptide identifier element (whether on the N- or C-terminal end) can vary. Also, as explained above, the nucleic acid barcodes can include unique molecule identifiers (UMI's), which can be used to normalize samples for variable amplification efficiency (see above).

### Example 3—Proteomics Method Utilizing Encoded Polypeptide Identifier Elements Including Multiple Affinity Tags and Variable Length Peptide Barcodes

In a further example of the proteomics methods of the disclosure, which is illustrated in FIG. 6, the encoded polypeptide identifier elements include both distinct affinity tags and variable length peptide barcodes, thus allowing each encoded polypeptide identifier element to be distinguished by two different means. Here, each member of a set of target proteins (P1-P10) that is expressed within a discrete volume (for example, a well or a droplet) is fused to a protease-cleavable polypeptide identifier element having a distinct combination of affinity tag and variable length peptide barcode, such that no two distinct target proteins within a discrete volume receive polypeptide identifier elements that have the same peptide barcode length and the same affinity tag. As such, the target proteins can be distinguished by their encoded polypeptide identifier elements.

After protein expression, origin-specific nucleic acid barcodes are added, which can bind to terminal cysteine residues (for example, C-terminal cysteine residues) on the polypeptide identifier elements. Optionally, after polypeptide identifier element cleavage, origin-specific nucleic acid barcode-labeled polypeptide identifier elements are pooled and then are separately enriched for using affinity tag-specific antibodies (for example, anti-FLAG and anti-His antibodies, as illustrated). Enriched samples can be labeled with affinity tag-specific nucleic acid barcodes. After cleavage of the encoded polypeptide identifier elements from the target proteins, antibodies can bind to the appropriate affinity tags. The origin-specific nucleic acid barcodes can also be attached to C-terminal cysteine residues at this time. In addition, affinity tag-specific nucleic acid barcodes can be dissociated from their respective antibodies and attached to the origin-specific nucleic acid barcodes. For example, the affinity tag-specific nucleic acid barcodes can be attached to the origin-specific nucleic acid barcodes prior to or after attachment of the origin-specific nucleic acid barcodes to the C-terminal cysteine residues. Alternatively, the affinity tag-specific nucleic acid barcodes can be attached to the C-terminal cysteine residues.

The antibody/encoded polypeptide identifier element/origin-specific nucleic acid barcode/affinity tag-specific nucleic acid barcode complexes can then be subdivided by the length and/or hydrophobicity of the encoded polypeptide identifier element, for example, by HPLC separation. The origin-specific nucleic acid barcode-affinity tag-specific nucleic acid barcode concatemers in each HPLC fraction can then be cleaved, and the nucleic acid concatemers further barcoded with nucleic acid barcodes indicating the HPLC fraction of origin. These fraction-specific barcodes can be attached, for example, to the origin-specific nucleic acid barcode or the affinity tag-specific nucleic acid barcode. Thus, the resultant triple barcodes can be pooled, amplified, and sequenced, with each sequence indicating discrete volume of origin and the exact identity of the original protein of interest based on the affinity tag-specific nucleic acid barcode and HPLC fraction-specific nucleic acid barcode.

In alternate embodiments, the encoded polypeptide identifier elements can be subdivided by length and/or hydrophobicity (for example, by HPLC separation, as described above) prior to binding of antibodies to the affinity tags. For example, encoded polypeptide identifier elements can be cleaved from the proteins of interest and then fractionated by HPLC according to length and/or hydrophobicity. Each fraction can then receive a distinct fraction-specific nucleic acid barcode. Fraction-specific nucleic acid barcodes can be, for example, attached to terminal cysteine residues on encoded polypeptide identifier elements. Alternatively, the fraction-specific nucleic acid barcodes can be attached to origin-specific nucleic acid barcodes, for example, origin-specific nucleic acid barcodes attached to terminal cysteine residues of the encoded polypeptide identifier elements. Affinity tags associated with affinity tag-specific nucleic acid barcodes can then be used to further subdivide the encoded polypeptide identifier elements, as described above. Such affinity tags can be added to each fraction (for example, prior to or after addition of the fraction-specific barcodes). Alternatively, the fractions can be pooled after receiving fraction-specific nucleic acid barcodes, and then the affinity tags can be added to the pool. The affinity tag-specific nucleic acid barcodes can be attached to the fraction-specific nucleic acid barcodes (for example, by direct attachment, or indirectly via a origin-specific barcode), thereby forming triple labels each including a fraction-specific nucleic acid barcode, a origin-specific nucleic acid barcode, and an affinity tag-specific nucleic acid barcode. The triple labels can be pooled, amplified, and sequenced, with each sequence indicating discrete volume of origin and the exact identity of the original target protein based on the affinity tag-specific nucleic acid barcode and fraction-specific nucleic acid barcode.

### Example 4—Rapid Prototyping of Genetic Systems in Emulsion Droplets Including Cell Free Mixes

The methods of the disclosure can be used in rapid prototyping of synthetic genetic systems in the context of, for example, cell free extracts of high-value organisms. This can involve, for example, determining a set of expression levels (for example, expression levels for a set of genes of interest) and a set of part functions before down-selecting to a smaller set to be tested in vivo. In this way, a single “snapshot” of a genetic system can be used for preliminary debugging. Prototyping can be performed using a cell-free protein synthesis (CFPS) system. An exemplary CFPS system can include cell extracts associated with specific organisms rather than, for example, a defined TX-TL mix. Prototyping systems of the disclosure can be used, for example, to characterize about, for example, 100,000 constructs on the scale of, for example, minutes, as opposed to the scale of, for example, two to three constructs per year in a native host. Exemplary organisms and organelles for use as a source for CFPS include, for example, E. coli, Streptomyces, Saccharopolyspora spinosa, corn, chloroplasts, Myxococcus xanthus, lactic acid bacteria, and coryneform bacteria.

An exemplary prototyping method can involve the following procedures. Cells can be grown in, for example, liquid culture, harvested, and lysed. Cellular debris and genomic DNA can be removed, for example, by centrifugation, and a final dialysis can be performed with a suitable storage buffer. Design of experiments (DOE) can be applied to optimize the physicochemical environment and extract preparation. The CFPS can be used in micro-well plates to analyze, for example, >384 constructs at a time. Alternatively, the CFPS can be included in emulsion droplets containing, for example, individual constructs, as shown in FIG. 7. Here, plasmids containing genes prototypes (for example, prototypes assembled from one or more parts, such as promoters, coding sequences, and terminators) are encapsulated in an emulsion droplet with a bead including a plurality of attached barcode indices (for example, origin-specific barcodes) and a CFPS mix, such that each droplet receives approximately one barcoded bead and no more than one prototyping plasmid. The prototypes are transcribed into mRNA, which is optionally reverse transcribed into cDNA. The mRNA or cDNA can then be tagged with the barcodes in a origin-specific manner (for example, a distinct barcode for each emulsion droplet). A plasmid may contain more than one prototype gene, such that multiple distinct mRNAs/cDNAs are generated in a droplet from a single plasmid. In this scenario, each of the multiple distinct mRNAs/cDNAs can be labeled with the same barcode, thereby indicating the droplet from which they originated (for example, origin-specific barcoding). In some instances, the mRNA is translated into polypeptides, which can also be tagged with the origin-specific barcodes, thereby origin-specifically barcoding the polypeptides and the mRNAs/cDNAs encoding the polypeptides with the same barcodes. Barcoded mRNAs/cDNAs and/or polypeptides can be pooled, for example, by breaking the emulsion, and then sequenced to determine, for example, origin-specific transcript levels and quantification of parts (for example, promoters, coding sequences, and terminators).

In some embodiments, emulsification occurs immediately following an assembly reaction. Individual constructs can be linear or circularized. Emulsions can include, for example, aqueous droplets in fluorinated oil, for example, stabilized by a surfactant, such as a PEG-PEO fluorosurfactant. The microfluidic system used to make the droplets can include a microfluidic droplet generator that can encapsulate one synthetic DNA construct with CFPS mixes and one barcoded bead, incubate, and sort the droplets. An optimal droplet size for genetic part screening can be, for example, ˜35 μm, a droplet size that can be stable with fluorosurfactant for weeks at room temperature. Droplets may each have a volume of about 21 pL. The maximum sorting rate may be, for example, up to 1 kHz, which, for a loading efficiency of 0.1, can yield a net screening throughput of approximately 105 genetic constructs per hour. The droplets can then be recovered, chemically ruptured, and their contents used for analysis via sequencing. Sequencing can be used to, for example, determine transcript-related values, such as digital gene expression (DGE), promoter/terminator strengths, and unwanted transcription or non-functional parts. Protein levels can be measured, for example, before, concurrently, or after sequencing.

### Example 5—Optimization of Protein Barcodes

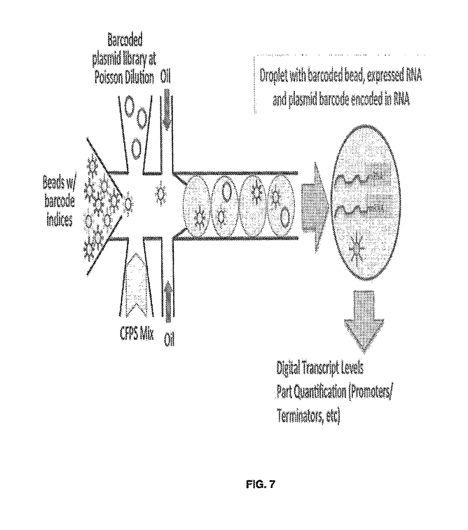

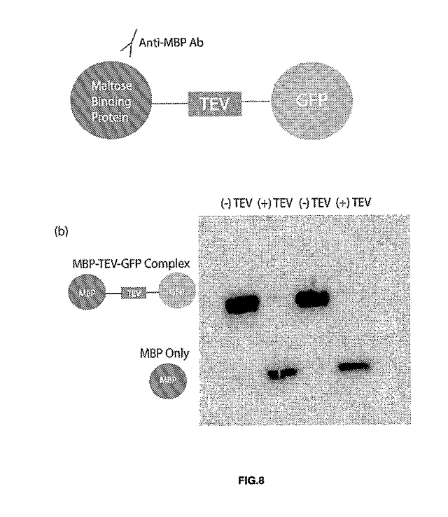

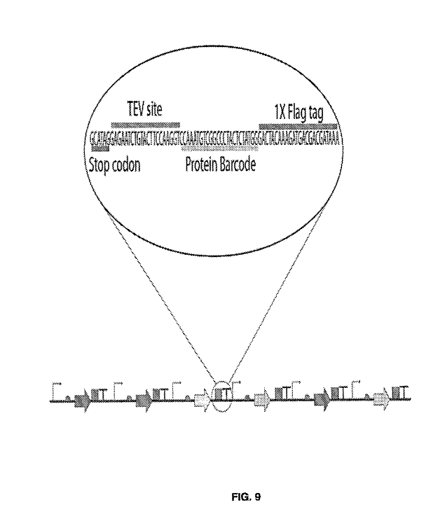

TEV digestion protocols were optimized using a Maltose Binding Protein (MBP)-GFP fusion construct containing the TEV digestion sequence between the two proteins (FIG. 8). In certain example implementations of the disclosed methods, filtration-based size selection approach is replaced with epitope-based (FLAG tag) enrichment. An example of the FLAG tag construct is shown in FIG. 9. As shown in FIG. 9 this example of a protein barcode has 23 amino acids, including a TEV digestion site (7 amino acids), a unique barcode sequence (8 amino acids), and 1X FLAG tag (8 amino acids) that can be captured via an M2 antibody. This system is advantageous as it provides identical selectivity toward all peptide barcodes, which can significantly improve recovery yield after the enrichment, and enable quantitative biochemical analysis with western blot during testing.

### Design of Transcription Units:

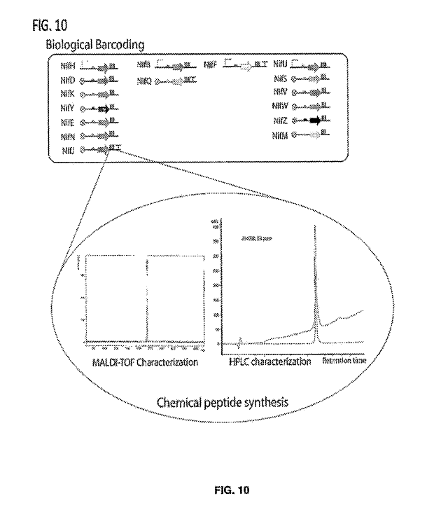

In a further refinement, transcription units were designed that harbor individually distinguishable peptide barcodes within each transcription unit. The transcription units, each including a promoter, ribosome binding site (RBS), nif gene and terminator were constructed with Type Ifs restriction sites at the end of each cluster so that full cluster can be constructed through a high-throughput pipelines. In addition, a set of sixteen peptides was chemically synthesized and tested for applicability to mass spec analysis via MALDI-TOF and HPLC (see FIG. 10). The resulting peptides are further purified to have purity higher than >75% and used as a gold standard for further mass spec and biochemical analysis.

### Amber Suppressor System:

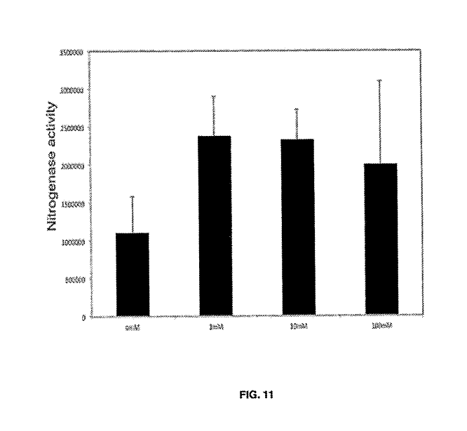

Trials were conducted to assess the expected toxicity upon expression of the amber suppressor system (see FIG. 11). Noting that addition of arabinose generally stimulates E. coli growth, the observed slight decrease in nitrogenase activity with increasing levels of amber suppression implies a minor toxic effect. With the aid of the epitope tagged peptide barcodes, optimal conditions for suppressor induction are investigated via immunoblot with the M2 antibody that targets the 1X flag tag present in the peptide barcode.

### Peptide Barcode Assessment:

For the trials each barcode contained TEV cleavage site, a differential mass tag and 1X FLAG peptide, connected to the one of the transcribed Nif proteins by amber stop codon. A transcription unit containing a promoter, an RBS, a CDS for NifF protein and a terminator was constructed as a prototype to test the expression of barcode tag and scalability of the system.

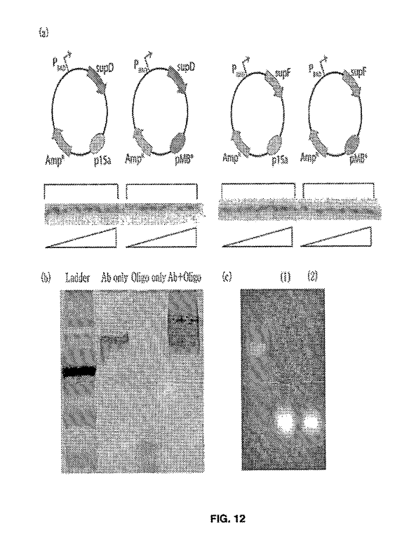

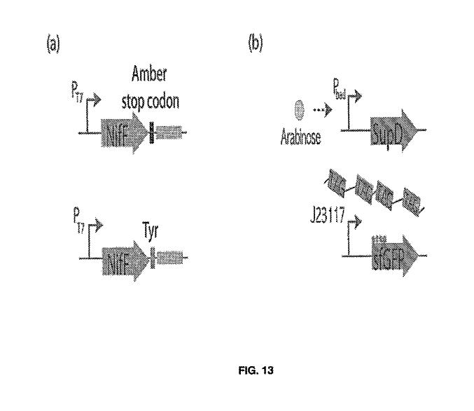

Under the presence of amber suppressor tRNA coding vector, western blotting confirmed the presence of FLAG tagged peptide barcodes (FIG. 12A, below). However, the uninduced negative controls also produced a band at the same molecular weight, indicating issues with the inducibility of the amber suppression system. Initially, after immunoprecipitation of FLAG tagged peptide and its TEV cleavage, we were not able to detect the designated peptide mass peak in MALDI experiment. It was hypothesized that this was due to either (i) lower sensitivity of the mass spectrometry than the western blotting or (ii) loss of peptide through IP steps or (iii) possible false positive issues in western blotting detection.

### Development of Nucleic Acid Based Detection

A PCR-based approach was developed to detect and quantitate protein expression level with higher sensitivity. This technique enables to measure protein levels by characterizing DNA (e.g. via next-generation sequencing), as opposed to LC/MS and allows new levels of sensitivity and multiplexing.

To develop this technique, an antibody was conjugated with HPLC-purified 70 bp oligos using sHynic and s4-FB modification/conjugation chemistry (FIG. 12B, 12C). Based on this approach, the signal could be amplified at 1/100 of the antibody concentration used previously.

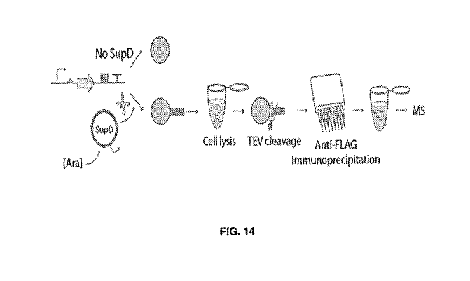

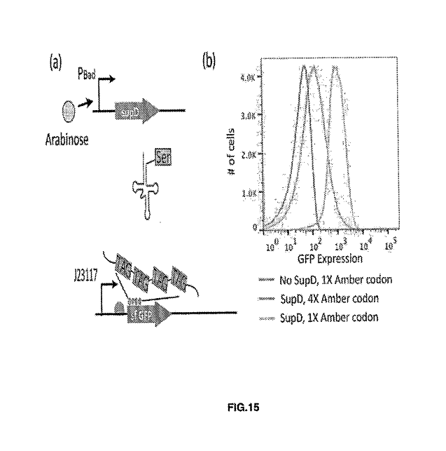

As a way to improve IP efficiency and inducibility of system, two debugging toolkits were developed. To confirm the expression of the system, a point mutation was generated at the amber stop codon, changing it to encode Tyr (FIG. 13A). In addition, a fluorescence protein based system was designed to test the inducibility of the suppressor system. This system contains four Ser amino acids mutated to the amber stop codon. With this scheme, if the suppressor system (SupD) is functional, the read through of the stop codon would happen and SupD will incorporate Ser to GFP protein instead of halting translation (FIG. 13B). Based on the inducibility test system constructed we have inducibility was optimize to attain 20-fold dynamic range in GFP expression upon induction of suppressor tRNA system.

In addition the barcode system was introduced in the complex metabolic pathways for lycopene synthesis. The transcription unit, in which this barcodes are encoded, also has ribozyme insulator, sRNA barcode and a double terminator to enable precise and flexible control of the expression.

### Example 6—Inducible Expression of Peptide Barcodes

Inducibility in expression of polypeptide identifier elements is a crucial design parameter required for protecting protein of interest from its chemical interactions with barcode elements (FIG. 17). Peptide barcodes with epitope tags for antibody binding often contain amino acids with charged residues as a part of the epitope tag and can easily interact with protein of interest thereby altering the protein's properties. By using amber suppressor tRNA (SupD), controlled expression of peptide barcodes achieved and is contingent on the inducible expression of suppressor tRNA.

In this example system, the amber suppressor tRNA (SupD), is expressed from pBAD promoter under the presence of an arabinose inducer, and is charged with serine amino acid. This suppressor tRNA replaces the end factor that used to be located in ribosomes upon reading the amber stop codon and instead inserts a serine amino acid to the polypeptide to allow protein synthesis to continue and thereby incorporate the peptide barcode encoded behind the stop codon.

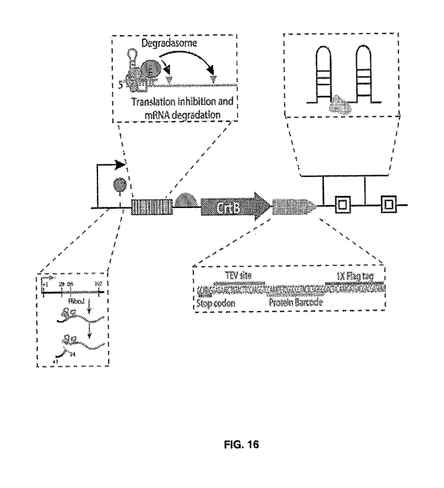

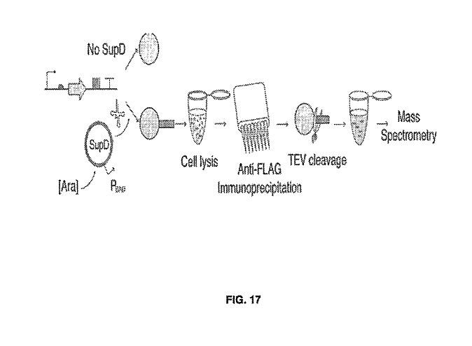

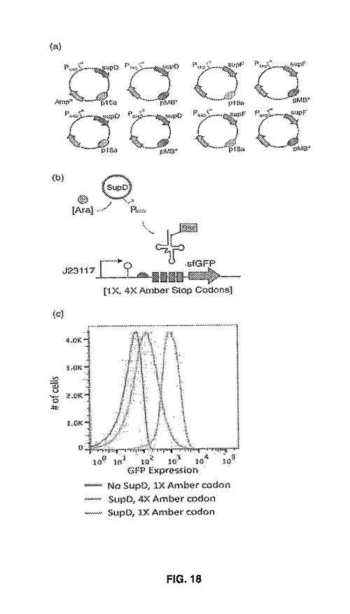

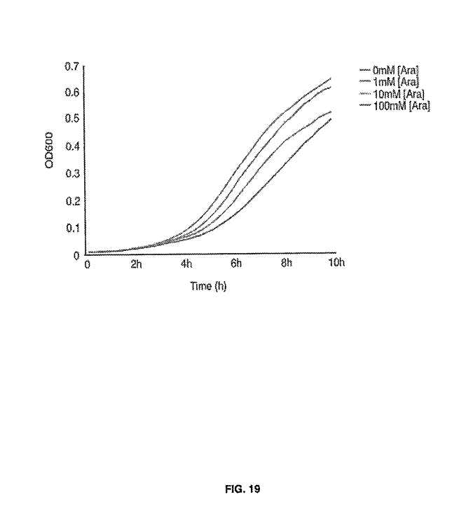

In order to develop an inducible amber stop codon suppression system that provides polypeptide identifier element synthesis control, an assay system that can confirm suppression of amber stop codon with fluorescence protein expression was constructed. A system that has one or four amber stop codons located relative to an N-terminus of sfGFP was constructed. See FIG. 18(b). Without the presence of amber suppressor tRNA, sfGFP synthesis is terminated at one or more of the up to four amber stop codons located and no fluorescence protein is expressed. The amber suppressor tRNA (SupD) was encoded in a separate plasmid. Once the amber suppressor tRNA (SupD) is induced and expressed, the amber stop codon on N-terminus of the sfGFP is suppressed and protein synthesis continues to generate full sfGFP proteins with fluorescence. This system has been used to implement a simple logic circuit based on amber suppressor tRNA (SupD), and adapted to test and optimize design parameters such as promoter, plasmid copy number, and different types of amber suppressor tRNAs (SupF and SupD). See FIG. 18(a). With this system, an improved amber suppressor tRNA expression system with an up to 10-fold increase in fluorescence expression after the expression of amber suppressor tRNA was achieved. See FIG. 18(c).

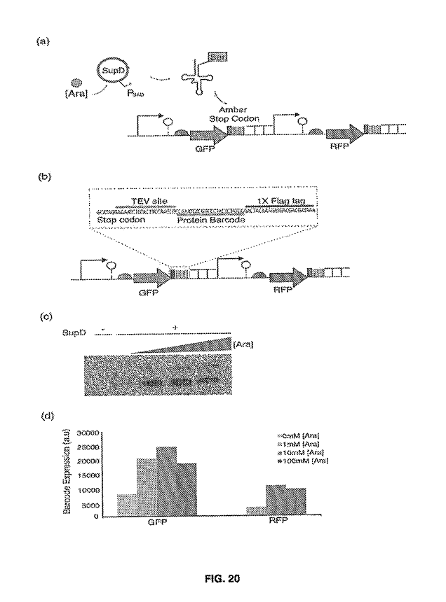

While expression of amber suppressor tRNA could impart global alterations in protein expression and function by changing translational reading frame, the system showed minor toxicity as determining a growth assay. See FIG. 19. After optimizing the SupD expression system, a monocistronic system with two transcription units each of which contains a promoter, a ribozyme insulator, an RBS, a fluorescence protein followed by an amber stop codon and a peptide barcode followed by a double terminator was constructed. See FIG. 20(a). In this system, each fluorescent protein is represented with a specific polypeptide identifier element having a distinguishable mass (FIG. 20(b)) and these polypeptide identifier elements further contain a FLAG epitope tag allowing for enrichment of barcoded populations only (FIG. 17). With this two-plasmid-based system, quantified SupD suppression dependent expression of polypeptide identifier elements was demonstrated. See FIGS. 20(c) and (d). Once an optimal induction condition for these constructs was found, enrichment and purification protocols were optimized in order to reduce sample complexity of cell lysate, which can hinder detection and analysis of polypeptide identifier elements (FIG. 17).

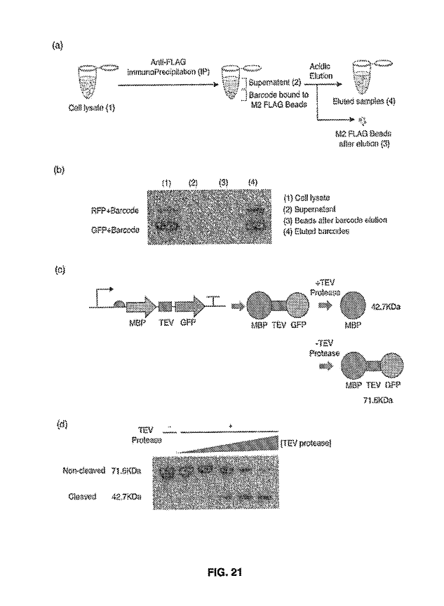

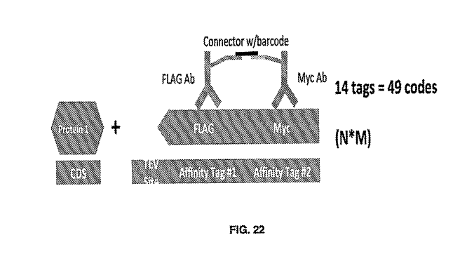

First, the cell lysates are immunoprecipitated using an anti-FLAG M2-bead with over >50% efficiency, confirmed by serial dilution to lower the concentration below the detection limit of western blotting. See FIG. 21(a). These enriched populations of proteins were then digested with highly stringent TEV protease. In most cases, thorough trypsin digestion is a key step in mass-spectrometry-based proteomics sample preparation in order to make each peptide small enough to fly through the detector. However, due to the low stringency of the trypsin recognition site, cellular proteome cleaved with trypsin dramatically increased the level of sample complexity, which impedes detection of target peptides from samples. By genetically encoding a TEV protease recognition site, these barcode constructs contain a TEV protease recognition site that can only be cleaved with TEV protease, thereby minimally increasing sample complexity. Therefore, we sought optimal condition for the TEV digestion using Maltose Binding Protein (MBP) and GFP conjugate protein spanned by TEV protease target site were determined. See FIG. 21(b). This MBP-GFP conjugate not only enables detection of protein expression by confirming fluorescence protein expression, but also allows for specific detection of the size of protein using anti-MBP antibody. TEV digestion of MBP-TEV-GFP fusion protein exhibited TEV concentration dependent increase of the protease activity. See FIG. 21(d). However, changing context of gene expression from MBP to two monocistronic fluorescence protein caused huge loss of TEV activity, necessitating further optimization of expression cassettes. Once this protein is expressed and is cleaved specifically with TEV protease, the released barcode can be purified through size exclusion column to further reduce the sample complexity by enriching small size of the peptide barcodes, and then analyzed with mass spectrometry.

### Example 7—Design of Proximity Ligation Probes

A initial set of constructs encoding a polypeptide identifier element comprising two affinity tags were designed as shown in the following table.

| 3 Prime | Target | Target | 5 Prime | Target | Target |
|---|---|---|---|---|---|
| Tag ID | 1 | Prot Seq | Tag ID | 2 | Prot Seq |
| 1 | Flag | DYKDDDDK | 1 | c-Myc | EQKLISEE |
| | | | | | DL |
| 2 | HA | YPYDVPDY | 2 | HIS | HHHHHH |
| | | A | | | |
| 3 | VSV-G | YTDIEMNR | 3 | V5 | GKPIPNPL |
| | | GLK | | | LGLDST |

A proximity ligation method was applied to detect the two proximate affinity tags. PCR primers were designed to detect a successful ligation product. A quantitative PCR analysis of PLA ligation was conducted. As shown in FIG. 24, the proximity ligation process produces a higher signal compared to free probes+connector in solution versus non probes (i.e. background qPCR of the primers. FIG. 25 shows the ability to make oligo antibody conjugated products, the probes for the proximity ligation assay and proximity extension assay the bands at 25 KDa are the light chain of the antibody, the 50 KDa band is the heavy chain, the 75 KDa band is the heavy chain+1×25 KDa oligo, the 100 kDa band is the heavy chain plus 2×25 KDa oligos. FIG. 26 show a test of proximity probes on positive control commercially available proteins. In this case GFP with a HIS tag. Anti-gfp mouse IgG conjugated to a 3′ free oligo and anti-His tag mouse Igg conjugated to a 5′ free oligo were used. The graph shows the signal of a real time qPCR reaction indicating the number of copies of the product of the PLA test. PLA was performed with an excess of probes of both types to the target molecule for about 5 fold and minimum and final ratio of 20 or more fold of connector to probes.

The following provides a general design protocol for designing additional polypeptide identifiers comprising two affinity tags for use in a PLA or PLE type approach. Oligonucleotides ordered from IDT as follows as double stranded DNA:

1.
5′atacatatgcaccaccaccaccaccacGTGGCGCGTGGCTATATGG
ATTATCGTGGTtaactcgagata3′
2.
5′atacatatgcaccaccaccaccaccacTTTGATTTTGAAGCGGGTA
AAgaagaataactcgagata3′
3.
5′atacatatgcaccaccaccaccaccacCTGGGCAGCCTGAGCGGCA
AAGAAtaactcgagata′
4.
5′atacatatgcaccaccaccaccaccacTTTAGCTTTTATGGCATGC
GTAGCtaactcgagata3′
5.
5′atacatatgcaccaccaccaccaccacGCGACCATGCAGAGCACCG
GCCACAGCCGTCAGtaactcgagata3′
6.
5′atacatatgcaccaccaccaccaccacTTTGGTTTTGATATGGCGG
GTAGCGATtaactcgagata3′
7.
5′atacatatgcaccaccaccaccaccacTTTAGCACCGGCATGGAAG
GCGATTATtaactcgagata3′
8.
5′atacatatgcaccaccaccaccaccacATGGGTCTGCGTGGCACC
CGTAAAAGCCAGtaactcgagata3′

To encode the following final amino acid sequences with varying polarity/charge and size properties:

1.
HHHHHHVARGYMDYRG
2.
HHHHHHFDFEAGKEE
3.
HHHHHHLGSLSGKE
4.
HHHHHHFSFYGMRS
5.
HHHHHHATMQSTGHSRQ
6.
HHHHHHFGFDMAGSD
7.
HHHHHHFSTGMEGDY
8.
HHHHHHMGLRGTRKSQ

The pET-24b(+) plasmid vector (Novagen) and above inserts were digested with NdeI restriction enzyme and XhoI restriction enzymes. The ligation was performed with T4 DNA ligase (New England Biolabs) and transformed into Escherichia coli DH5α. Colonies were grown on LB agar with 25 μg/mL Kanamycin. The verification of constructs was done by restriction digest analysis of plasmids with NheI and vectors not digested by the enzyme were selected for further analysis. The vectors were also verified by sequencing.

The verified plasmid vectors were transformed into E. coli BL21 (DE3) competent cells followed by selection on Luria Bertani (LB) agar with 25 μg/mL Kanamycin. Colonies were cultured in 10 mL volumes of double strength yeast extract (2×YT) media with 25 μg/mL Kanamycin and split into two 5 mL cultures after 3 hrs of growth with shaking at 37° C. One half was induced after 4 hrs of growth at 37° C. with a final concentration of 1 mM Isopropyl β-D-1-thiogalactopyranoside (IPTG) overnight. Both uninduced and induced overnight cultures were harvested and lysed by B-PER, partially purified and analyzed by MALDI/MS analysis.

### Proximity Ligation Approach

### The Following Tags Will be Generated for PLA):

### GFP-L-FLAG-L-HIS-L-Capture tag

### GFP-L-FLAG-L-MYC-L-Capture tag

### GFP-L-HA-L-MYC-L-Capture tag

### GFP-L-HA-L-HIS-L-Capture tag

(Capture tag encodes a tag technology available by Thermo Fisher)
L-Linker amino acid sequence is GGGGSGGGGS and is used widely in literature, amino acid sequences for epitope tag are standard and used widely in literature)

### Cloning of Green Fluorescent Protein (GFP)-Double Epitope Tagged Proteins:

GFP was Polymerase Chain Reaction (PCR) amplified from with forward primer 5′ atatgctagccgtaaaggagaagaactt3′ and reverse primer 5′ atatctcgagatactgcagtttgtatagttcatccatgc 3′ with NEBNext 2×PCR mastermix from a vector obtained from the Voigt lab at MIT. The PCR product of approximately 780 bps was verified by 1% agarose gel electrophoresis with E-Gel® (Invitrogen). The PCR product and pET-24b(+) plasmid vector (Novagen) was digested with NheI and XhoI restriction enzymes and ligated with T4 DNA polymerase (New England Biolabs) followed by transformation in Escherichia coli DH5α and selection on LB supplemented with 25 μg/mL Kanamycin. The constructs were verified by restriction digest analysis and one verified construct was named pET-24b(+) RDGFPHis and used for further analysis.

The epitope tags to be encoded in the sequence were designed as described below:

Nucleotide sequences were ordered as forward (F) and reverse (R) primers and complementary primers (F and R) duplexed for generation of double stranded inserts (All sequences below are listed 5′ to 3′):

GFP-L-FLAG-L-HIS-L-Capture_F
atactgcagggtggtggtggtagtggtggtggtggtagtGACTACAA
AGACGATGACGACAAGggtggtggtggtagtggtggtggtggtagtC
ACCACCACCACCACCACggtggtggtggtagtggtggtggtggtagt
gaaccggaagcgtaactcgagata
GFP-L-FLAG-L-MYC-L-Capture_F
atactgcagggtggtggtggtagtggtggtggtggtagtGACTACAA
AGACGATGACGACAAGggtggtggtggtagtggtggtggtggtagtG
AACAAAAACTCATCTCAGAAGAGGATCTGggtggtggtggtagtggt
ggtggtggtagtgaaccggaagcgtaactcgagata
GFP-L-HA-L-MYC-L-Capture_F
atactgcagggtggtggtggtagtggtggtggtggtagtTACCCATA
CGATGTTCCAGATTACGCTggtggtggtggtagtggtggtggtggta
gtGAACAAAAACTCATCTCAGAAGAGGATCTGggtggtggtggtagt
ggtggtggtggtagtgaaccggaagcgtaactcgagata
GFP-L-HA-L-HIS-L-Capture_F
atactgcagggtggtggtggtagtggtggtggtggtagtTACCCATA
CGATGTTCCAGATTACGCTggtggtggtggtagtggtggtggtggta
gtCACCACCACCACCACCACggtggtggtggtagtggtggtggtggt
agtgaaccggaagcgtaactcgagata
GFP-L-FLAG-L-HIS-L-Capture_R
tatctcgagttacgcttccggttcactaccaccaccaccactaccac
caccaccGTGGTGGTGGTGGTGGTGactaccaccaccaccactacca
ccaccaccCTTGTCGTCATCGTCTTTGTAGTCactaccaccaccacc
actaccaccaccaccctgcagtat
GFP-L-FLAG-L-MYC-L-Capture_R
tatctcgagttacgcttccggttcactaccaccaccaccactaccac
caccaccCAGATCCTCTTCTGAGATGAGTTTTTGTTCactaccacca
ccaccactaccaccaccaccCTTGTCGTCATCGTCTTTGTAGTCact
accaccaccaccactaccaccaccaccctgcagtat
GFP-L-HA-L-MYC-L-Capture_R
tatctcgagttacgcttccggttcactaccaccaccaccactaccac
caccaccCAGATCCTCTTCTGAGATGAGTTTTTGTTCactaccacca
ccaccactaccaccaccaccAGCGTAATCTGGAACATCGTATGGGTA
actaccaccaccaccactaccaccaccaccctgcagtat
GFP-L-HA-L-HIS-L-Capture_R
tatctcgagttacgcttccggttcactaccaccaccaccactaccac
caccaccGTGGTGGTGGTGGTGGTGactaccaccaccaccactacca
ccaccaccAGCGTAATCTGGAACATCGTATGGGTAactaccaccacc
accactaccaccaccaccctgcagtat

This vector and double stranded inserts listed above were digested with PstI and XhoI restriction enzymes and ligated. The constructs were transformed into DH5a and plated on LB supplemented with 25 μg/mL Kanamycin. The constructs were verified by restriction digest analysis and sequencing, and the one correct vector of each transformed into E. coli BL21(DE3) and selected on LB supplemented with 25 μg/mL Kanamycin.

The final vectors were named according to the epitope encoded:

pET-24b(+) RDGFPFLAGHIS encoding GFP-L-FLAG-L-HIS-L-Capture tag
pET-24b(+) RDGFPFLAGMYC encoding GFP-L-FLAG-L-MYC-L-Capture tag
pET-24b(+) RDGFPHAMYC encoding GFP-L-HA-L-MYC-L-Capture tag
pET-24b(+) RDGFPHAHIS encoding GFP-L-HA-L-HIS-L-Capture tag

Colonies were cultured in 10 mL volumes of double strength yeast extract (2×YT) media with 25 μg/mL Kanamycin and split into two 5 mL cultures after 3 hrs of growth with shaking at 37° C. One half was induced after 4 hrs of growth at 37° C. with a final concentration of 1 mM Isopropyl β-D-1-thiogalactopyranoside (IPTG) overnight. Both uninduced and induced overnight cultures were harvested and lysed by B-PER, partially purified and analyzed by MALDI/MS analysis, ELISA and PLA.

### Amino Acids Sequences of Tags are:

GFP-L-FLAG-L-HIS-L-Capture
tagMASRKGEELFTGVVPILVELDGDVNGHKFSVSGEGEGDATYGKL
TLKFICTTGKLPVPWPTLVTTFGYGVQCFARYPDHMKQHDFFKSAMP
EGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQ
QNTPIGDGPVLLPDNHYLSTQSALSKDPNEKRDHMVLLEFVTAAGIT
HGMDELYKLQGGGGSGGGGSDYKDDDDKGGGGSGGGGSHHHHHHGGG
GSGGGGSEPEA
GFP-L-FLAG-L-MYC-L-Capture
tagMASRKGEELFTGVVPILVELDGDVNGHKFSVSGEGEGDATYGKL
TLKFICTTGKLPVPWPTLVTTFGYGVQCFARYPDHMKQHDFFKSAMP
EGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQ
QNTPIGDGPVLLPDNHYLSTQSALSKDPNEKRDHMVLLEFVTAAGIT
HGMDELYKLQGGGGSGGGGSDYKDDDDKGGGGSGGGGSEQKLISEED
LGGGGSGGGGSEPEA
GFP-L-HA-L-MYC-L-Capture tag
MASRKGEELFTGVVPILVELDGDVNGHKFSVSGEGEGDATYGKLTLK
FICTTGKLPVPWPTLVTTFGYGVQCFARYPDHMKQHDFFKSAMPEGY
VQERTIFFKDDGNYKTRAEVKFEGDTLVNRIELKGIDFKEDGNILGH
KLEYNYNSHNVYIMADKQKNGIKVNFKIRHNIEDGSVQLADHYQQNT
PIGDGPVLLPDNHYLSTQSALSKDPNEKRDHMVLLEFVTAAGITHGM
DELYKLQGGGGSGGGGSYPYDVPDYAGGGGSGGGGSEQKLISEEDLG
GGGSGGGGSEPEA
GFP-L-HA-L-HIS-L-Capture tag
MASRKGEELFTGVVPILVELDGDVNGHKFSVSGEGEGDATYGKLTLK
FICTTGKLPVPWPTLVTTFGYGVQCFARYPDHMKQHDFFKSAMPEGY
VQERTIFFKDDGNYKTRAEVKFEGDTLVNRIELKGIDFKEDGNILGH
KLEYNYNSHNVYIMADKQKNGIKVNFKIRHNIEDGSVQLADHYQQNT
PIGDGPVLLPDNHYLSTQSALSKDPNEKRDHMVLLEFVTAAGITHGM
DELYKLQGGGGSGGGGSYPYDVPDYAGGGGSGGGGSHHHHHHGGGGS
GGGGSEPEA

All publications, patents, and patent applications mentioned herein are incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

We claim:

1. A method of multiplex analysis of polypeptides in samples, comprising:
providing a sample comprising one or more cells or an acellular system;
segregating each sample, or a portion of each sample, into an individual discrete volume;
labeling one or more target polypeptides, one or more target nucleic acids, or both, that are expressed in each sample with an origin-specific nucleic acid identifier such that each labeled target polypeptide and/or target nucleic acid is labeled with the same or matching origin-specific nucleic acid identifier thereby indicating expression from the same sample;
generating a cDNA copy of the labeled one or more expressed target nucleic acids such that the origin-specific nucleic acid sequence is incorporated into the cDNA copy and detecting the sequence of the cDNA copy;
separating each labeled target polypeptide complex by type to generate target polypeptide specific fractions;
for each polypeptide specific fraction, removing the origin-specific nucleic acid identifiers and detecting the sequence of the origin-specific nucleic acid identifiers; and
grouping the expressed target polypeptides and target nucleic acids by common origin-specific nucleic acid identifiers, thereby determining the individual discrete volumes in which the target polypeptides and target nucleic acids were co-expressed.

2. The method of claim 1, further comprising amplifying the cDNA copy prior to detecting the sequence of the cDNA copy.

3. The method of claim 1, wherein the one or more cells or the acellular system are modified to comprise a nucleic acid construct or set of nucleic acid constructs encoding the one or more target polypeptides and/or target nucleic acids.

4. The method of any one of claims 1 to 3, wherein labeling the one or more target polypeptides comprises introducing into each individual discrete volume the origin specific nucleic identifier and reagents sufficient to conjugate the origin specific nucleic acid identifier directly to the one or more target polypeptides expressed in a given individual discrete volume.

5. The method of claim 4, wherein conjugation of the origin-specific nucleic acid identifier is achieved by conjugation with a cysteine side chain of the one or more target polypeptides, or a side chain of one or more unnatural amino acids.

6. The method of claim 5, wherein the cysteine is a C-terminal cysteine.

7. The method of any one of claims 1 to 3, wherein labeling the one or more target polypeptides and/or one or more target nucleic acids comprises introducing into each individual discrete volume a labeling substrate comprising one or more protein capture molecules comprising a same origin-specific nucleic acid identifier and/or one or more nucleic acid capture molecules (oligos) comprising the same origin-specific nucleic acid identifier, wherein the one or more protein capture molecules and/or one or more nucleic acid capture molecules bind to a corresponding target polypeptide or target nucleic acid.

8. The method of claim 7, wherein the individual discrete volume is a single droplet generated on a microfluidic device, and wherein labeling comprises merging each single droplet with a second single droplet, the second single droplet comprising a single copy of the labeling substrate, the protein capture molecules and/or nucleic acid capture molecules on the labeling substrate having the same origin-specific nucleic acid identifier.

9. The method of claim 8, wherein the labeling substrate is a hydrogel bead from which the protein capture molecules and nucleic acid capture molecules can be released.

10. The method of any one of claims 1 to 3, wherein separating each labeled target polypeptide complex into target polypeptide specific fractions comprises pooling all individual discrete volumes into a single pooled sample comprising all labeled target polypeptides complexes and separating each labeled target polypeptide complex based on a unique size of the labeled target polypeptide complex and thereby identifying each target polypeptide.

11. The method of claim 10, wherein the labeled target polypeptide complexes are separated from the pooled sample using HPLC.

12. A method for multiplex analysis of polypeptides in samples, comprising:
providing a sample comprising one or more cells or an acellular system, wherein the one or more cells or acellular system comprise a construct or set of constructs, each set of constructs encoding one or more target polypeptides with a polypeptide identifier element such that the expressed target polypeptide includes the polypeptide identifier element, the polypeptide identifier element comprising a variable peptide sequence portion, the variable peptide sequence portion having at least one distinct physically separable property;
segregating each sample into a individual discrete volume under conditions sufficient to allow expression of the expressed target polypeptide;
labeling the polypeptide identifier of the expressed target polypeptides with an origin specific nucleic acid identifier to generate a labeled polypeptide identifier complex;
removing the labeled polypeptide identifier complex from each expressed target polypeptide and separating each labeled polypeptide identifier complex based on at least one physical property of the labeled polypeptide identifier complex to generate labeled polypeptide identifier specific fractions;
for each fraction, removing and detecting the sequence of the origin-specific nucleic acid identifiers in that fraction; and
grouping the target polypeptides by common origin-specific nucleic acid identifiers, thereby identifying the individual discrete volumes in which the target polypeptides were expressed.

13. The method of claim 12, wherein labeling the polypeptide identifier of the expressed target polypeptide comprises introducing into each individual discrete volume the origin specific nucleic acid identifier and reagents sufficient to conjugate the origin specific nucleic acid identifier to the polypeptide identifier portion of each expressed target polypeptide in the individual discrete volume.

14. The method of claim 13, wherein conjugation of the origin-specific nucleic acid identifier is achieved by conjugation directly to the polypeptide identifier.

15. The method of claim 14, wherein the origin-specific nucleic acid identifier is conjugated to the polypeptide identifier via a cysteine side-chain.

16. The method of claim 15, wherein the cysteine is a C-terminal cysteine on the polypeptide identifier.

17. The method of claim 12, wherein the polypeptide identifier comprises one or more non-naturally occurring amino acids, and wherein conjugation of the origin-specific nucleic acid identifier is achieved by conjugation to one of the one or more non-naturally occurring amino acids using click chemistry.

18. The method of claim 12, wherein labeling the polypeptide identifier element comprises binding the polypeptide identifier element with a peptide binding molecule that recognizes an epitope within the polypeptide identifier element, and wherein the origin specific nucleic acid molecule is previously attached to the peptide binding molecule.

19. The method of claim 12, wherein each individual discrete volume is an individual well of a microwell plate.

20. The method of claim 12, wherein the individual discrete volume is a single droplet generated on a microfluidic device, and wherein labeling comprises merging each single droplet with a second single droplet, the second single droplet comprising the protein binding molecule labeled with the origin specific nucleic acid identifier or the origin specific nucleic identifier and reagents sufficient to conjugate the origin specific nucleic acid identifier directly to the polypeptide identifier, each individual discrete volume receiving the same origin-specific nucleic acid identifier.

21. The method of claim 20, wherein the protein binding molecules are releasably attached to a substrate within the second droplet.

22. The method of claim 21, wherein the substrate is a hydrogel bead.

23. The method of claim 18, wherein the protein binding molecule is an antibody binding an affinity tag on the polypeptide identifier.

24. The method of claim 12, wherein the variable peptide sequence portion of the polypeptide identifier element is physically separable based on one or more of amino acid sequence, sequence length, charge, size, molecular weight, or hydrophobicity.

25. The method of claim 24, wherein the variable length peptide portion is separable based on size.

26. The method of claim 25, wherein each variable length peptide portion has a unique sequence length of between 2 and 200 amino acids.

27. The method of claim 12, wherein separating the labeled polypeptide identifier complexes is done using mass-spectral analysis, chromatography, or a combination thereof.

28. The method of claim 12, wherein separating the each labeled peptide barcode complex into peptide barcode specific fractions comprises pooling all individual discrete volumes into a single pooled sample and separating each labeled target polypeptide complex based on a unique size of the labeled peptide barcode complex.

29. The method of claim 28, wherein the labeled peptide barcode complexes are separated from the pooled sample using HPLC.

30. A method of multiplex analysis of polypeptides in samples, comprising:
providing a sample comprising one or more cells or an acellular system, wherein the one or more cells or acellular system comprise a construct or set of constructs, each set of constructs encoding one or more coding sequences, each coding sequence encoding a target polypeptide and polypeptide identifier element such that the polypeptide identifier element is included in the expressed target polypeptide, the polypeptide identifier element comprising an affinity tag;
segregating each sample into an individual discrete volume under conditions sufficient to allow expression of the target polypeptides;
removing polypeptide identifier element from expressed target polypeptide and introducing a set of protein binding molecules that bind a corresponding affinity tag to form a labeled polypeptide identifier element complex, the protein binding molecules having an affinity tag-specific oligonucleotide identifier conjugated thereto and identifying the affinity tag bound by that protein binding molecule;
appending an origin-specific nucleic acid identifier to the affinity tag specific nucleic acid identifier to generate a labeled polypeptide identifier complex comprising a combined nucleic acid identifier;
removing and detecting the sequence of the combined nucleic acid identifier, whereby the affinity-tag portion identifies the expressed target polypeptide and the origin-specific portion identifies the individual discrete volume in which the target polypeptide was expressed; and
grouping the combined nucleic acid identifiers by common origin-specific identifier, thereby identifying the target polypeptides expressed in the same individual discrete volume.

31. The method of claim 30, wherein each individual discrete volume is an individual well of a microwell plate.

32. The method of claim 30, wherein each individual discrete volume is a single droplet generated on a microfluidic device, and wherein binding a protein binding molecule to the polypeptide identifier element comprises merging each single droplet with a second single droplet, the second droplet comprising a set of protein binding molecules and a separate origin-specific nucleic acid identifier unique to each droplet.

33. The method of claim 32, wherein the protein binding molecules and nucleic acid identifiers are releasably attached to a substrate within the second droplet.

34. The method of claim 33, wherein the substrate is a hydrogel bead.

35. The method of claim 32, wherein the merged droplet is then incubated under conditions sufficient to allow ligation of the origin specific nucleic acid identifier to the end of the affinity tag-specific nucleic acid identifiers of the protein binding molecule.

36. The method of claim 30, wherein the affinity tag is one of a FLAG tag, an E tag, a HA tag, a Myc tag, a GFP tag, a VS tag, an alkaline phosphatase tag, a RFP tag, a VSV-G tag, a T7 tag, an AU1 tag, an AU 5 tag, or a S tag.

37. The method of claim 30, further comprising enriching the labeled polypeptide identifier having a combined oligo nucleic identifier by removing unbound protein binding molecules prior to removing and detecting the sequence of the combined polypeptide identifier element.

38. The method of claim 37, wherein the polypeptide identifier element further comprises a universal enrichment epitope, wherein labeled polypeptide identifier elements are enriched by purification via the universal enrichment epitope.

39. A method of multiplex analysis of polypeptides in samples, comprising:
providing a set of samples, each sample comprising one or more cells or an acellular system, wherein each sample is segregated into an individual discrete volume and the one or more cells or acellular system in each sample comprise a nucleic acid construct or set of nucleic acid constructs, each nucleic acid construct comprising one or more coding sequences, each coding sequence encoding a target polypeptide with a polypeptide identifier element, the polypeptide identifier element comprising an affinity tag portion and a variable peptide sequence portion having at least one distinct physically separable property;
segregating each sample into an individual discrete volume under conditions sufficient to allow for expression of the target polypeptides;
labeling the polypeptide identifier element of the expressed target polypeptide by covalently coupling an origin-specific nucleic acid identifier to the variable peptide sequence portion of the polypeptide identifier to generate a labeled polypeptide identifier complex;
removing the labeled polypeptide identifier element complex from the expressed target polypeptide and generating affinity tag specific fractions by separating the labeled polypeptide identifier element complexes based on common affinity tags;
for each affinity tag specific fraction, further labeling each labeled polypeptide identifier element complex by adding an affinity-tag specific nucleic acid identifier to the existing origin-specific nucleic acid identifier;
separating each double labeled polypeptide identifier element complex based on at least the physically separable property of the variable peptide sequence portion of the polypeptide identifier to generate polypeptide identifier specific fractions;
for each polypeptide identifier specific fraction, labeling each doubling labeled polypeptide identifier element complex by appending a fraction-specific nucleic acid identifier to the existing origin-specific and affinity tag-specific nucleic acid identifiers to generate a combined nucleic acid identifier;
removing and detecting the sequence of the combined nucleic acid identifier, whereby the combined affinity tag specific and fraction specific nucleic acid identifiers identify the expressed target polypeptides and the origin specific nucleic acid identifier identifies the individual discrete volume in which each target polypeptide was expressed;
grouping the sequenced combined nucleic acid identifiers by common origin-specific nucleic acid identifier, thereby identifying target polypeptides that originated from the same individual discrete volume.

40. The method of claim 39, wherein each individual discrete volume is an individual well of a microwell plate.

41. The method of claim 39, wherein the origin-specific nucleic acid identifier is attached to a side chain of an amino acid of the variable peptide sequence portion.

42. The method of claim 39, wherein the side chain is a cysteine side chain.

43. The method of claim 42, wherein the cysteine is a C-terminal cysteine on the variable peptide sequence portion.

44. The method of claim 41, wherein the variable sequence portion comprises one or more non-naturally occurring amino acids, and wherein conjugation of the origin-specific nucleic acid identifier is achieved by conjugation to one of the one or more non-naturally occurring amino acid using a click chemistry reaction.

45. The method of any one of claim 39, 40, or 41, wherein the individual discrete volume is a single droplet generated on a microfluidic device, and wherein labeling the polypeptide identifier with an origin-specific nucleic acid identifier comprises merging each single droplet with a second single droplet, the second single droplet comprising the origin-specific nucleic acid identifier.

46. The method of claim 45, wherein the protein binding molecules and origin-specific nucleic identifiers are bound to a substrate within the second droplet.

47. The method of claim 46, wherein the substrate is a hydrogel bead.

48. The method of claim 39, wherein the affinity tag is one of a FLAG epitope tag, an E tag, a HA tag, a Myc tag, a GFP tag, a V5 tag, an alkaline phosphatase tag, a RFP tag, a VSV-G tag, a T7 tag, an AU1 tag, an AU 5 tag, or a S tag.

49. The method of claim 39, wherein the variable peptide sequence portion is physically separable based on one or more of amino acid sequence, sequence length, charge, size, molecular weight, or hydrophobicity.

50. The method of claim 49, wherein the variable peptide sequence portion is separable based on size.

51. The method of claim 50, wherein each variable peptide sequence portion has a sequence length of between 2 and 200 amino acids.

52. The method of claim 39, wherein separating the polypeptide identifier is done using mass spectral analysis, chromatography, or a combination there.

53. A method of multiplex analysis of polypeptides in samples, comprising:
providing a set of samples, each sample comprising one or more cells or an acellular system, wherein the one or more cells or acellular system in each sample comprise a nucleic acid construct or set of nucleic acid constructs, each nucleic acid construct encoding a target polypeptide with a polypeptide identifier element, the polypeptide identifier comprising a unique combination of two affinity tags;
segregating each sample, or a portion of each sample, into individual discrete volumes under conditions sufficient to allow for expression of the target polypeptides;
labeling the polypeptide identifiers by introduction of protein binding molecules specific for a corresponding affinity tag, each protein binding molecule comprising a probe, each probe comprising an affinity tag identifier such that binding of any two protein binding molecules to the corresponding affinity tags on the polypeptide identifier places each nucleic acid identifier of the probes proximate to one another;
introducing a connector oligonucleotide into each individual discrete volume, each connector oligonucleotide comprising an origin-specific identifier unique to each individual discrete volume and an affinity tag identifier binding region that hybridizes to one affinity tag identifier or a specific combination of affinity tag identifiers to facilitate an extension or ligation reaction whereby the origin-specific identifier and the affinity tag identifier sequences are incorporated into the extension or ligation product;
amplifying and detecting the sequence of the extension or ligation product, wherein the affinity tag identifier sequences identify each expressed target polypeptide and the origin specific nucleic acid identifier identifies the individual discrete volume in which each expressed target polypeptide was expressed; and
grouping the sequenced ligation products by common origin-specific identifier thereby identifying target polypeptides that originated from the same individual discrete volumes.

54. The method of claim 53, wherein the one or more constructs encodes the connector nucleic acid.

55. The method of claim 53 or claim 54, wherein the connector oligonucleotide binds to a universal sequence portion on two proximate probes and facilitates an extension ligation reaction between the ends of the probes.

56. The method of claim 53 or claim 54, wherein the connector oligonucleotide comprises an origin specific nucleic acid identifier portion and a complementarity portion, the connector nucleic acid binding to a universal sequence portion of two proximate probes and facilitates a reverse transcriptase extension of each probe such that the origin-specific nucleic acid identifier and corresponding complementarity regions of the connector oligonucleotide are incorporated into the end of each probe such that the complementarity regions added to each proximate probe then hybridize to form a double-stranded portion that comprises the origin specific nucleic acid identifier, wherein the double-stranded portion comprising the origin specific nucleic acid identifier is then amplified and detected.

57. The method of claim 53 or 54, wherein the probes on the protein binding molecules comprise a first binding region to which a connector oligonucleotide can bind, an extension affinity tag identifier portion identifying the protein binding molecule, and a second binding region that can bind to a proximate probe, wherein the connector oligonucleotide binds to the probe via the first binding region such that it is proximate to a free end of a neighboring probe and such that a gap between the connector oligonucleotide and the neighboring probe is bridged by the extension affinity tag identifier portion of the probe to which the connector oligonucleotide is bound such that a ligation extension reaction is facilitated by the extension affinity tag portion to produce an extension product comprising the origin specific nucleic acid sequence of the connector oligonucleotide and the affinity tag identifier portion of the probes.

58. The method of claim 53 or claim 54, wherein the connector oligonucleotide binds to a probe through a first binding region and further comprises an origin specific nucleic acid sequence and a complementarity sequence, wherein the connector oligonucleotide serves as a template in a reverse transcription reaction that extends the free end of the probes to incorporate the origin specific nucleic acid sequence and the complementarity region into the extended end of the probes, wherein two such extended probes may then hybridize through the complementarity region to generated a hybridization product comprising the origin specific nucleic acid identifier.

59. The method of claim 53, wherein polypeptide identifier further comprises an enrichment tag.

60. The method of claim 59, wherein the labeled polypeptide identifiers are purified via the enrichment tag prior to generating the ligation or extension product or prior to amplifying and detecting the sequence of the ligation product.

61. The method of claim 53, wherein the polypeptide identifier further comprises a spacer between the two affinity tags.

62. The methods of any one of claims 51 to 57, wherein the probes on each protein binding molecule further comprise a forward primer site, a reverse primer site, or both.

63. The method of claim 53, wherein the two affinity tags are selected from the group consisting of a FLAG epitope tag, an E tag, a HA tag, a Myc tag, a GFP tag, a V5 tag, an alkaline phosphatase tag, a RFP tag, a VSV-G tag, a T7 tag, an AU1 tag, an AU 5 tag, and a S tag.

64. The method of claim 53, wherein the individual discrete volume is an individual well of a microwell plate.

65. The method of claim 53, wherein the individual discrete volume is a single droplet generated on a microfluidic device, and wherein labeling comprises merging each single droplet with a second single droplet, the second single droplet comprising the protein binding molecules and the connector oligonucleotide.

66. The method of claim 65, wherein the protein binding molecules and the connector oligonucleotide are attached to a substrate with the second droplet.

67. The method of claim 66, wherein the substrate is a hydrogel bead.

68. The method of claim 53, wherein the ligation product is amplified using helicase-dependent amplification.

69. The method of any one of claims 3, 12, 30, 39, and 53 wherein each construct encodes an amber stop codon between the target polypeptide and the peptide identifier.

70. The method of claim 69, wherein the one or more cells or acellular system comprises an inducible amber suppressor system, wherein the polypeptide identifier is included in the expressed target polypeptide only if the amber suppressor system is induced.

71. The method of claim 70, wherein the inducible amber suppressor system comprises a construct encoding amber suppressor tRNA that is expressed in the presence of an inducer.

72. The method of claim 70, wherein the amber suppressor is a SupF or SupD.

73. The method of any one of claims 70 to 72, wherein the amber suppressor system is inducible in the presence of arabinose.

74. The method of any one of claims 3, 12, 30, 39, and 53, wherein the constructs further encode a protease cleavage site between the target polypeptide and the polypeptide identifier.

75. The method of any one of claims 1, 12, 30, 39, and 53 wherein detecting the sequence of the origin-specific and/or combination nucleic acid identifiers comprises nucleic acid sequencing, amplification, hybridization, or any combination thereof.

76. The method of claim 53, wherein detecting the sequence of the origin-specific and/or combination nucleic acid identifiers comprises sequencing the origin-specific and/or combination nucleic acid identifiers using a next generation sequencing technique.

77. The method of claim 76, wherein the origin-specific nucleic acid identifier comprises a sequencing adaptor and/or universal priming site.

78. The method of claim 76, wherein the origin-specific nucleic acid identifiers further comprises a capture moiety, covalently or non-covalently linked.

79. The method of claim 78, wherein the capture moiety is biotin-16-UTP.

80. The method of any one of claims 1, 12, 30, 39, and 53, further comprising determining a count of the origin-specific nucleic acid identifiers, wherein the count indicates relative expression levels of the corresponding target polypeptide or target nucleic acid in the individual discrete volume.

81. The method of any one of claims 3, 12, 30, 39, and 53, wherein the constructs comprise synthetic genetic constructs comprising one or more cistrons, each cistron comprising a combination of regulatory elements and target polypeptide coding sequences.

82. The method of any one of claims 1, 12, 30, 39, and 53, wherein the origin specific nucleic identifiers, affinity specific nucleic acid identifiers, fraction specific nucleic acid identifiers, connector oligonucleotides further comprise a unique molecular identifier (UMI).

83. The method of claim 81, wherein the regulatory elements comprise promoters, ribozyme binding sites, terminators, RNAse cleavage sites, insulators, and spacers.

84. The method of claim 81, wherein the constructs are polycistronic.

85. The method of claim 81, wherein the one or more constructs encode a biosynthetic pathway.


SEQUENCE LISTING (42 sequences)

SEQ ID NO: 1 - protein, 8 aa, Artificial Sequence
  Synthetic polypeptide
DYKDDDDK

SEQ ID NO: 2 - protein, 9 aa, Artificial Sequence
  Synthetic polypeptide
YPYDVPDYA

SEQ ID NO: 3 - protein, 11 aa, Artificial Sequence
  Synthetic polypeptide
YTDIEMNRGLK

SEQ ID NO: 4 - protein, 10 aa, Artificial Sequence
  Synthetic polypeptide
EQKLISEEDL

SEQ ID NO: 5 - protein, 6 aa, Artificial Sequence
  Synthetic polypeptide
HHHHHH

SEQ ID NO: 6 - protein, 14 aa, Artificial Sequence
  Synthetic polypeptide
GKPIPNPLLGLDST

SEQ ID NO: 7 - DNA, 69 bp, Artificial Sequence
  Synthetic oligonucleotide
atacatatgcaccaccaccaccaccacgtggcgcgtggctatatggattatcgtggttaactcgagata

SEQ ID NO: 8 - DNA, 66 bp, Artificial Sequence
  Synthetic oligonucleotide
atacatatgcaccaccaccaccaccactttgattttgaagcgggtaaagaagaataactcgagata

SEQ ID NO: 9 - DNA, 63 bp, Artificial Sequence
  Synethic oligonucleotide
atacatatgcaccaccaccaccaccacctgggcagcctgagcggcaaagaataactcgagata

SEQ ID NO: 10 - DNA, 63 bp, Artificial Sequence
  Synthetic oligonucleotide
atacatatgcaccaccaccaccaccactttagcttttatggcatgcgtagctaactcgagata

SEQ ID NO: 11 - DNA, 72 bp, Artificial Sequence
  Synthetic oligonucleotide
atacatatgcaccaccaccaccaccacgcgaccatgcagagcaccggccacagccgtcagtaactcgagata

SEQ ID NO: 12 - DNA, 66 bp, Artificial Sequence
  Synthetic oligonucleotide
atacatatgcaccaccaccaccaccactttggttttgatatggcgggtagcgattaactcgagata

SEQ ID NO: 13 - DNA, 66 bp, Artificial Sequence
  Synthetic oligonucleotide
atacatatgcaccaccaccaccaccactttagcaccggcatggaaggcgattattaactcgagata

SEQ ID NO: 14 - DNA, 69 bp, Artificial Sequence
  Synthetic oligonucleotide
atacatatgcaccaccaccaccaccacatgggtctgcgtggcacccgtaaaagccagtaactcgagata

SEQ ID NO: 15 - protein, 16 aa, Artificial Sequence
  Synthetic sequence
HHHHHHVARGYMDYRG

SEQ ID NO: 16 - protein, 15 aa, Artificial Sequence
  Synthetic
HHHHHHFDFEAGKEE

SEQ ID NO: 17 - protein, 14 aa, Artificial Sequence
  Synthetic
HHHHHHLGSLSGKE

SEQ ID NO: 18 - protein, 14 aa, Artificial Sequence
  Synthetic
HHHHHHFSFYGMRS

SEQ ID NO: 19 - protein, 17 aa, Artificial Sequence
  Synthetic
HHHHHHATMQSTGHSRQ

SEQ ID NO: 20 - protein, 15 aa, Artificial Sequence
  Synthetic
HHHHHHFGFDMAGSD

SEQ ID NO: 21 - protein, 15 aa, Artificial Sequence
  Synthetic
HHHHHHFSTGMEGDY

SEQ ID NO: 22 - protein, 16 aa, Artificial Sequence
  Synthetic
HHHHHHMGLRGTRKSQ

SEQ ID NO: 23 - protein, 10 aa, Artificial Sequence
  Synthetic
GGGGSGGGGS

SEQ ID NO: 24 - DNA, 28 bp, Artificial Sequence
  Synthetic primer
atatgctagccgtaaaggagaagaactt

SEQ ID NO: 25 - DNA, 39 bp, Artificial Sequence
  Synthetic primer
atatctcgagatactgcagtttgtatagttcatccatgc

SEQ ID NO: 26 - protein, 6 aa, Artificial Sequence
  Synthetic construct
RDGFPH

SEQ ID NO: 27 - DNA, 165 bp, Artificial Sequence
  Synthetic primer
atactgcagggtggtggtggtagtggtggtggtggtagtgactacaaagacgatgacgacaagggtggtggtggtagtggtggtggtggtagtcaccaccaccaccaccacggtggtggtggtagtggtggtggtggtagtgaaccggaagcgtaactcgagata

SEQ ID NO: 28 - DNA, 177 bp, Artificial Sequence
  Synthetic primer
atactgcagggtggtggtggtagtggtggtggtggtagtgactacaaagacgatgacgacaagggtggtggtggtagtggtggtggtggtagtgaacaaaaactcatctcagaagaggatctgggtggtggtggtagtggtggtggtggtagtgaaccggaagcgtaactcgagata

SEQ ID NO: 29 - DNA, 180 bp, Artificial Sequence
  Synthetic primer
atactgcagggtggtggtggtagtggtggtggtggtagttacccatacgatgttccagattacgctggtggtggtggtagtggtggtggtggtagtgaacaaaaactcatctcagaagaggatctgggtggtggtggtagtggtggtggtggtagtgaaccggaagcgtaactcgagata

SEQ ID NO: 30 - DNA, 168 bp, Artificial Sequence
  Synthetic primer
atactgcagggtggtggtggtagtggtggtggtggtagttacccatacgatgttccagattacgctggtggtggtggtagtggtggtggtggtagtcaccaccaccaccaccacggtggtggtggtagtggtggtggtggtagtgaaccggaagcgtaactcgagata

SEQ ID NO: 31 - DNA, 165 bp, Artificial Sequence
  Synthetic primer
tatctcgagttacgcttccggttcactaccaccaccaccactaccaccaccaccgtggtggtggtggtggtgactaccaccaccaccactaccaccaccacccttgtcgtcatcgtctttgtagtcactaccaccaccaccactaccaccaccaccctgcagtat

SEQ ID NO: 32 - DNA, 177 bp, Artificial Sequence
  Synthetic primer
tatctcgagttacgcttccggttcactaccaccaccaccactaccaccaccacccagatcctcttctgagatgagtttttgttcactaccaccaccaccactaccaccaccacccttgtcgtcatcgtctttgtagtcactaccaccaccaccactaccaccaccaccctgcagtat

SEQ ID NO: 33 - DNA, 180 bp, Artificial Sequence
  Synthetic primer
tatctcgagttacgcttccggttcactaccaccaccaccactaccaccaccacccagatcctcttctgagatgagtttttgttcactaccaccaccaccactaccaccaccaccagcgtaatctggaacatcgtatgggtaactaccaccaccaccactaccaccaccaccctgcagtat

SEQ ID NO: 34 - DNA, 168 bp, Artificial Sequence
  Synthetic primer
tatctcgagttacgcttccggttcactaccaccaccaccactaccaccaccaccgtggtggtggtggtggtgactaccaccaccaccactaccaccaccaccagcgtaatctggaacatcgtatgggtaactaccaccaccaccactaccaccaccaccctgcagtat

SEQ ID NO: 35 - protein, 12 aa, Artificial Sequence
  Synthetic vector
RDGFPFLAGHIS

SEQ ID NO: 36 - protein, 12 aa, Artificial Sequence
  Synthetic vector
RDGFPFLAGMYC

SEQ ID NO: 37 - protein, 10 aa, Artificial Sequence
  Synthetic vector
RDGFPHAMYC

SEQ ID NO: 38 - protein, 10 aa, Artificial Sequence
  Synthetic vector
RDGFPHAHIS

SEQ ID NO: 39 - protein, 291 aa, Artificial Sequence
  Synthetic tag
  misc_feature (1)..(1): Xaa can be any naturally occurring amino acid
XMASRKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKLQGGGGSGGGGSDYKDDDDKGGGGSGGGGSHHHHHHGGGGSGGGGSEPEA

SEQ ID NO: 40 - protein, 295 aa, Artificial Sequence
  Synthetic tag
  misc_feature (1)..(1): Xaa can be any naturally occurring amino acid
XMASRKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKLQGGGGSGGGGSDYKDDDDKGGGGSGGGGSEQKLISEEDLGGGGSGGGGSEPEA

SEQ ID NO: 41 - protein, 295 aa, Artificial Sequence
  Synthetic tag
MASRKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKLQGGGGSGGGGSYPYDVPDYAGGGGSGGGGSEQKLISEEDLGGGGSGGGGSEPEA

SEQ ID NO: 42 - protein, 291 aa, Artificial Sequence
  Synthetic tag
MASRKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKLQGGGGSGGGGSYPYDVPDYAGGGGSGGGGSHHHHHHGGGGSGGGGSEPEA